(19) United States
(12) Patent Application Publication
(10) Pub. No.: US 2020/0352918 A1
(43) Pub. Date: Nov. 12, 2020

(54) COMPOUNDS AND METHODS FOR TREATING OR PREVENTING FIBROSIS

(71) Applicants: Brigitte N. Gomperts, Los Angeles, CA (US); Preethi Vijayaraj, Los Angeles, CA (US)

(72) Inventors: Brigitte N. Gomperts, Los Angeles, CA (US); Preethi Vijayaraj, Los Angeles, CA (US)

(21) Appl. No.: 16/868,123

(22) Filed: May 6, 2020

Related U.S. Application Data
(60) Provisional application No. 62/844,541, filed on May 7, 2019.

(51) Int. Cl. A61K 31/4365; A61K 31/407; A61K 31/365; A61K 31/216; A61K 31/198; A61K 31/4412; A61K 31/4168; A61K 31/132; A61P 19/04

(52) U.S. Cl. CPC A61K 31/4365; A61K 31/407; A61K 31/365; A61K 31/216; A61P 19/04; A61K 31/4412; A61K 31/4168; A61K 31/132; A61K 31/198

(57) ABSTRACT

Disclosed herein are small molecules that inhibit fibrosis, and their use for treatment of fibrosis related diseases. Methods of identifying these small molecules using an in vitro fibrosis model are described.

### RELATED APPLICATION

This application claims the benefit of priority of U.S. Patent Application No. 62/844,541, filed May 7, 2019, which is hereby incorporated by reference herein in its entirety.

### GOVERNMENT SUPPORT

This invention was made with government support under Grant Numbers GM114259, TR000124 and TR001881, awarded by the National Institutes of Health. The government has certain rights in the invention.

### BACKGROUND

Progressive fibrosis across organs shares common cellular and molecular pathways involving chronic injury, inflammation and aberrant repair resulting in deposition of extracellular matrix, organ remodeling and ultimately organ failure. Fibrosis is characterized by over expression of transforming growth factorβ (TGFβ) family members and the abnormal and excessive buildup of extracellular matrix (ECM) components, such as fibrillar collagen. This accumulation of ECM triggers progressive organ remodeling and therefore organ dysfunction. Often this fibrotic process is driven by metabolic and inflammatory diseases that result in organ injury and perpetuate the fibrosis. The fact that many different diseases all result in the same fibrotic response in different organs such as the liver, kidney, lung and skin, speaks for a common disease pathogenesis.

Progressive organ fibrosis accounts for one third of all deaths worldwide, yet there are no known curative therapies. Currently, treatments are available for fibrotic disorders including general immunosuppressive drugs such as corticosteroids, and other anti-inflammatory treatments. However, the mechanisms involved in regulation of fibrosis appear to be distinctive from those of inflammation, and anti-inflammatory therapies are seldom effective in reducing or preventing fibrosis. Therefore, a need remains for developing treatments to reduce and prevent fibrosis and control fibrotic disorders.

### SUMMARY

Disclosed herein are methods of treating or preventing a fibrotic disease or disorder, including fibroproliferative disorders, comprising administering to a subject in need thereof one or more agents, such as fibrosis inhibitors.

Disclosed herein are methods of inhibiting fibrosis comprising administering to a subject in need thereof one or more agents as described herein. In some embodiments, the fibrosis comprises progressive fibrosis. In other embodiments, the fibrosis is fibroproliferative. In certain embodiments, the fibrosis is viral-infection-induced fibrosis, e.g., incident to COVID-19.

Also disclosed is an in vitro model fibrosis, including progressive fibrosis, and identification of fibrosis inhibitors with the model. Provided herein are agents for use in inhibiting fibrosis, including treatment of fibrotic diseases and disorders. Also provided herein are uses of agents for the manufacture of a medicament for inhibiting fibrosis, including treatment of fibrotic diseases and disorders.

In some embodiments of methods disclosed herein, the agent can be selected from

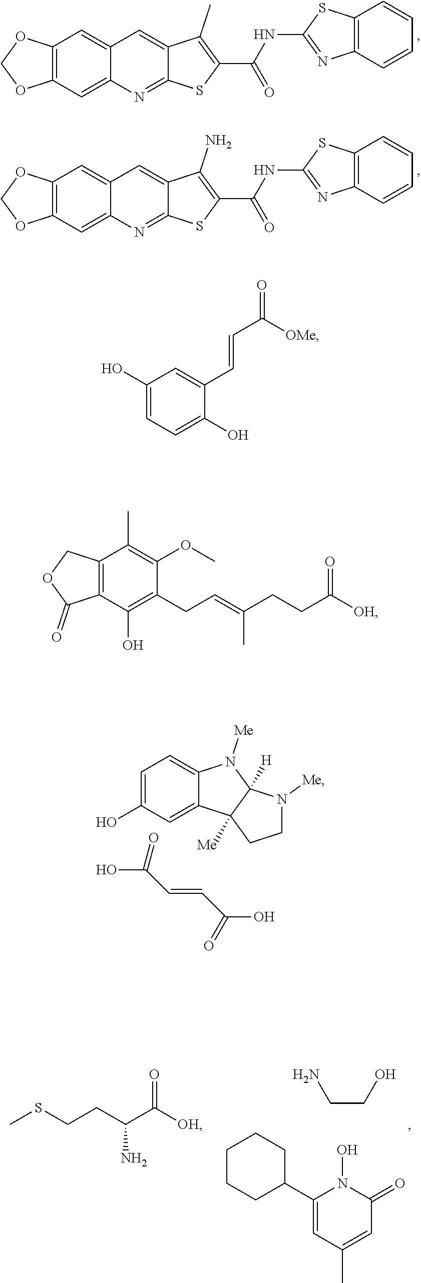

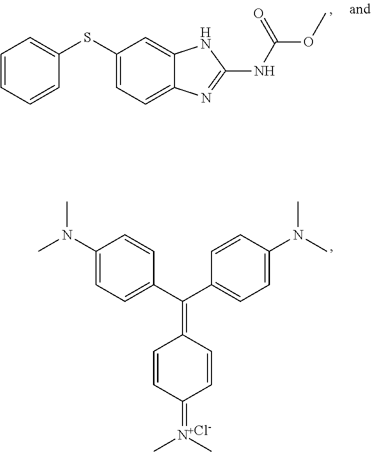

or a pharmaceutically acceptable salt thereof.

In certain preferred embodiments, the agent is

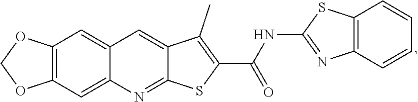 [SMILES: Cc1c(C(=O)Nc2nc3ccccc3s2)sc2nc3cc4c(cc3cc12)OCO4]

or a pharmaceutically acceptable salt thereof. In other preferred embodiments, the agent is

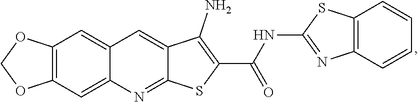 [SMILES: Nc1c(C(=O)Nc2nc3ccccc3s2)sc2nc3cc4c(cc3cc12)OCO4]

or a pharmaceutically acceptable salt thereof. In yet other preferred embodiments, the agent is

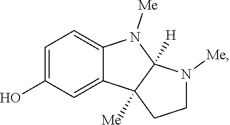 [SMILES: CN1CC[C@@]2(C)c3cc(O)ccc3N(C)[C@@H]12]

or a pharmaceutically acceptable salt thereof. In certain other preferred embodiments, the agent is

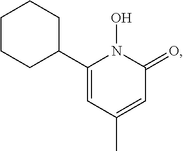

or a pharmaceutically acceptable salt thereof.

In some embodiments, the agent is present in a pharmaceutical composition comprising a pharmaceutically acceptable carrier.

Exemplary fibrotic diseases and disorders include, but are not limited to, interstitial lung disease, idiopathic pulmonary fibrosis, liver cirrhosis, liver fibrosis resulting from chronic hepatitis B or C infection, kidney disease, heart disease, and eye diseases including macular degeneration and retinal and vitreal retinopathy. Exemplary fibroproliferative disorders include, but are not limited to, systemic and local scleroderma, keloids and hypertrophic scars, atherosclerosis, and restenosis. Other fibrotic disorders include viral-infection-induced fibrosis, e.g., fibrosis associated with COVID-19.

Exemplary fibrotic diseases or disorders are selected from liver disease, kidney disease, idiopathic pulmonary fibrosis (IPF), heart failure, scleroderma, rheumatoid arthritis, Crohn's disease, ulcerative colitis, myelofibrosis, systemic lupus erythematosus, tumor invasion and metastasis, and chronic graft rejection. In some embodiments, the fibrotic disease or disorder is selected from systemic or local scleroderma, keloids, hypertrophic scars, atherosclerosis, restenosis, pulmonary inflammation and fibrosis, idiopathic pulmonary fibrosis, liver cirrhosis, fibrosis as a result of SARS-CoV-2 or chronic hepatitis B or C infection, kidney disease, heart disease resulting from scar tissue, macular degeneration, and retinal and vitreal retinopathy. In certain embodiments, methods disclosed herein inhibit excessive fibrosis formation occurring in the liver, kidney, lung, heart or pericardium, eye, skin, mouth, pancreas, gastrointestinal tract, brain, breast, bone marrow, bone, genitourinary, a tumor, or a wound.

### BRIEF DESCRIPTION OF THE FIGURES

FIG. 1A shows primary cultures of lung fibroblasts that expressed the fibroblast marker FSP1.

FIG. 1B shows primary fibroblasts were reprogrammed into iPSC and expressed pluripotency marker SOX2.

FIG. 1C shows the iPSCs were differentiated into cells that expressed markers that were largely mesenchymal-like cells (Vimentin, VIM).

FIG. 1D shows an overlaid histogram plot that depicts the relative SSEA4 fluorescence intensity (blue) of the mesenchymal-like cells compared to the unstained controls (grey).

FIG. 1E shows an overlaid histogram plot that depicts the relative CD326 (epithelial marker) fluorescence intensity (blue) of the mesenchymal-like cells compared to the unstained controls (grey).

FIG. 1F shows representative FACS plots revealing expression of monocyte/macrophage markers in the mesenchymal-like cells.

FIG. 1G shows a representative image of mesenchymal-like cell compared to a macrophage-like cell.

FIG. 1H shows characterization of primary fibroblasts (middle panel) and mesenchymal-like cells (right panel) using multi-color FACS.

FIG. 2A shows comparative IF images of primary fibroblasts and mesenchymal-like cell cultures stained for fibroblast marker (FSP-1) and mesenchymal marker (VIM).

FIG. 2B shows comparative IF images of primary fibroblasts and mesenchymal-like cell cultures.

FIG. 2C shows representative IF images of decellularized primary fibroblast (control) and iFA cultures stained for Collagen I.

FIG. 2D shows single positive cells for SSEA4, CD105 and CD326 (negative for CD45) that were sorted from the iFA model and re-cultured on 13 kPa hydrogels.

FIG. 2E shows the iFA model created from single positive cells of CD105, SSEA4 and CD326.

FIG. 3A shows phase-contrast images demonstrating propagation of mesenchymal-like cells on 13 kPa hydrogels.

FIG. 3B shows phase-contrast images demonstrating the development of the iFA phenotype only in cultures from mesenchymal-like cells.

FIG. 3C shows quantification of EdU positive cells in primary fibroblasts and mesenchymal-like cells grown on 13 kPa hydrogels related to FIG. 2B.

FIG. 3D shows fibrosis-related genes expression by qPCR in primary fibroblasts (control) and mesenchymal-like cells (iFA) cultured on 13 kPa hydrogels.

FIG. 3E shows a representative immunoblot analysis of the expression of Collagen I and α-SMA in primary fibroblasts (control) and mesenchymal-like cells (iFA).

FIG. 3F shows representative transmission electron microscopic (TEM) images showing ultrastructure of cells in the iFA model.

FIG. 4A shows active TGF-β secreted during the progression of the iFA phenotype.

FIG. 4B shows time-dependent levels of secreted TGF-β1 during the development of the iFA phenotype.

FIG. 5A shows PAI-1 promoter/luciferase construct-transfected mink lung epithelial cells that were incubated with various concentrations of human recombinant(r) TGF-β1.

FIG. 5B shows time-dependent levels of secreted TGF-β1 during the development of the iFA phenotype

FIG. 6A shows a representative image of the iFA model at D13 of culture, demonstrating senescent cells with SA-β-Gal staining.

FIG. 6B shows cytokine levels determined from conditioned media of the iFA model at D13 relative to D4.

FIG. 6C shows HMGB1 levels determined from conditioned media of the iFA model at D13 relative to D4.

FIG. 6D shows a representative image of the iFA model at day 13 stained for NF-κB p65.

FIG. 7 shows cytokine profiles of proteins secondary to acute-phase response in supernatants from the iFA model at D13 relative to D4 cytokines in AA5r versus DMSO, and AA5p versus DMSO-treated cells.

FIG. 8A shows relative secreted levels of HMGB1 in supernatants from the conditioned media of AA5-treated LSC cultures.

FIG. 8B shows a representative image of DMSO- and AA5-treated LSC stained for HMGB1.

FIG. 9 shows representative DMSO-treated, AA5-prevention (AA5p) and AA5-resolution (AA5r) iFA cultures immunostained for VIM and α-SMA (top panels) and Collagen I and α-SMA (bottom panels).

FIG. 10A shows a representative phase contrast image (top left) of the iFA model at D13 that was used to measure the elastic modulus. 3D Rendering of the AFM amplitude channel shows representative areas of 1, 2 and 3 from panel (top right and bottom panels).

FIG. 10B shows force versus distance curves measured on cells from FIG. 6A.

FIG. 11 shows quantification of stiffness of the cells in the DMSO-treated, AA5p and AA5r iFA model.

FIG. 12A shows a heat map summarizing fold change for 84 fibrosis-related genes exhibiting differential expression across the iFA model.

FIG. 12B shows densitometric analysis depicting fold change of α-SMA and Collagen I in fibrosis models.

FIG. 12C shows a heat map summarizing fold change of differential expression of cytokines and growth factors in supernatants of fibrosis models.

FIG. 12D shows HMGB1 levels determined from conditioned media in the fibrosis models.

FIG. 13A shows a representative immunoblot analysis of the expression of Collagen I and α-SMA in exogenously TGF-β-treated fibrosis models.

FIG. 13B shows representative IF images of Collagen I and α-SMA in exogenously TGF-β-treated fibrosis models.

FIG. 13C shows representative images of exogenously TGF-β-treated fibrosis models.

FIG. 14 shows a superimposed representative scatter plot showing the results from a single 96-well plate of the iFA-prevention assay.

FIG. 15 shows high content staining discrimination of cellular aggregates vs individual spindle-shaped live cells.

FIG. 16A shows prevention of the iFA phenotype in the well containing AA5 compared to DMSO control.

FIG. 16B shows the dose response of AA5 showing IC₅₀ of 0.9 μM.

FIG. 16C shows a pre-presentation of the assay performance with Z′ calculation demonstrating a robust assay performance using the number of cellular aggregates.

FIG. 16D shows the assay performance with Z′ calculation demonstrating the assay performance using the phenotypic cell index (PI).

FIG. 17A shows representative images from wells of the iFA model treated with 0.6 μM-10 μM AA5.

FIG. 17B shows comparative IF images of DMSO (top panel) and AA5-prevention (bottom panel) treated iFA model.

FIG. 17C shows EdU positive DAPI cells from FIG. 17B quantified for each time point.

FIG. 17D shows a representative immunoblot analysis of the expression of Collagen I and α-SMA in the DMSO and AA5-prevention samples.

FIG. 17E shows the time-dependent fold change in secreted POSTN and TIMP-3 in response to AA5-prevention treatment versus DMSO.

FIG. 18A shows secreted levels of HMGB1 in the iFA model during AA5p and AA5r treatments.

FIG. 18B shows ClueGO clustering analysis results of up- (red) and down-regulated (blue) genes in a pairwise comparison of AA5p treatment in a DMSO-treated iFA model.

FIG. 18C shows ClueGO clustering analysis results of up- (red) and down-regulated (blue) genes in a pairwise comparison of AA5r treatment in a DMSO-treated iFA model.

FIG. 18D shows a comparison of gene expression fold change levels of long-pentraxin family members PTX3 and NPTX1 and scavenger receptor CD163L1 in the AA5r and AA5p treated iFA model.

FIG. 18E shows the time-dependent fold increase in secreted PTX3 and NPTX1 on AA5p treatment.

FIG. 18F shows cytokine profiles of proteins secondary to acute-phase response in supernatants from the iFA model at D13 relative to D4 cytokines in AA5r versus DMSO, and AA5p versus DMSO-treated cells.

FIG. 19A shows the effect of AA5 on preventing fibrosis in the production of a shift in the gene expression levels in lung IPF tissue in contrast to healthy lung controls.

FIG. 19B shows the effect of AA5 on resolving fibrosis in the production of a shift in the gene expression levels in lung IPF tissue in contrast to healthy lung controls.

FIG. 19C shows a list of significantly enriched terms using ClueGO analysis of the AA5-mediated prevention iFA phenotype in the iFA model.

FIG. 19D shows a list of significantly enriched terms using ClueGO analysis of the AA5-mediated resolution iFA phenotype in the iFA model.

FIG. 20 shows the fold change of gene expression of ACTA2, COL1A2 and TGF-β3 in AA5-treated lung slice cultures (LSC) relative to DMSO-treated controls.

FIG. 21 shows the Rank Rank Hypergeometric Overlap (RRHO) analysis showing hypergeometric overlap between differentially expressed genes in the iFA model post AA5-prevention and post AA5-resolution treatments.

FIG. 22A shows schema illustrating the experiment to induce and treat ocular fibrosis in mice in a dose-dependent manner.

FIG. 22B shows the ocular surface inflammatory score in OVA-treated mice in fibrosis prevention studies.

FIG. 22C shows the ocular surface inflammatory score in OVA-treated mice in fibrosis resolution studies.

FIG. 22D shows the total collagen content in conjunctival tissue in naïve, OVA-, DMSO- and AA5-prevention treated animals following ocular scarring.

FIG. 22E shows the total collagen content in conjunctival tissue in naïve, OVA-, DMSO- and AA5-resolution treated animals following ocular scarring.

FIG. 22F shows representative Gomori Trichrome stained sections of whole mouse eyes.

FIG. 23A shows relative secreted levels of fibrosis-related proteins in supernatants of AA5-treated LSC cultures.

FIG. 23B shows representative FACS plots revealing expression of SSEA4+ population in LSC treated with DMSO or AA5.

FIG. 23C shows ClueGO clustering analysis results of up- (red) and down-regulated (blue) genes in a pairwise comparison of AA5- versus DMSO-treated LSCs.

FIG. 24 shows the list of significantly enriched terms in the ClueGO analysis from the pairwise comparison between AA5 and DMSO treated LSCs.

FIG. 25A shows the fold change of expression of PTX3, NPTX1 and CD163L1 in AA5-treated LSC relative to DMSO-treated controls 48 hours after treatment at mRNA levels.

FIG. 25B shows the fold change of expression of PTX3, NPTX1 and CD163L1 in AA5-treated LSC relative to DMSO-treated controls 48 hours after treatment at secreted protein levels.

FIG. 25C shows representative images from wells of the iFA model treated with DMSO, 10 μM AA5 or 3.75 μgm/ml IFNγ.

FIG. 25D shows the relative fold change of secreted proteins in LSCs treated with 3.75 μg/ml rIFNγ for 48 hours in comparison to DMSO-treated controls.

FIG. 26A shows a representative image of DMSO- and AA5-treated lung slice cultures (LSCs) stained for the proliferation marker PCNA.

FIG. 26B shows relative secreted levels of TGF-β proteins in supernatants of AA5-treated LSCs compared to DMSO-treated controls.

FIG. 26C shows gene expression analysis showing relative expression levels of fibrosis-related genes in LSCs treated with AA5 compared with DMSO.

FIG. 26D upper panel shows representative images of DMSO- and AA5 (10 μM)-treated LSCs stained for NPTX1. The lower panel shows representative images of DMSO- and AA5 (10 μM)-treated LSCs stained for scavenger receptor protein CD163L1 by immunohistochemistry.

FIG. 26E shows secreted levels of NPTX1 in the LSCs treated for 48 hours with DMSO and AA5.

FIG. 27A shows a representative still frames from a time-lapse series showing an invasive and progressive phenotype in the iFA model.

FIG. 27B shows quantitative data presented as mean±s.e.m depicting the expression of Collagen I and α-SMA in the iFA model and AA5-presolution cultures.

FIG. 27C shows representative IF staining for HMGB1 in DMSO-treated, AA5-prevention- and AA5-resolution-treated iFA model.

FIG. 27D shows an overlaid histogram plot that depict the relative SSEA4 fluorescence intensity in the iFA model with (red) and without (blue) AA5 treatment.

FIG. 28A shows unstained samples were gated using forward and side scatter (FSC-A and SSC-A) followed by SSC-W/SSC-H.

FIG. 28B shows unstained samples were gated using forward and side scatter (FSC-A and SSC-A) followed by FSC-W/FSC-H.

FIG. 28C shows unstained samples were gated using forward and side scatter (FSC-A and SSC-A) to select single cells.

FIG. 28D shows negative cells were then gated using the unstained controls.

FIG. 28E shows positive gating drawn using single stained controls.

FIG. 29A shows enrichment analysis of canonical pathways (1,188 MSigDB canonical pathways) in transcriptome data of RNA sequencing in the iFA model.

FIG. 29B shows a heatmap showing expression of genes (red/blue are up/down-regulated), encoding for pathways in core matrisome.

FIG. 29C shows a heatmap showing expression of genes (red/blue are up/down-regulated), encoding for pathways in the indicated factors.

FIG. 29D shows a heatmap showing expression of genes (red/blue are up/down-regulated), encoding for pathways in integrin 1.

FIG. 29E shows a heatmap showing expression of genes (red/blue are up/down-regulated), encoding for pathways in cytokine-cytokine receptor interaction.

FIG. 30A shows schema illustrating the experiment to induce and treat IPF in mice.

FIG. 30B shows hydroxyproline content in lung tissue collected on day 21 post bleomycin injury with and without AA5.

FIG. 30C shows representative H&E section of bleomycin treated lungs.

FIG. 30D shows the percent fibrotic area that was calculated for each lung lobe.

FIG. 30E shows H&E stained sections of bleomycin-treated lungs with and without AA5.

FIG. 30F shows representative IF images of naïve, vehicle and AA5 treated mice lungs.

### DETAILED DESCRIPTION

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In some embodiments, chemical structures are disclosed with a corresponding chemical name. In case of conflict, the chemical structure controls the meaning, rather than the name.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

In certain embodiments, compounds of the invention may be racemic. In certain embodiments, compounds of the invention may be enriched in one enantiomer. For example, a compound of the invention may have greater than about 30% ee, about 40% ee, about 50% ee, about 60% ee, about 70% ee, about 80% ee, about 90% ee, or even about 95% or greater ee. In certain embodiments, compounds of the invention may have more than one stereocenter. In certain such embodiments, compounds of the invention may be enriched in one or more diastereomer. For example, a compound of the invention may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., of Formula (I)). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of Formula (I)). A diastereomerically enriched mixture may comprise, for example, at least about 60 mol percent of one diastereomer, or more preferably at least about 75, about 90, about 95, or even about 99 mol percent.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, an agent that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the agents of the present invention. A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present invention. In certain embodiments, some or all of the disclosed agents in a formulation represented above can be replaced with the corresponding suitable prodrug, e.g., wherein a hydroxyl in the parent compound is presented as an ester or a carbonate or carboxylic acid.

An “effective amount”, as used herein, refers to an amount that is sufficient to achieve a desired biological effect. A “therapeutically effective amount”, as used herein, refers to an amount that is sufficient to achieve a desired therapeutic effect. For example, a therapeutically effective amount can refer to an amount that is sufficient to improve at least one sign or symptom of a fibrotic disease or disorder.

### Methods of Use

Fibrosis can be defined by the excessive accumulation of fibrous connective tissue (components of the extracellular matrix (ECM) such as collagen and fibronectin) in and around inflamed or damaged tissue, which can lead to permanent scarring, organ malfunction and, ultimately, death, as seen in end-stage liver disease, kidney disease, idiopathic pulmonary fibrosis (IPF) and heart failure. Fibrosis is a pathological feature of most chronic inflammatory diseases. Fibrosis is also a major pathological feature of many chronic autoimmune diseases, including scleroderma, rheumatoid arthritis, Crohn's disease, ulcerative colitis, myelofibrosis, systemic lupus erythematosus and Dupuytren's contracture. Fibrosis also influences tumor invasion and metastasis, chronic graft rejection and the pathogenesis of many progressive myopathies.

Cell-intrinsic changes in important structural cells can perpetuate the fibrotic response by regulating the differentiation, recruitment, proliferation and activation of extracellular matrix-producing myofibroblasts. In addition to abnormal and excessive buildup of extracellular matrix components, fibrosis is characterized by over-expression of transforming growth factorβ (TGFβ) family members which stimulates ECM synthesis by local fibroblasts. Fibrosis can be triggered by acute inflammatory reactions and aberrant wound-healing mechanisms. Macrophages that appear early in the wound-healing response are also major producers of TGF-β, which is one of the key drivers of fibrosis. TGF-β production correlates with the progression of liver, lung, kidney, skin and cardiac fibrosis. While monocytes, macrophages and neutrophils have important roles in the progression and resolution of fibrosis, other myeloid-lineage cells (such as mast cells, eosinophils and basophils) have also been implicated in the pathogenesis of fibrosis in multiple organ systems.

Given the common etiology of fibrosis, many fibrotic diseases and disorders are amenable to treatment or prevention with the agents disclosed herein. Exemplary fibrotic diseases and disorders include, but are not limited to, collagen disease, interstitial lung disease, human fibrotic lung disease (e.g., obliterative bronchiolitis, idiopathic pulmonary fibrosis, pulmonary fibrosis, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, moderate to severe asthma and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport syndrome, HIV-associated nephropathy, polycystic kidney disease, Fabry's disease, diabetic nephropathy, chronic glomerulonephritis, nephritis associated with systemic lupus, and the like), progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's ophthalmopathy, diabetic retinopathy, glaucoma, Peyronie's disease, penis fibrosis, urethrostenosis after cystoscope, inner accretion after surgery, scarring, myelofibrosis, idiopathic retroperitoneal fibrosis, peritoneal fibrosis, drug induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), stromal cell tumors, mucositis, fibrosis induced by chemical or environmental insult (e.g., cancer chemotherapy, pesticides, radiation (e.g., cancer radiotherapy), and the like), and the like.

In some embodiments, the fibrotic disorder is selected from systemic or local scleroderma, keloids, hypertrophic scars, atherosclerosis, restenosis, pulmonary inflammation and fibrosis, idiopathic pulmonary fibrosis, liver cirrhosis, fibrosis as a result of chronic hepatitis B or C infection, fibrosis as a result of alcoholic disease, kidney disease, heart disease resulting from scar tissue, macular degeneration, retinal and vitreal retinopathy, pemphigoid, fibrosis associated with tumors, and fibrosis associated with aging. In some embodiments, the fibrosis related disorder results from chemotherapeutic drugs, radiation-induced fibrosis, and injuries and burns.

The disclosed methods to treat diseases and disorders characterized by excessive fibrosis formation and maintenance may also be used to suppress or inhibit inappropriate fibrosis formation. For example, they may treat or prevent a condition occurring in the liver, kidney, lung, heart and pericardium, eye, skin, mouth, pancreas, gastrointestinal tract, peritoneum, spleen, brain, breast, bone marrow, bone, muscles, tendons, genitourinary, a tumor, or a wound.

Generally, they may treat or prevent fibrosis related disorders resulting from conditions, including but not limited to, rheumatoid arthritis, lupus, pathogenic fibrosis, fibrosing disease, fibrotic lesions such as those formed after Schisto Soma japonicum infection, radiation damage, autoimmune diseases, Lyme disease, chemotherapy-induced fibrosis, HIV- or infection-induced focal sclerosis, failed back syndrome due to spinal surgery scarring, abdominal adhesion, post surgery scarring, and fibrocystic formations.

Specifically, in the liver, disclosed agents may treat or prevent fibrosis resulting from conditions including, but not limited to, alcohol, drug, and/or chemically induced cirrhosis, ischemia reperfusion, injury after hepatic transplant, necrotizing hepatitis, hepatitis B, hepatitis C, primary biliary cirrhosis, and primary sclerosing cholangitis.

Relating to the kidney, they may treat or prevent fibrosis resulting from conditions including, but not limited to, proliferative and sclerosing glomerulonephritis, nephrogenic fibrosing dermopathy, diabetic nephropathy, renal tubulointerstitial fibrosis, and focal segmental glomerulosclerosis.

Relating to the lung, they may treat or prevent fibrosis resulting from conditions including but not limited to pulmonary interstitial fibrosis, drug-induced sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema. Other causes of fibrosis include viral infection, e.g., SARS-CoV-2 infection.

Relating to the heart and/or pericardium, they may treat or prevent fibrosis resulting from conditions including, but not limited to, myocardial fibrosis, atherosclerosis, coronary artery restenosis, congestive cardiomyopathy, heart failure, and other post-ischemic conditions.

Relating to the eye, they may treat or prevent fibrosis resulting from conditions including but not limited to exopthalmos of Grave's disease, proliferative vitreoretinopa thy, anterior capsule cataract, corneal fibrosis, corneal scarring due to surgery, trabeculectomy-induced fibrosis, and other eye fibrosis.

Relating to the skin, they may treat or prevent fibrosis resulting from conditions including, but not limited to, Depuytren's contracture, scleroderma, keloid scarring, psoriasis, hypertrophic scarring due to burns, and psuedoscleroderma caused by spinal cord injury.

Relating to the mouth, they may treat or prevent fibrosis resulting from conditions including, but not limited to, periodontal disease scarring and gingival hypertrophy secondary to drugs.

Relating to the pancreas, they may treat or prevent fibrosis resulting from conditions including, but not limited to, pancreatic fibrosis, stromal remodeling pancreatitis, and stromal fibrosis.

Relating to the gastrointestinal tract, they may treat or prevent fibrosis resulting from conditions including but not limited to collagenous colitis, villous atrophy, cryphyperplasia, polyp formation, fibrosis of Crohn's disease, and healing gastric ulcer.

Relating to the brain, they may treat or prevent fibrosis resulting from conditions including, but not limited to, glial scar tissue.

Relating to the breast, they may treat or prevent fibrosis resulting from conditions including, but not limited to, fibrocystic disease and desmoplastic reaction to breast cancer.

Relating to the bone marrow, they may treat or prevent fibrosis resulting from conditions including, but not limited to, fibrosis in myelodysplasia and neoplastic diseases.

Relating to the bone, they may treat or prevent fibrosis resulting from conditions including, but not limited to, rheumatoid pannus formation.

Relating to the genitourinary system, they may treat or prevent fibrosis resulting from conditions including, but not limited to, endometriosis, uterine fibroids, and ovarian fibroids.

Disclosed herein are methods of treating or preventing a fibrotic disease or disorder, including fibroproliferative disorders, comprising administering to a subject in need thereof one or more agents. In some embodiments, the agent is selected from one of the following structures:

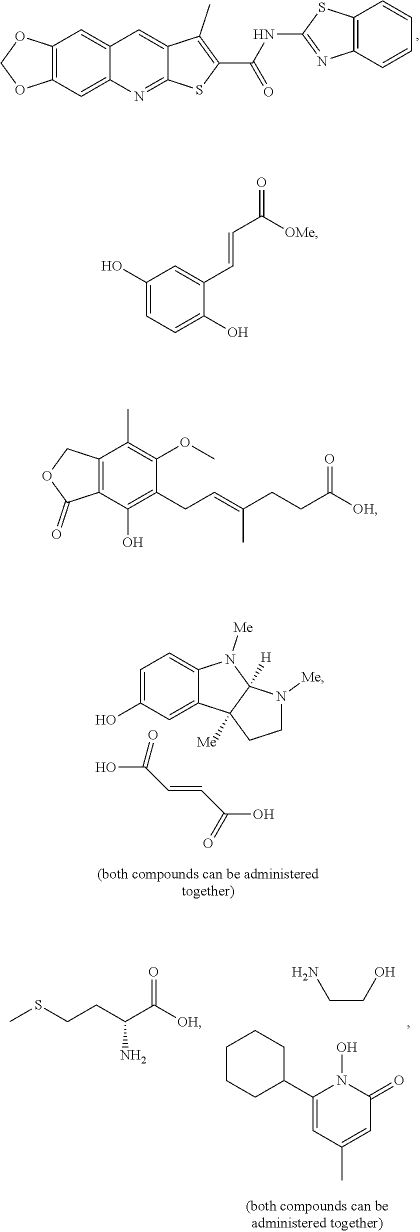

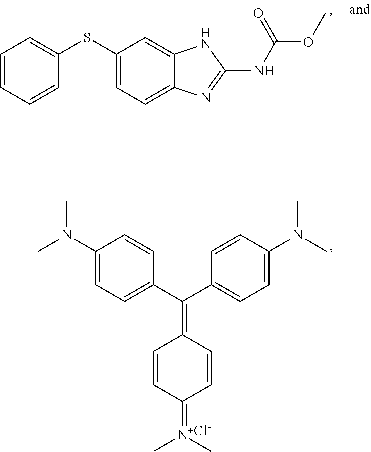

or a pharmaceutically acceptable salt thereof. In certain preferred embodiments, the agent is

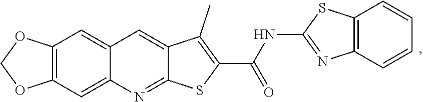 [SMILES: Cc1c(C(=O)Nc2nc3ccccc3s2)sc2nc3cc4c(cc3cc12)OCO4]

also known as AA5. In other preferred embodiments, the agent is

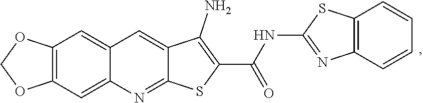 [SMILES: Nc1c(C(=O)Nc2nc3ccccc3s2)sc2nc3cc4c(cc3cc12)OCO4]

also known as AA5-SAR2. In certain embodiments, the biologically active agent in

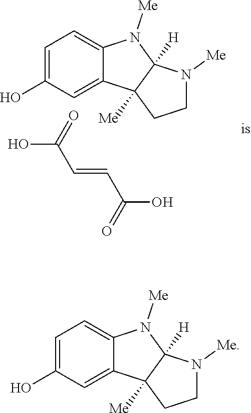

In certain embodiments, the biologically active agent in

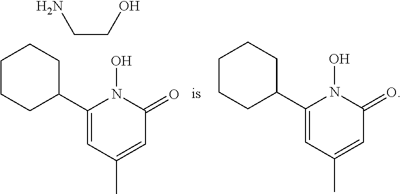

### In Vitro Model of Fibrosis

The accumulation of ECM triggers progressive organ remodeling and therefore organ dysfunction. Often this fibrotic process is driven by metabolic and inflammatory diseases that result in organ injury and perpetuate the fibrosis, which is driven by mesenchymal cells. Many different diseases all result in the same fibrotic response in different organs such as the liver, kidney, lung and skin, which suggests a common disease pathogenesis. However, it has been challenging to develop relevant human models of progressive fibrosis, mainly due to the challenges in reproducing persistent inflammation and cellular plasticity that precedes tissue remodeling and fibrosis. Further, for nearly three decades, more than 90% of drug candidates identified to be effective in animal models of fibrosis (e.g. bleomycin-induced lung fibrosis) have failed in clinical trials.

Disclosed herein is an in vitro human model that recapitulates the common inflammation-driven progressive fibrosis seen across organs. The unique response of induced pluripotent stem cells (iPSCs) differentiated to multiple different cell types and cultured on a stiff polyacrylamide hydrogel reproduces the molecular and cellular pathways found in progressive fibrotic disorders. This model of progressive fibrosis is amenable to drug screening, which led to identification of the agents discussed above. This model was developed beginning with identification of iPSCs as a resource for developing several cell types for fibrosis modeling.

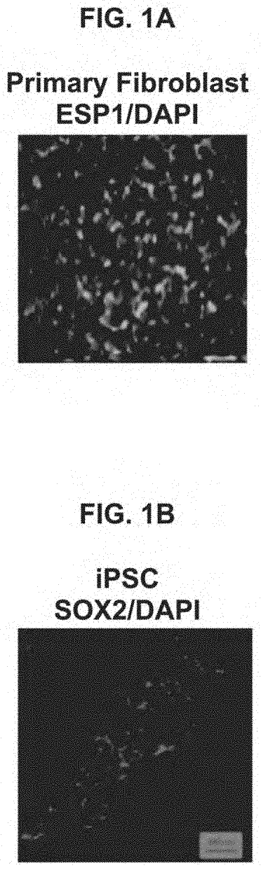

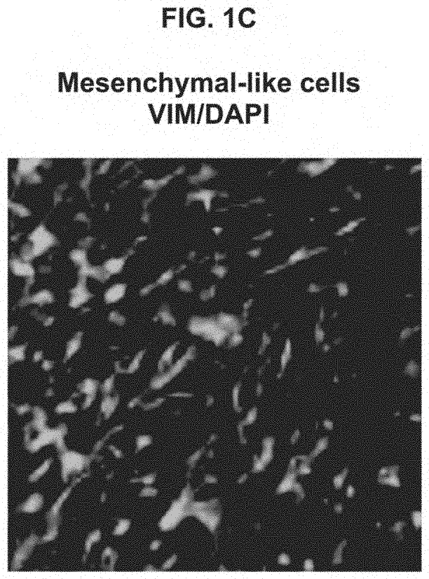

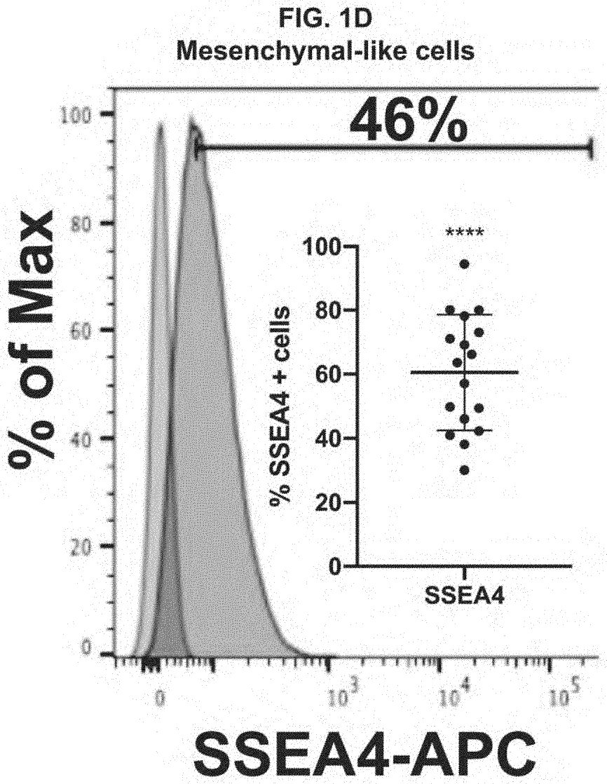

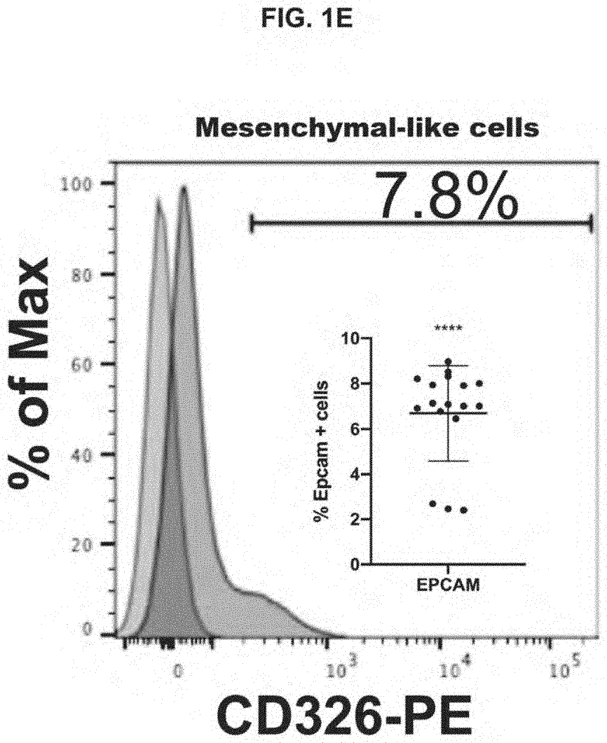

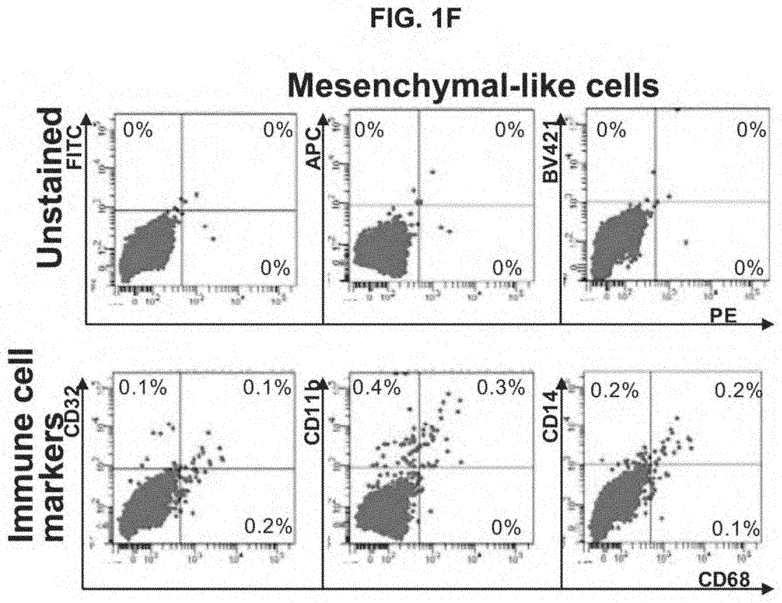

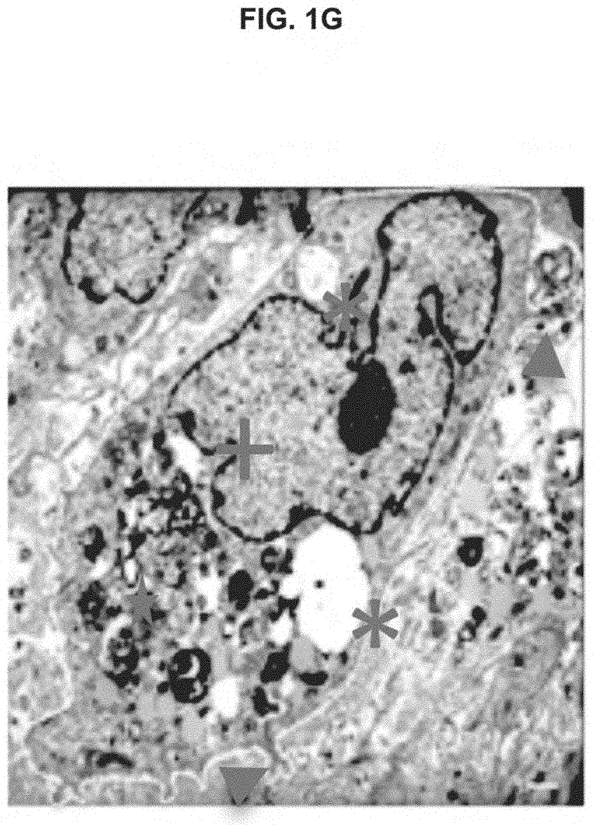

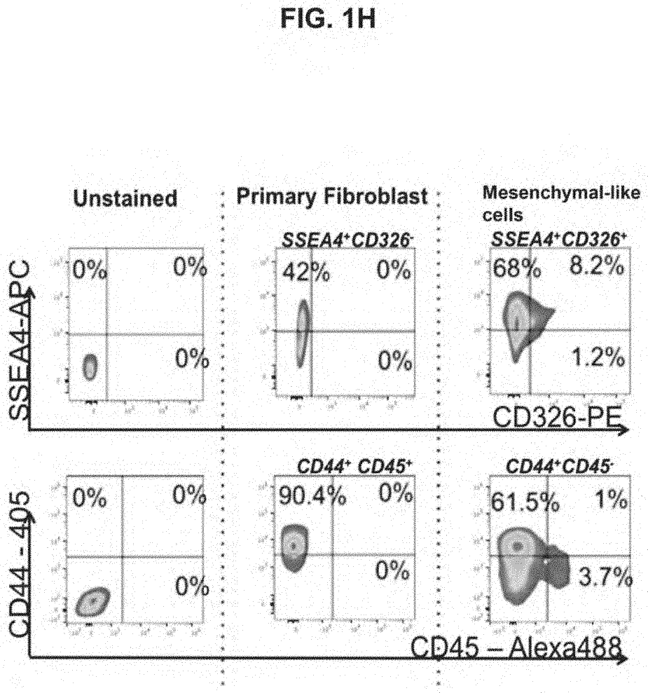

Progressive fibrosis can occur in any organ and arises from the cumulative effect of aberrant wound repair involving multiple cell types, including fibroblasts, epithelial cells, and immune cells responding to various mechanical and chemical stimuli. iPSCs were used to model the complex phenotype of progressive fibrosis. Every tissue in the body is capable of a wound healing response that involves a scarring phase. Reprogramming human somatic cells to iPSCs from any source and disease state leads to erasure of the existing somatic epigenetic memory. Cell sources used in developing iPSCs for the present model included dermal and lung fibroblasts, and peripheral blood mononuclear cells (PBMCs), which were differentiated them into multiple different cell types critical for modeling fibrosis (See, FIG. 1A-H).

The differentiated cells were comprised of over 90% mesenchymal-like cells, as determined by their morphology in cell culture and expression of mesenchymal markers such as Vimentin (VIM) (FIG. 1C). Over 30% (mean+s.e.m, 60.5%+4.3) of the mesenchymal-like cells showed expression of SSEA4 (FIG. 1D), a marker associated with a fibrosis-initiating cell population in lung. A subpopulation of mesenchymal-like cells that expressed the epithelial cell marker, CD326 (6.7%+0.5, mean+s.e.m,) (FIG. 1E). There was also a separate small population of cells (4.9%+0.64, mean+s.e.m,) that expressed the immune cell markers CD45, CD32, CD11b, CD68 or CD14 (FIGS. 1F,H). Cells with the classic features of macrophages such as heterochromatin, vacuolated cytoplasm, microvilli and whorls of phagocytosed matter were found in low numbers within the differentiated cells by transmission electron microscopy (TEM) of the cells in the cultures (FIG. 1G).

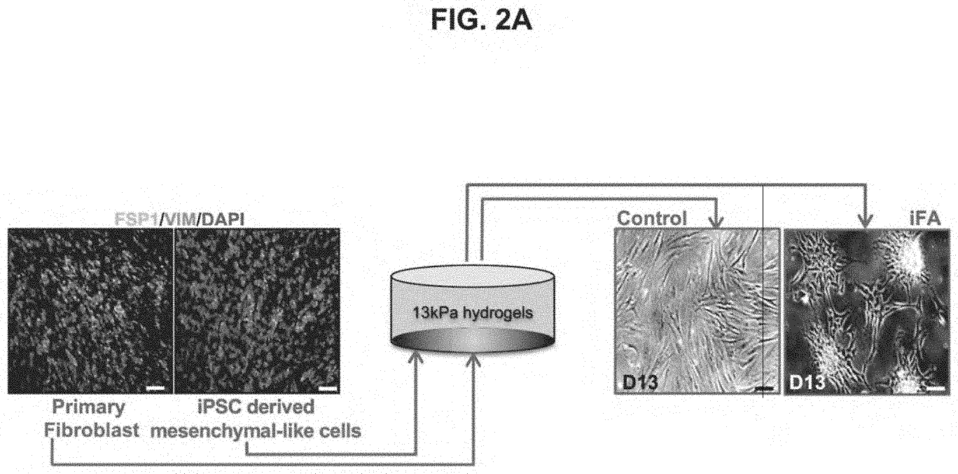

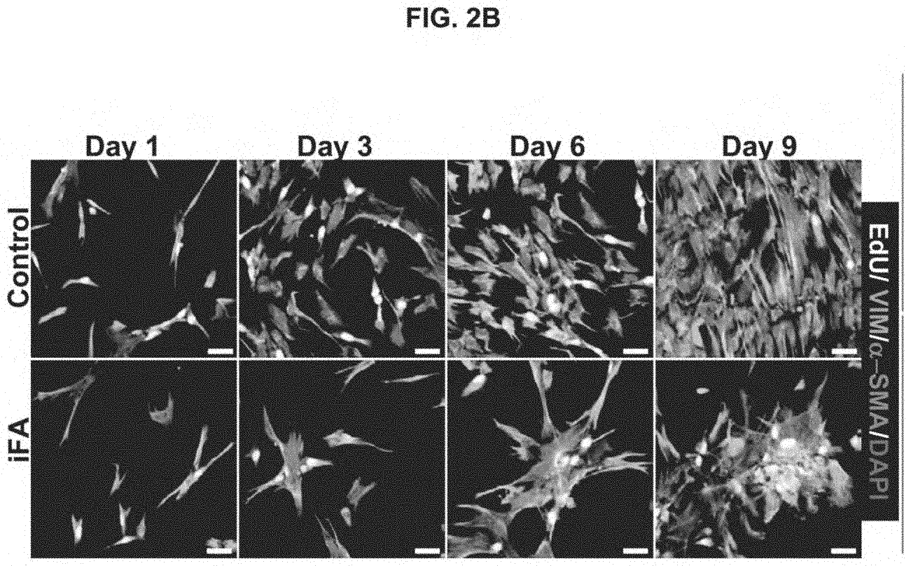

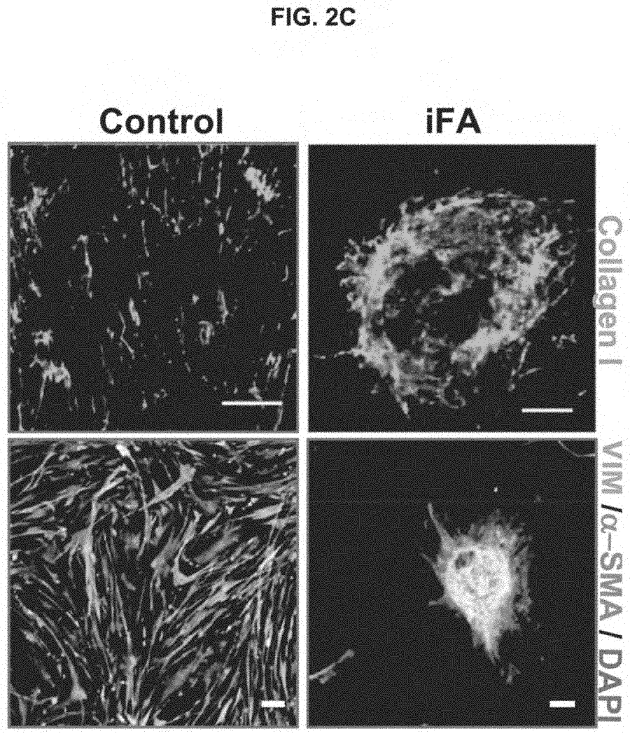

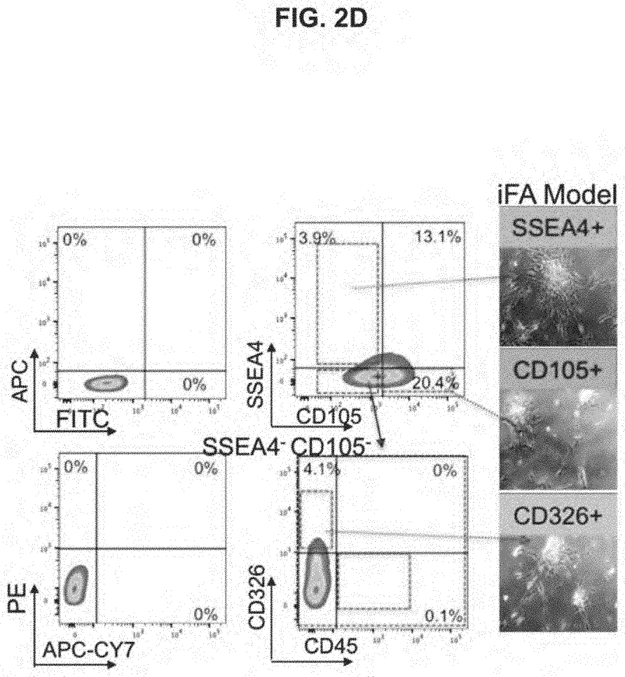

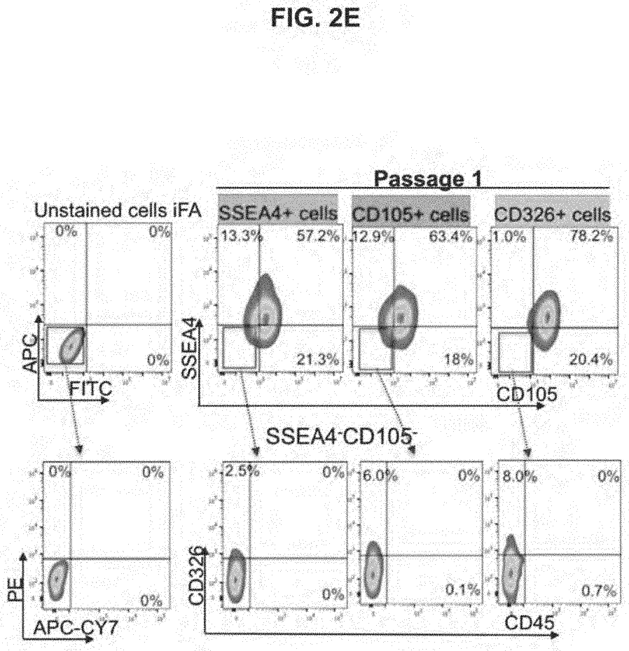

The mesenchymal-like cells were isolated and cultured on polyacrylamide-based hydrogels at 13 kPa, a stiffness that approximates that of a fibrotic organ. Hydrogels were functionalized with benzoquinone and coated with 0.1% gelatin. Cellular controls for this model were primary fibroblasts obtained from the same anatomical sites as the site from which the mesenchymal-like cells were derived (parent primary fibroblast. (FIG. 2A). The mesenchymal-like cells shared similar expression patterns of SSEA4 and CD44 with their parent primary fibroblasts (FIG. 1H). However, the mesenchymal-like cells also expressed CD326 and CD45, which the parent primary fibroblasts lacked (FIG. 1H).

During a 13-day culture period on 13 kPa hydrogels, the mesenchymal-like cells failed to form a monolayer like that of the primary fibroblasts cultured under the same condition. Instead, the mesenchymal-like cells were highly proliferative and formed dense, progressively enlarging cellular aggregates (FIGS. 2A,B). the mesenchymal-like cells continued to proliferate as aggregates (FIG. 2B), Consistently, these aggregates revealed increased gene expression and protein levels of Collagen I, α-SMA and TGF-β when compared to the primary fibroblasts cultured under the same conditions (FIG. 2C).

The cultured mesenchymal-like cells in the model share many of the characteristics of the induced fibroblastic activation (iFA) phenotype that is classically seen in organ fibrosis. The iFA phenotype was consistently observed in all mesenchymal-like differentiated cell cultures (n=17 patient samples)(Table S1), while primary fibroblasts showed no fibroblast activation phenotype irrespective of the source of tissue from which they were isolated.

The mesenchymal-like cells demonstrated plasticity as shown in FIGS. 2D and 2E. The pathological effects of progressive fibrosis are associated with cell plasticity, which plays a major role in the phenotypic transitions in cell populations that contribute to tissue remodeling in organ fibrosis. These phenotypic transitions are commonly seen as epithelial-to-mesenchymal transition and mesenchymal-to-epithelial transitions. Cell plasticity is also apparent from single-cell RNA sequencing studies from fibrotic lung where individual epithelial cells express markers of both distal lung and conducting airways, demonstrating undetermined cell types are a characteristic feature of fibrotic tissue. Thus, the plasticity of the model mesenchymal-like cells mimics that seen in human progressive fibrosis.

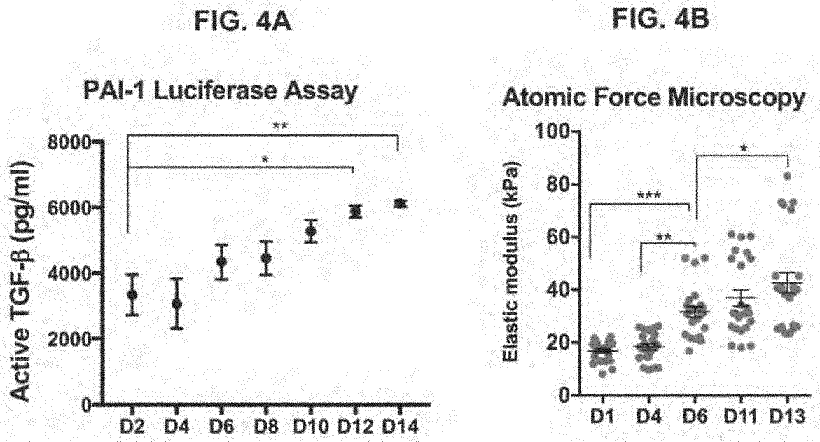

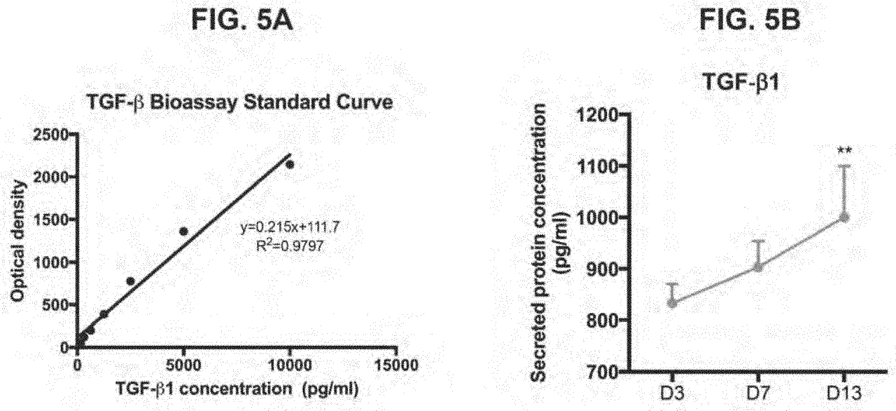

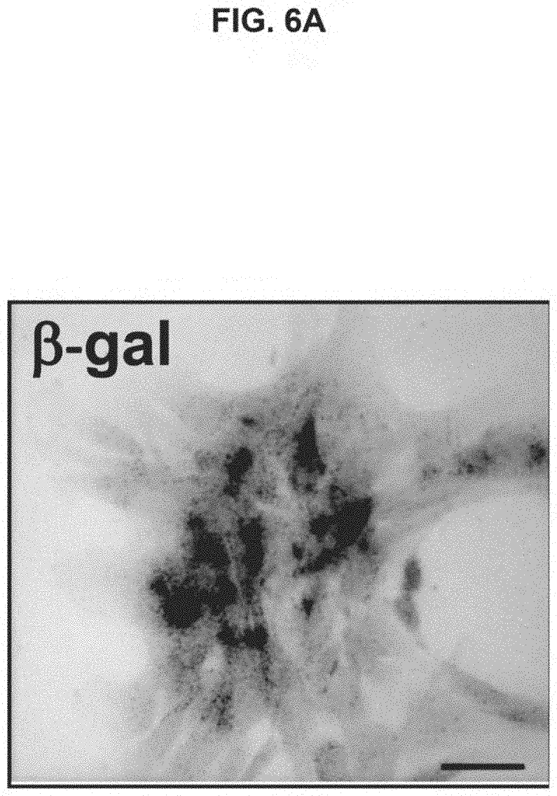

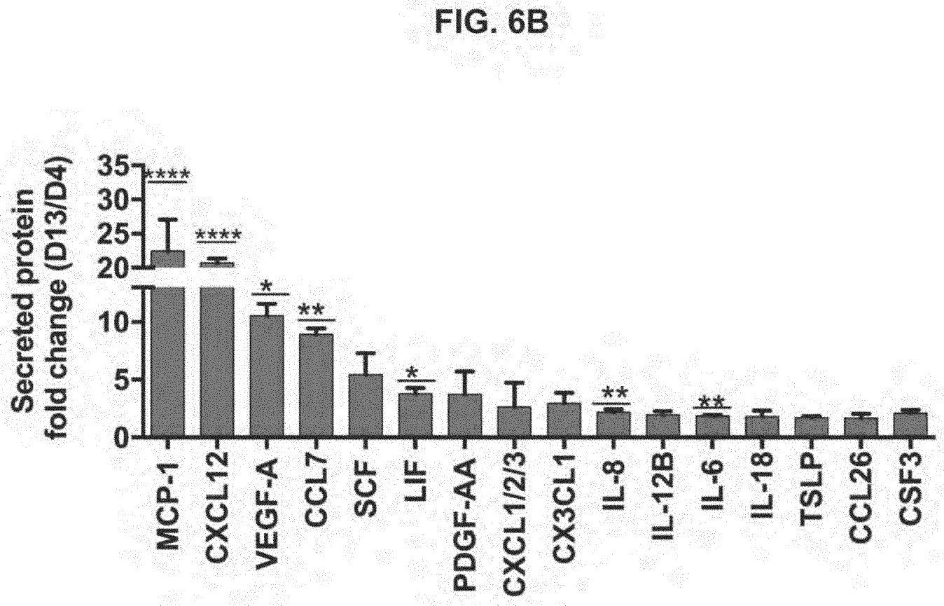

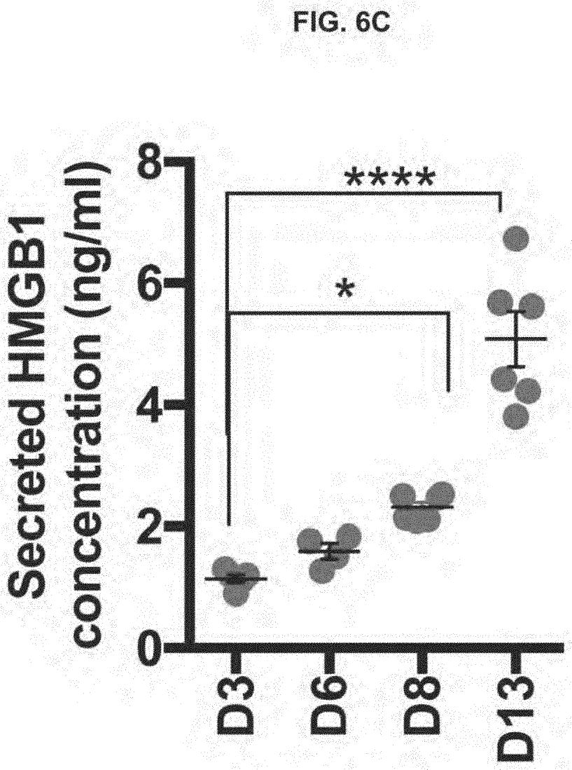

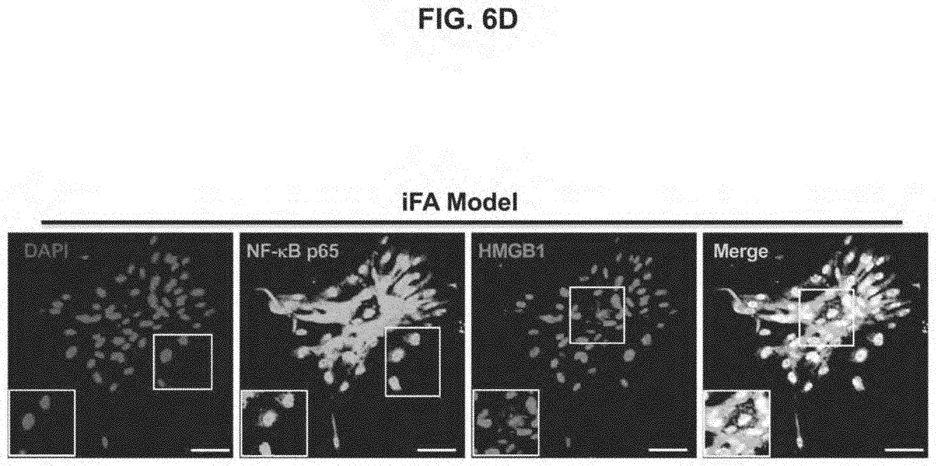

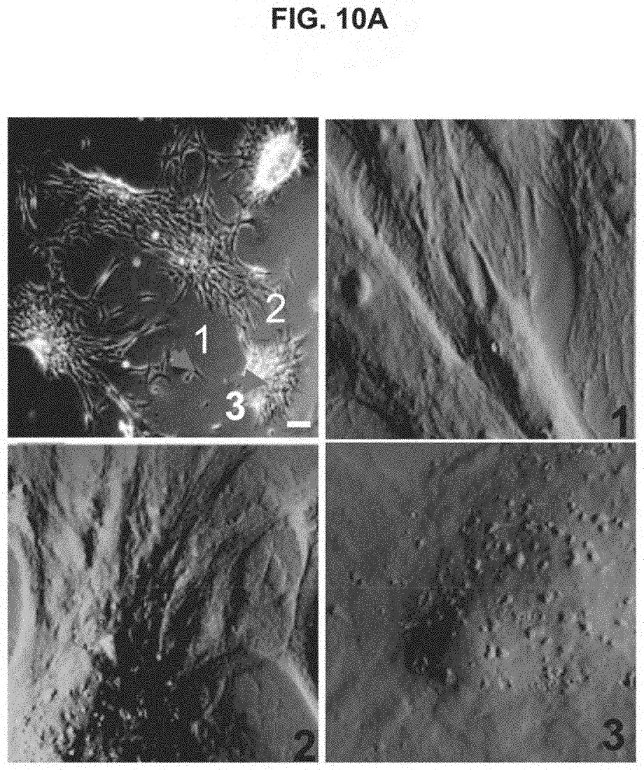

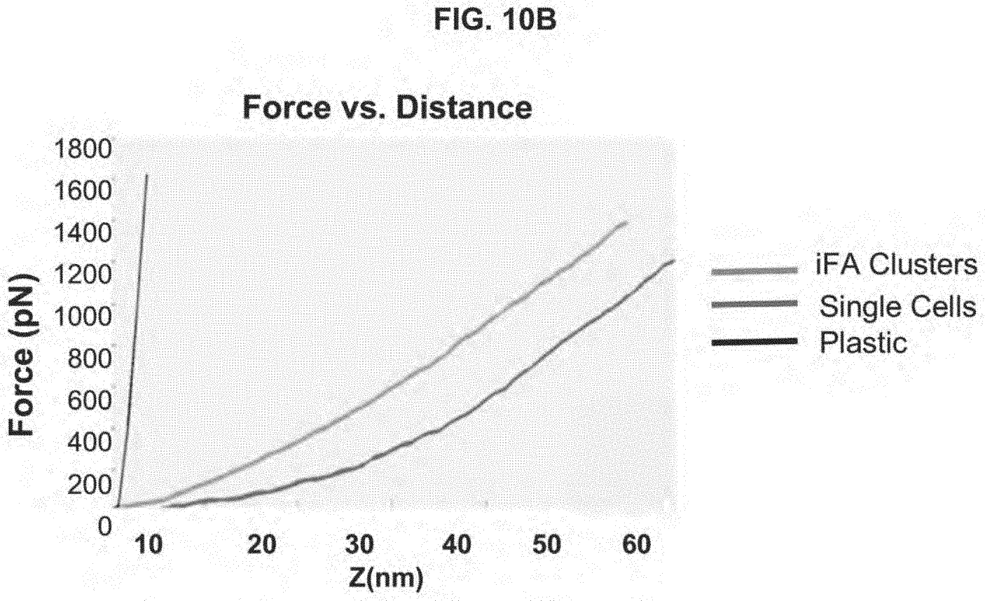

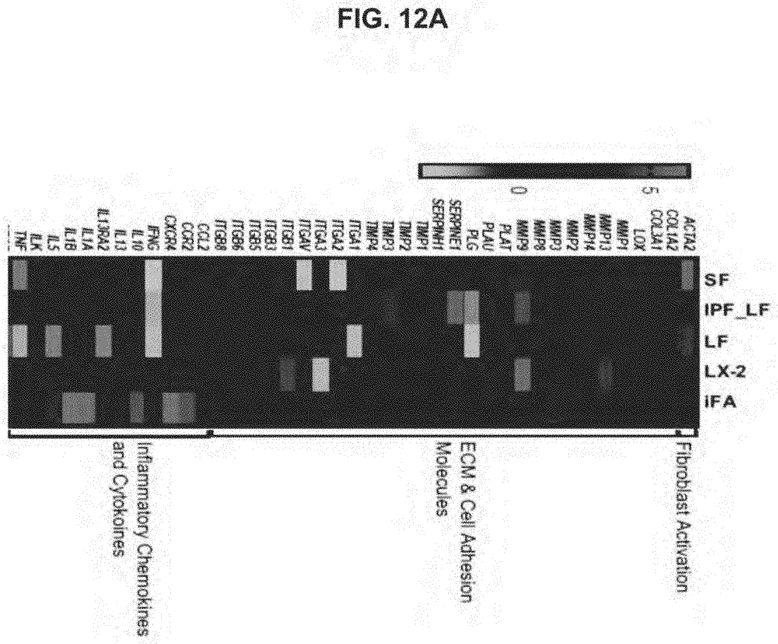

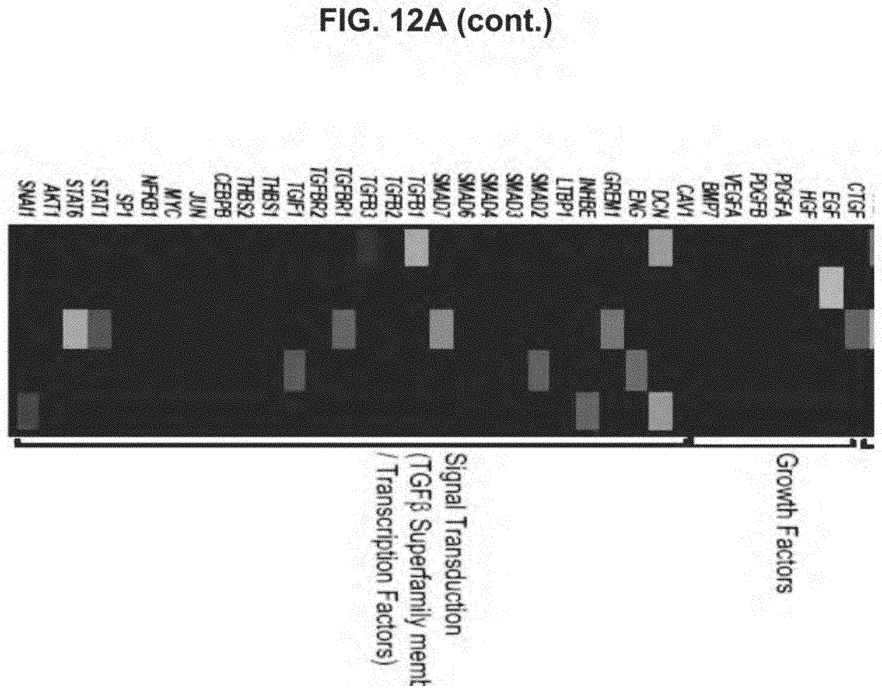

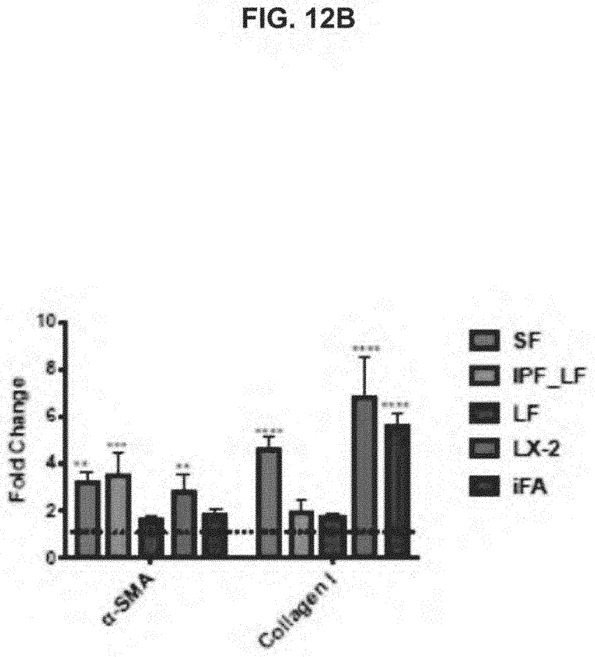

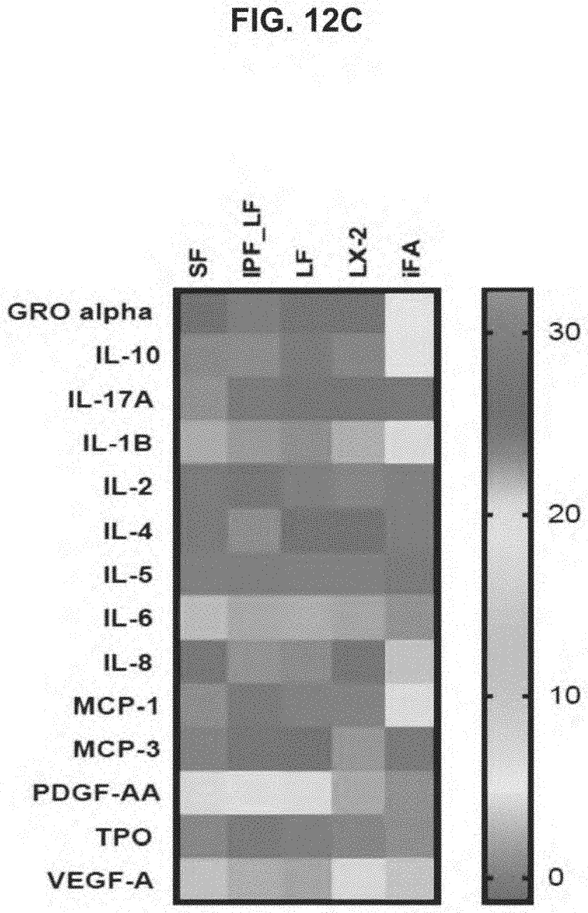

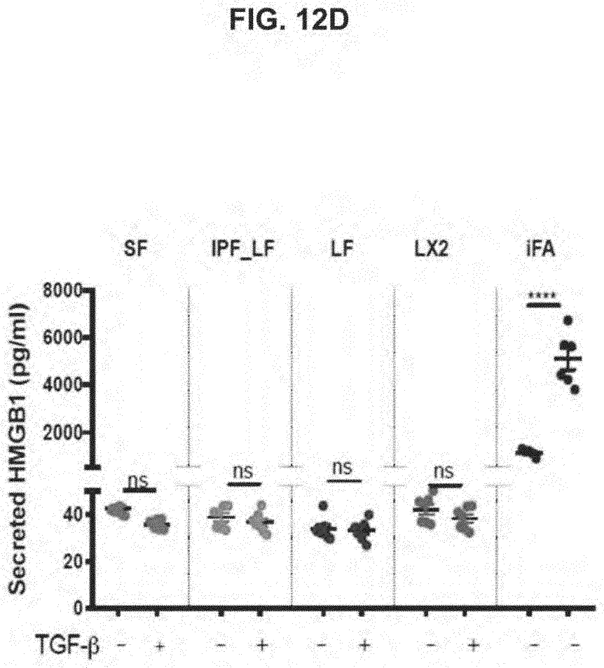

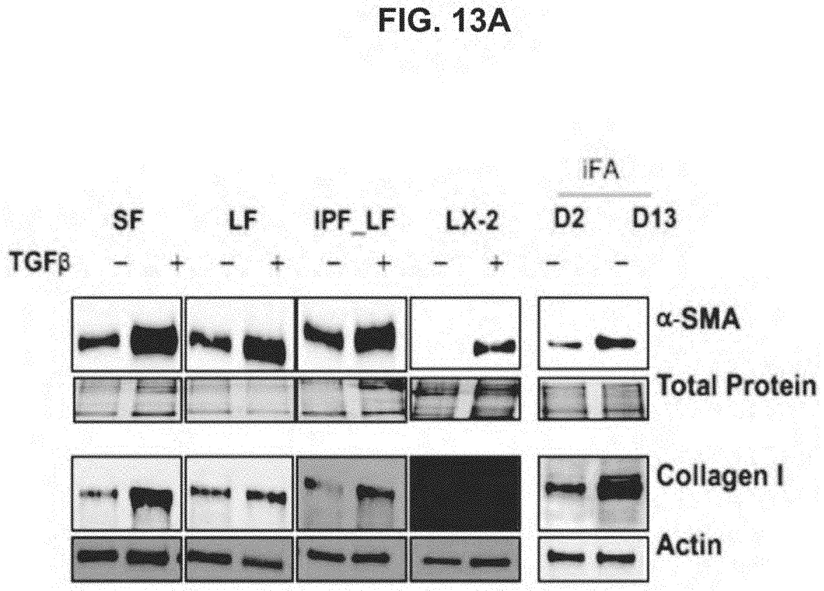

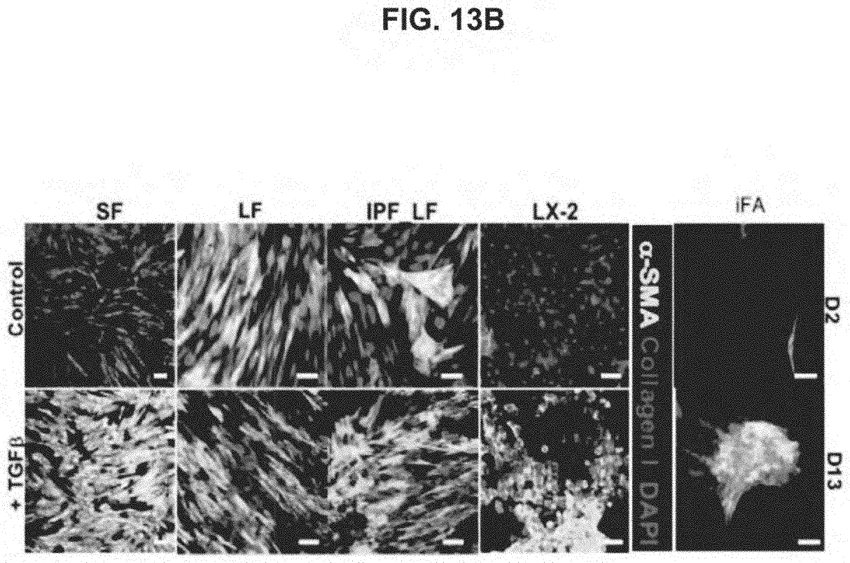

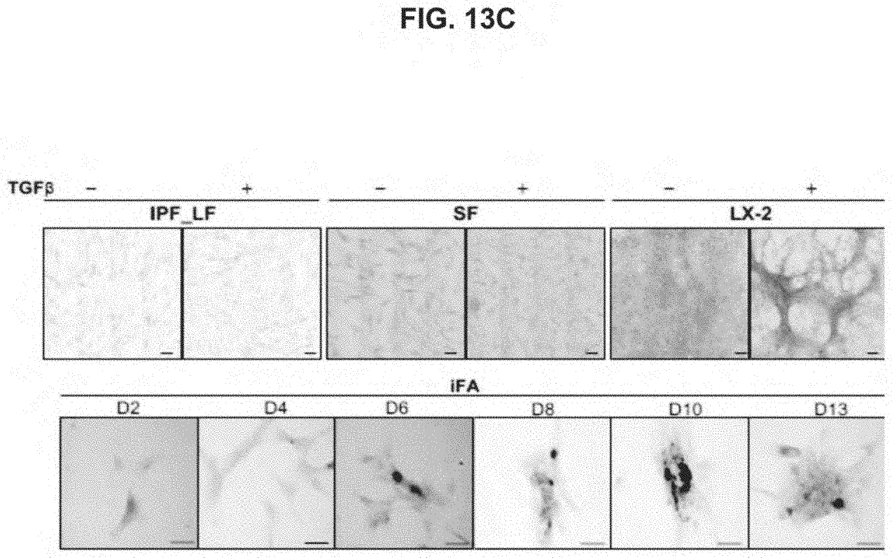

FIGS. 4A, 5A, and 5B depict further validation of the model by showing increasing amounts of active TGF-β over time. Parenchymal stiffness was evaluated and compared to levels in human fibrosis as shown in FIG. 4B and FIGS. 10A,B. Levels of other markers of fibrosis, such as cytokines, chemokines, and nuclear HMGB1 measured in FIG. 6B indicate consonance with that seen in human fibrotic tissue. Comparison of the disclosed model with known models of fibrosis included primary hepatic stellate cells (LX-2), primary fibroblasts from healthy (LF) and fibrotic lung (IPF_LF) and healthy skin (SF) that were exogenously treated with TGF-β for 48 hours are described in FIGS. 12A, B and 13A, B. The present model, unlike the other models, showed a higher inflammatory chemokine/cytokine, growth factor and TGF-β superfamily involved signal transduction signature. Significant levels of secreted cytokines/chemokines such as IL-6, IL-8, MCP-1 and VEGF-A and the DAMP molecule, HMGB1, were observed in the disclosed model, whereas the primary fibroblasts that were exogenously treated with TGF-β did not show a significant increase in expression of these proteins after treatment (FIGS. 12C,D). The level of senescent cells increased in this model but were in present in the known models (FIG. 13C). These data indicate the effectiveness of the present model to match the in vivo process of fibrosis.

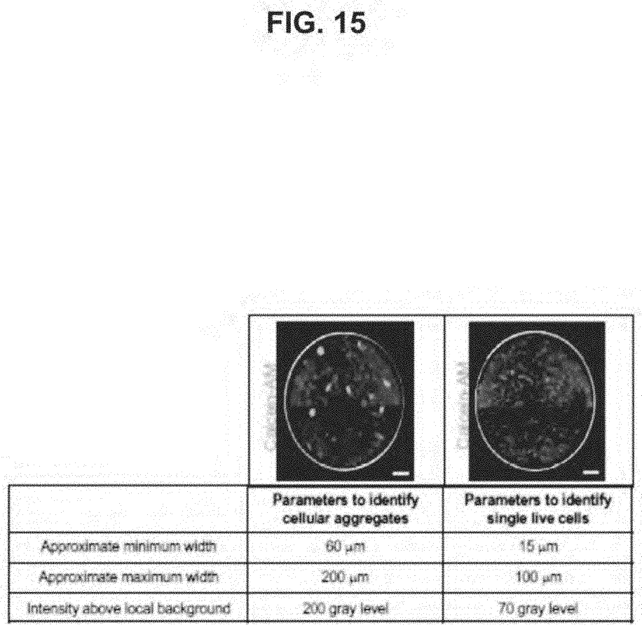

The present model was used in screening small molecule libraries to identify those with anti-fibrotic activity. A phenotypic assay was developed where parameters measured included cell size, fluorescence intensity of the iFA cellular aggregates, the shape factor of the cells/aggregates, and the cells' viability in the presence of viability dyes (FIG. 15). Anti-fibrotic activity was identified by determining if cells in the model did not form cellular aggregates and instead allow them to grow as a monolayer of viable cells with a spindle-shaped mesenchymal-like cell morphology. The model was subjected to compounds from in-house curated libraries of ˜17,000 small molecules at a concentration of 10 μM for 7 days. Wells with only DMSO were used as negative controls to assess drug efficacy for preventing the progression of the iFA phenotype

One hit compound was

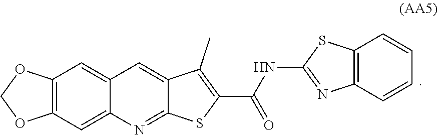 [SMILES: Cc1c(C(=O)Nc2nc3ccccc3s2)sc2nc3cc4c(cc3cc12)OCO4]

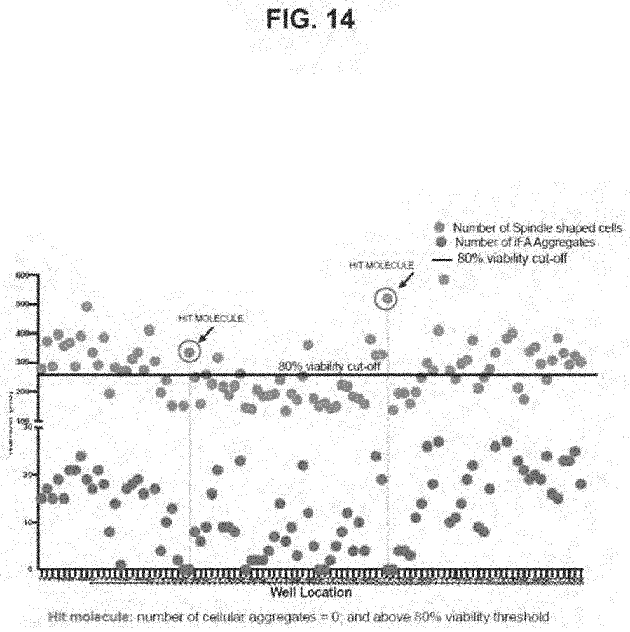

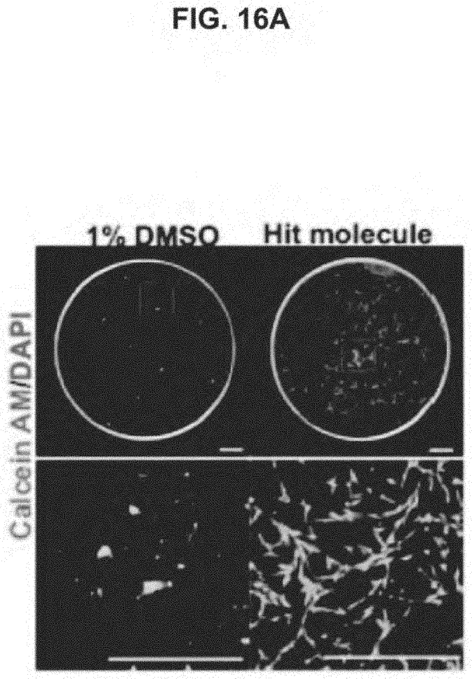

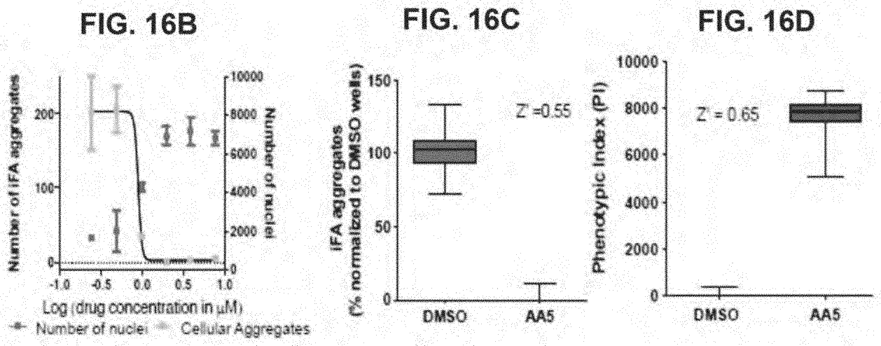

Compound AA5 was assayed for effects on cellular proliferation, tissue remodeling during repair, resolution of the fibrotic phenotype, and cell stiffness. AA5 was tested for its preventative effects and resolution of fibrosis effects as demonstrated in FIGS. 14, 16A,B and 17A,B. These experiments included transcriptomic analysis, RRHO analysis, hypergeometric overlap, differential gene expression analysis, functional cluster analysis, regulation of chemotaxis and activation of acute-phase proteins and cytokines, and upregulation of modulators of tissue repair. In all of these studies, the disclosed model confirmed the anti-fibrotic activity of AA5.

A phenotypic index was developed was calculated as follows: PI=(Area covered in the well)×(Number of nuclei)/Log (Number of iFA aggregates detected+5). Using the PI, Z′=0.65 was obtained which indicated a significant assay (FIG. 16C,D). The numerical value of 5 that is added to the iFA aggregates is roughly one standard deviation of the number of iFA aggregates found in an average well. For the iFA aggregate analysis, the signal to basal ratio (S/B) ratio was 59.3-fold, and the CV was 0.31 for the DMSO treated wells and 3.5 for the AA5 treated wells (FIG. 16C,D). For the PI analysis, the signal to basal ratio (S/B) ratio was 76-fold, and the CV was 0.63 for the DMSO treated wells and 0.11 for the AA5 treated wells (FIG. 16D).

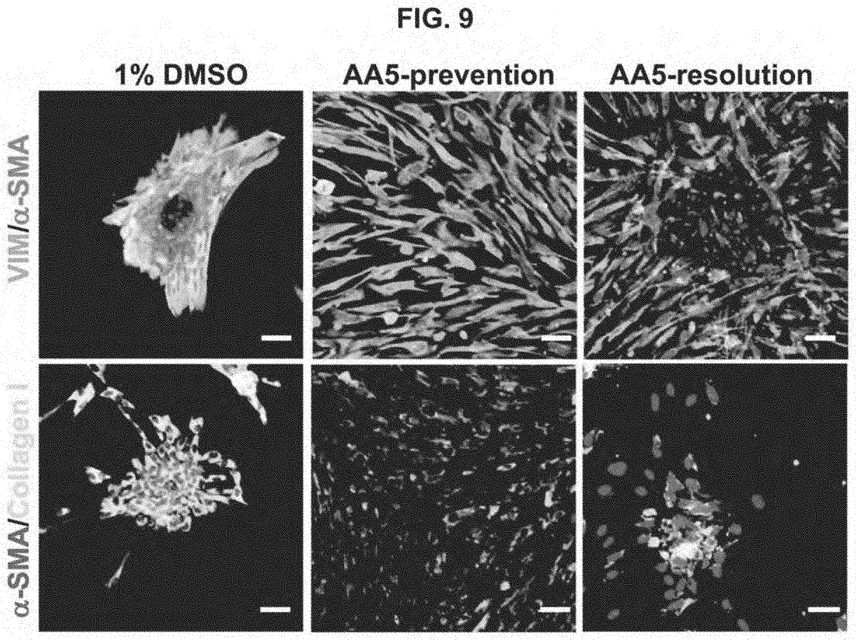

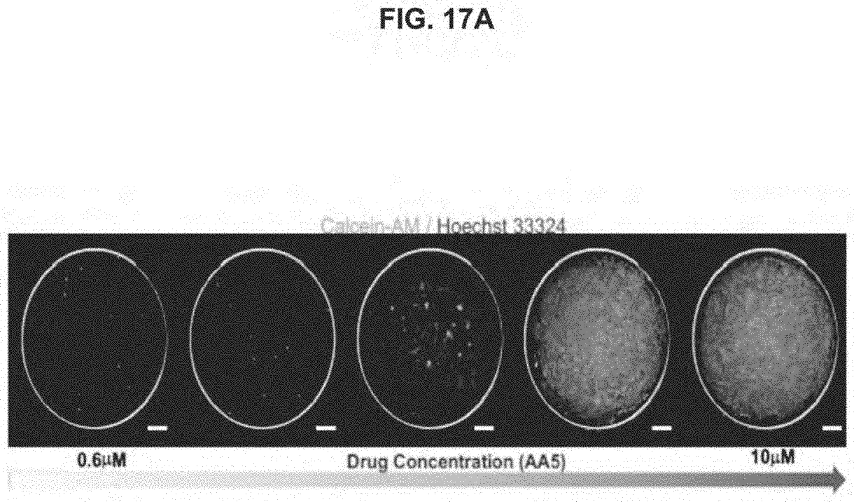

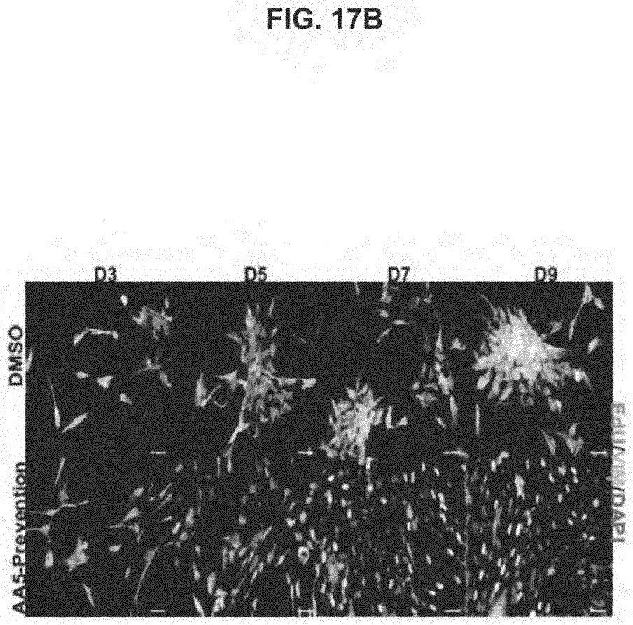

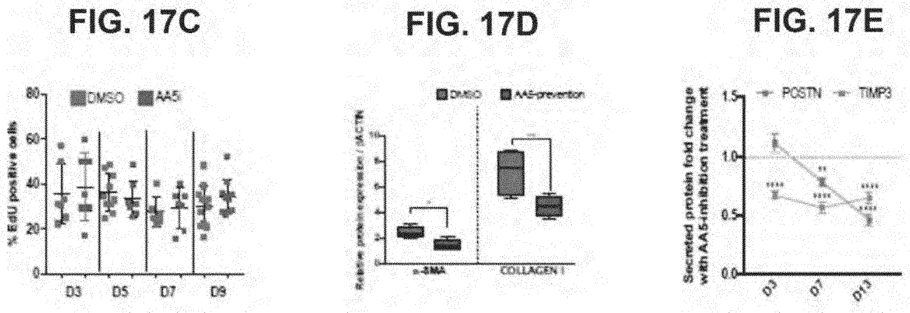

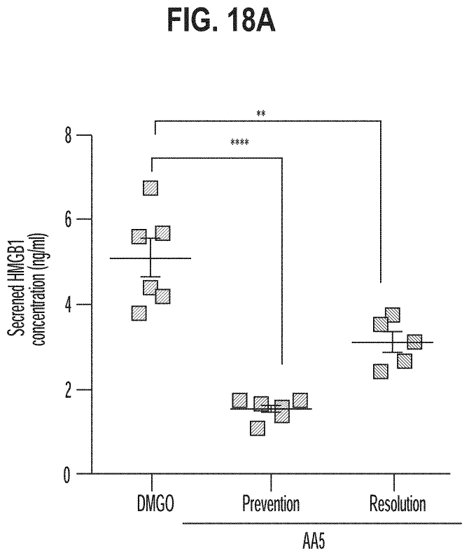

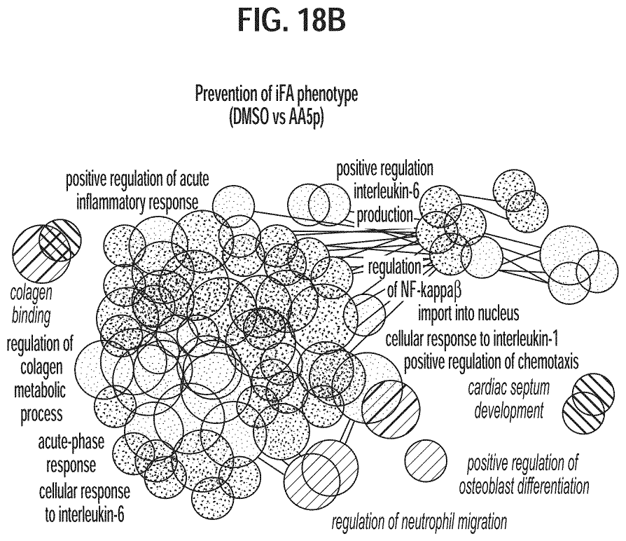

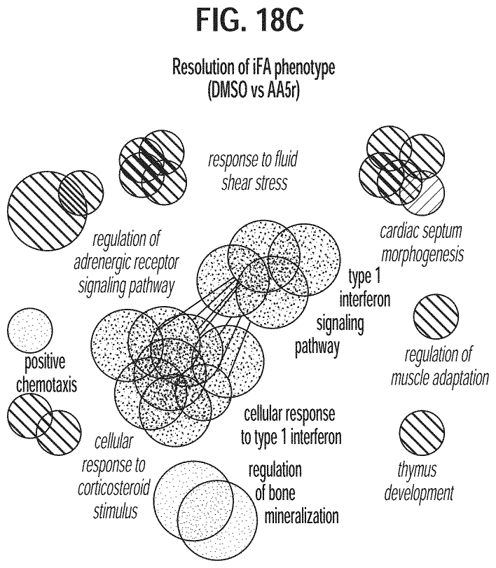

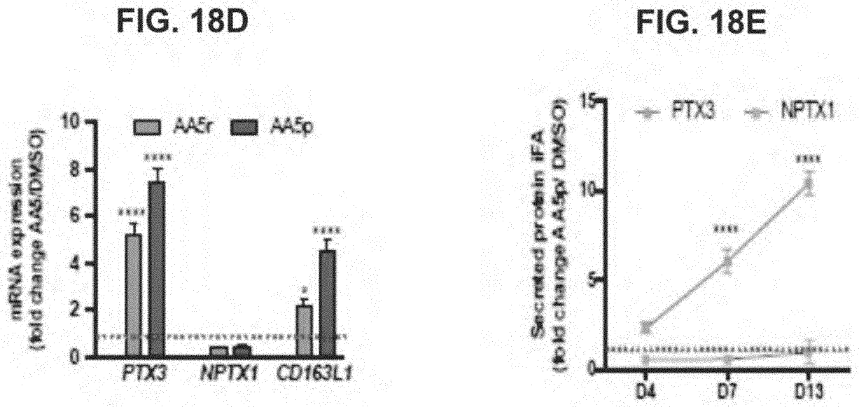

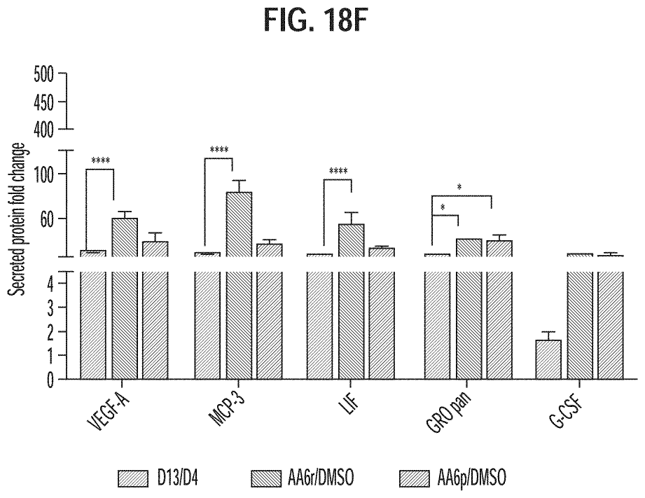

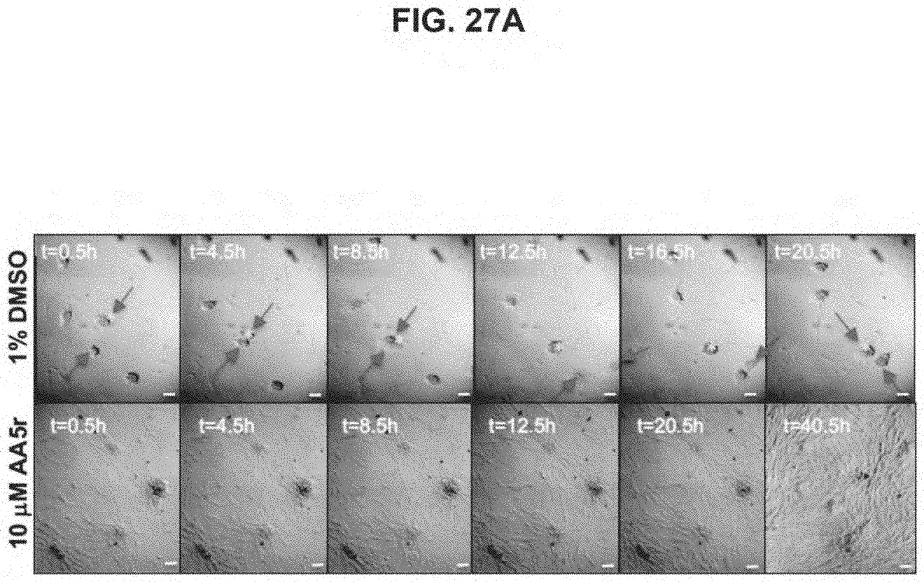

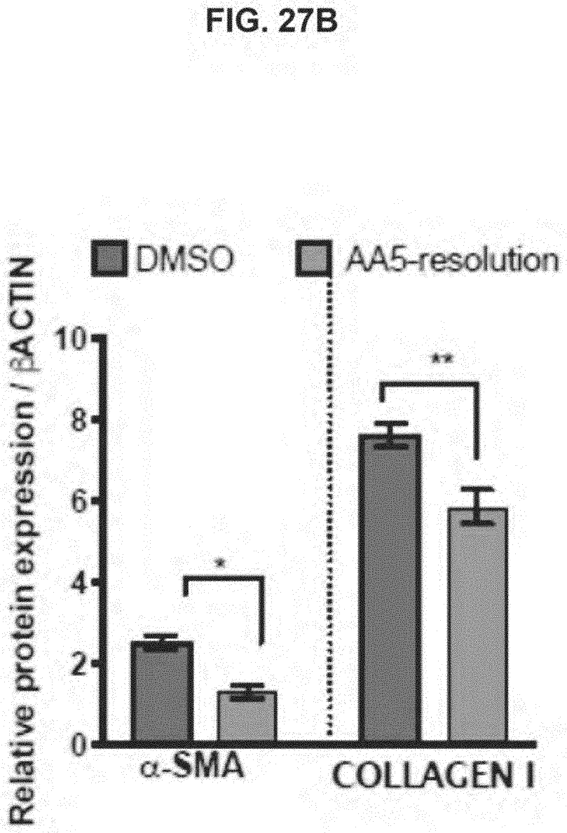

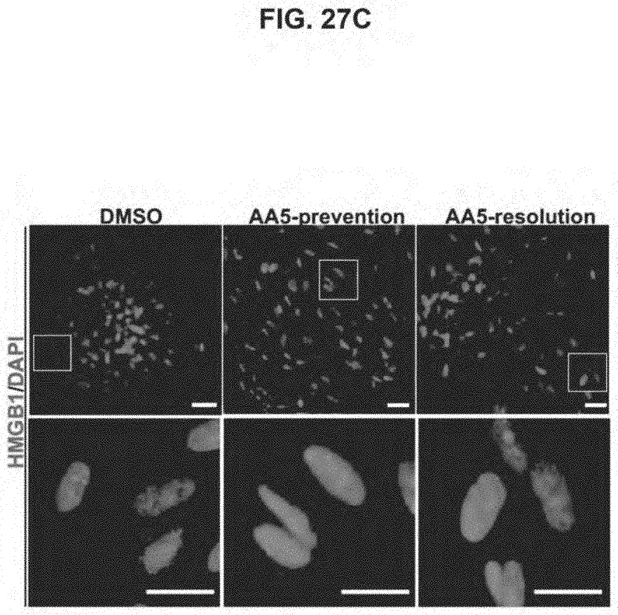

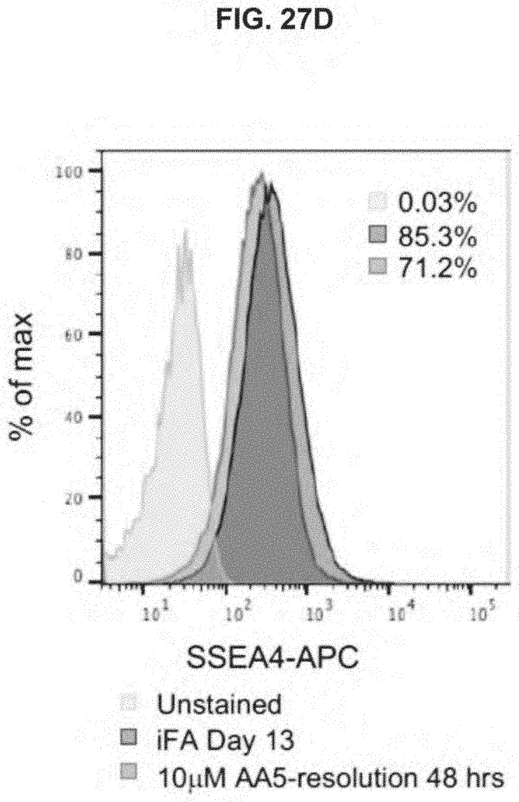

In evaluating the anti-fibrotic effect of AA5, testing AA5 treated cells showed no effect on proliferation (FIGS. 17B,C). However, down-regulation of the gene expression of the fibrotic markers α-SMA, Collagen I, TIMP-3 and POSTN in response to treatment with AA5 was further confirmed by immunoblotting, luminex and immunofluorescence analysis (FIGS. 17D,E). AA5 did resolve the iFA phenotype based on the reduction in levels of Collagen I and α-SMA (FIG. 9, FIGS. 27A,B). AA5 treatment significantly decreased cell stiffness (FIG. 27A). Additionally, secreted levels of HMGB1 and percentages of SSEA4+ cells were attenuated with AA5 treatment (FIG. 18A and FIGS. 27C,D).

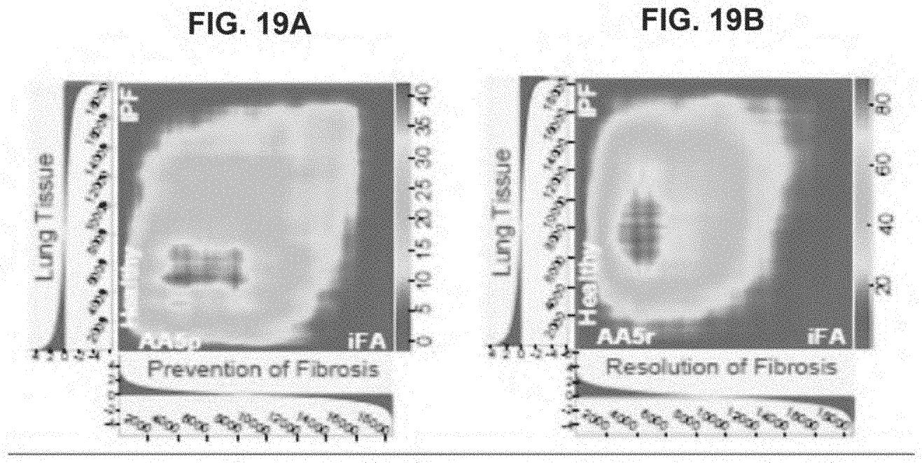

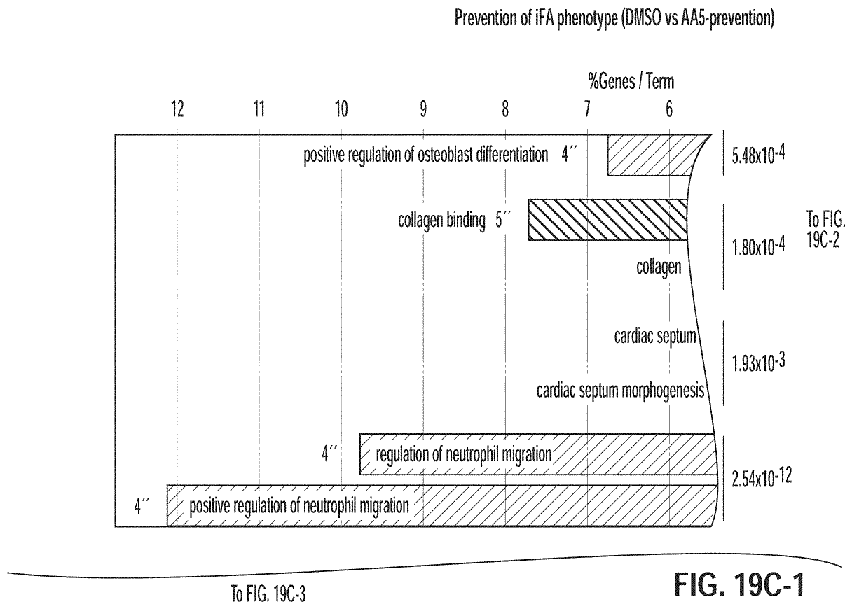

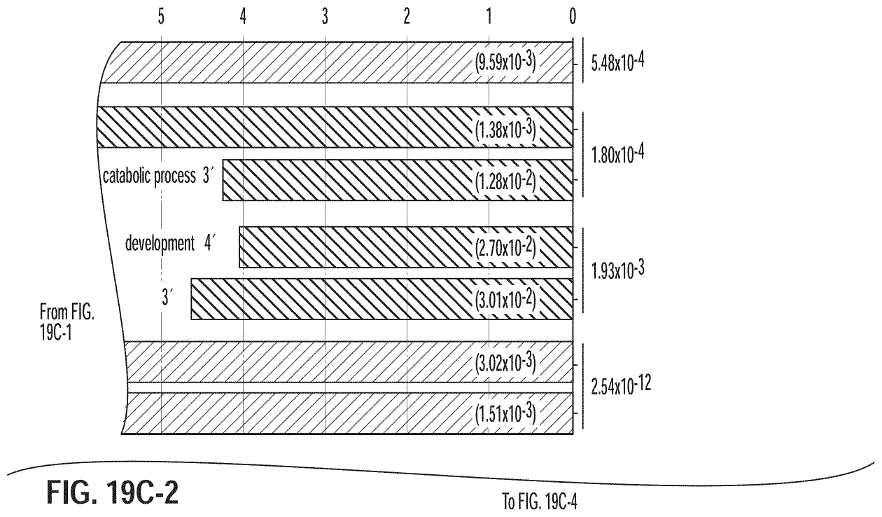

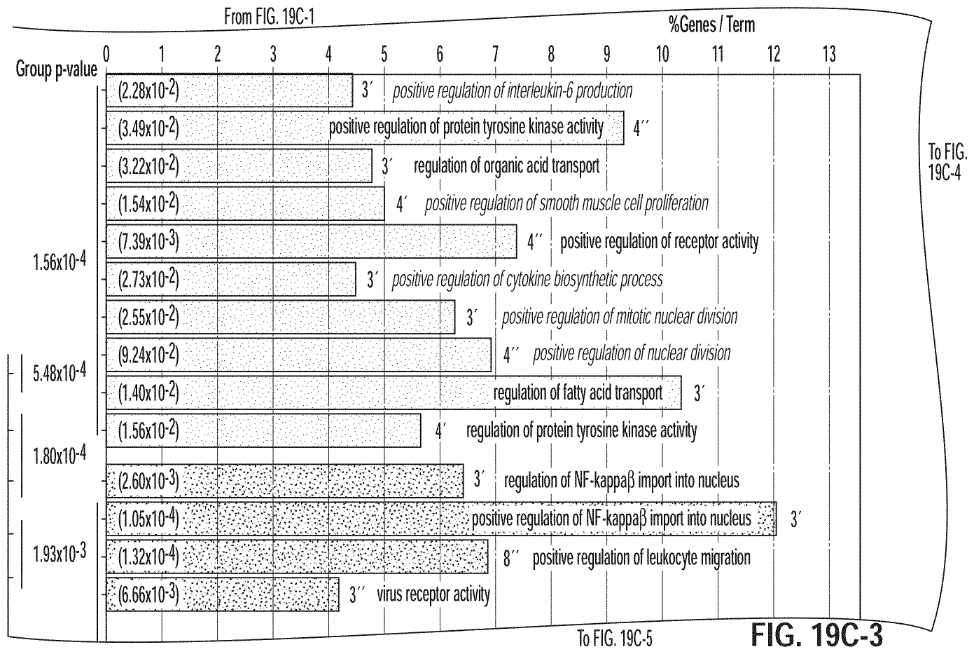

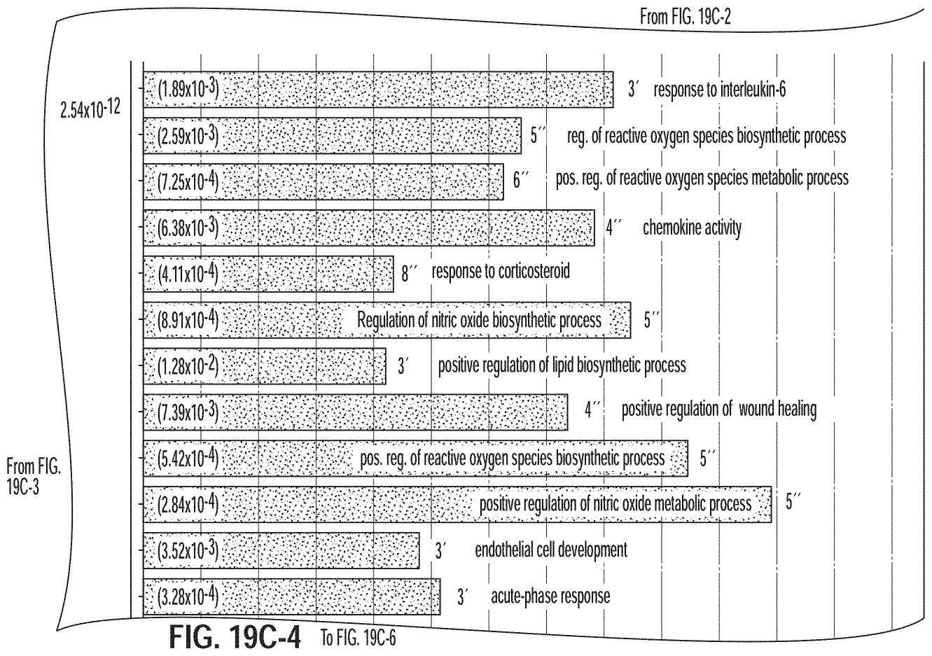

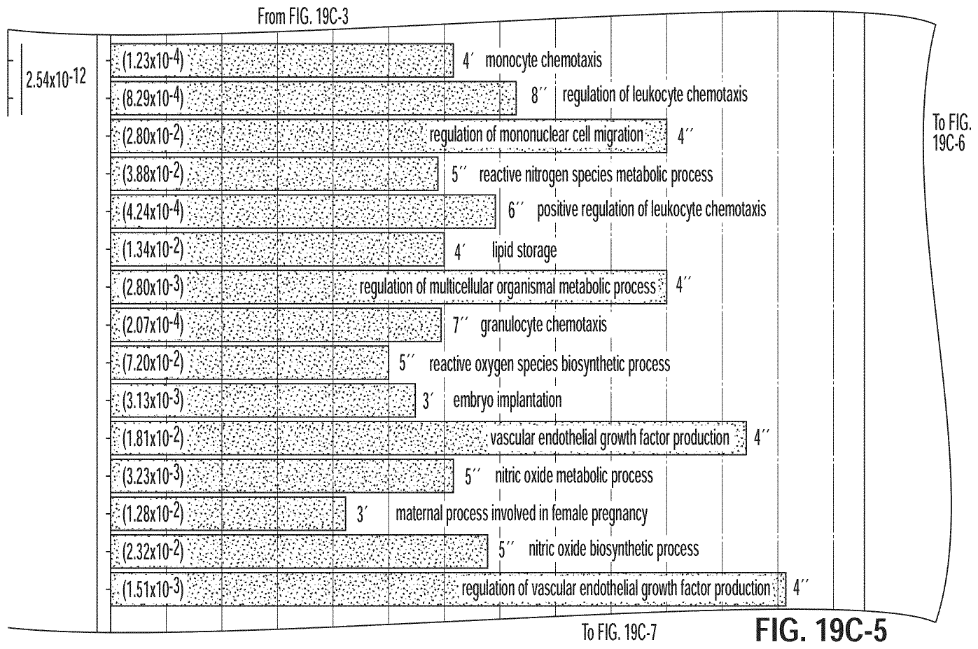

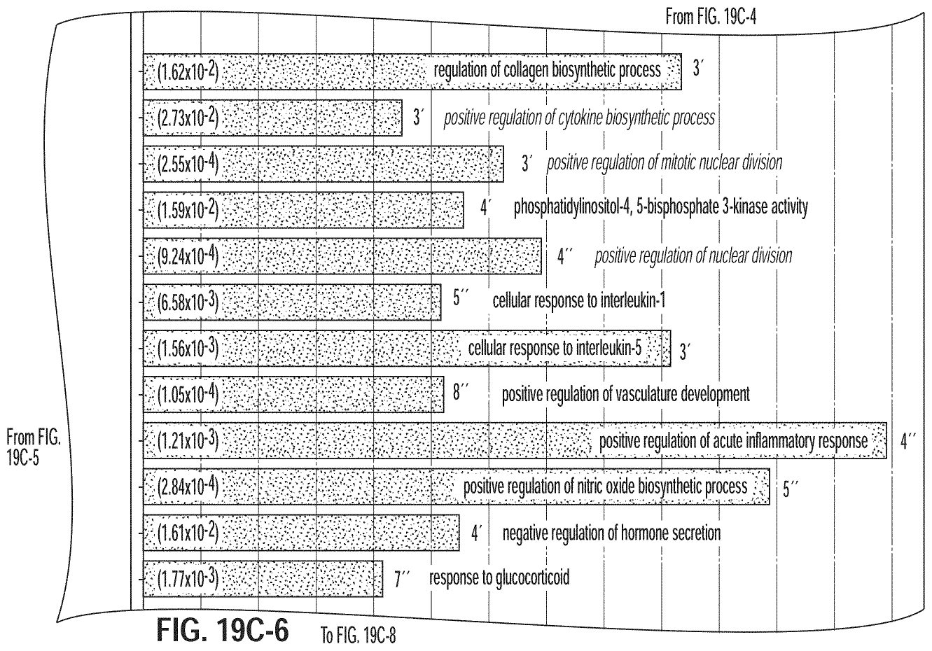

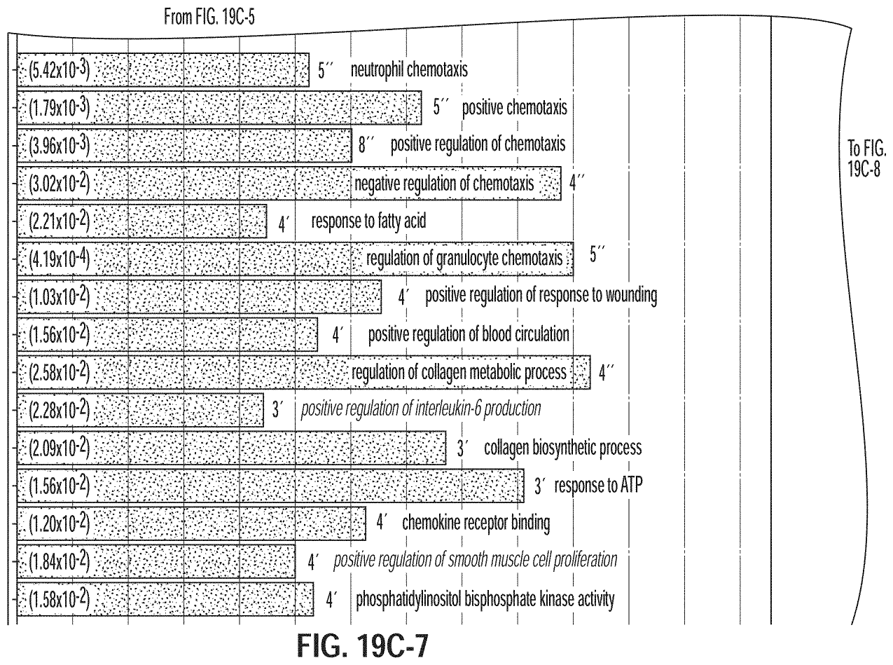

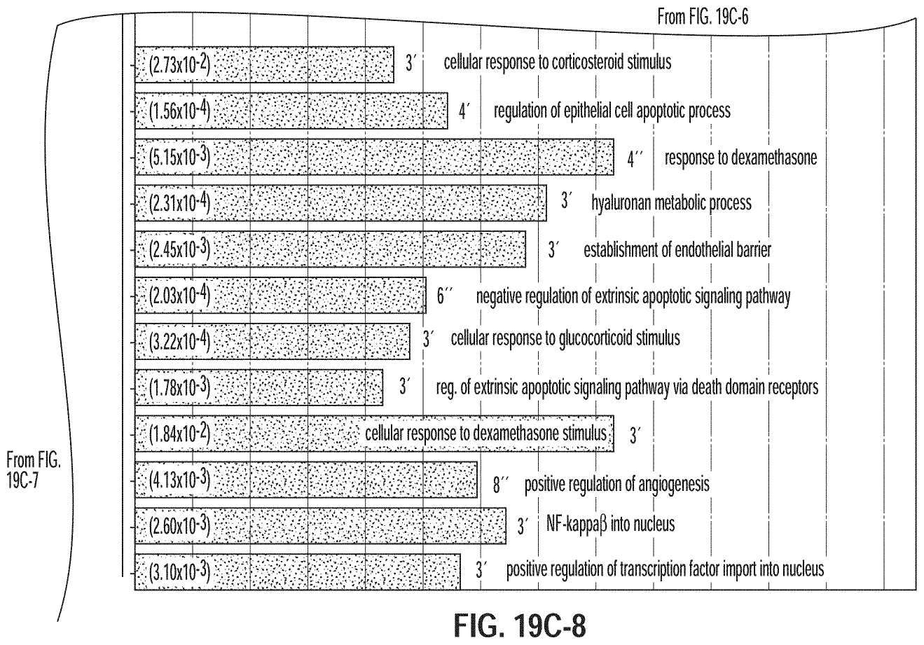

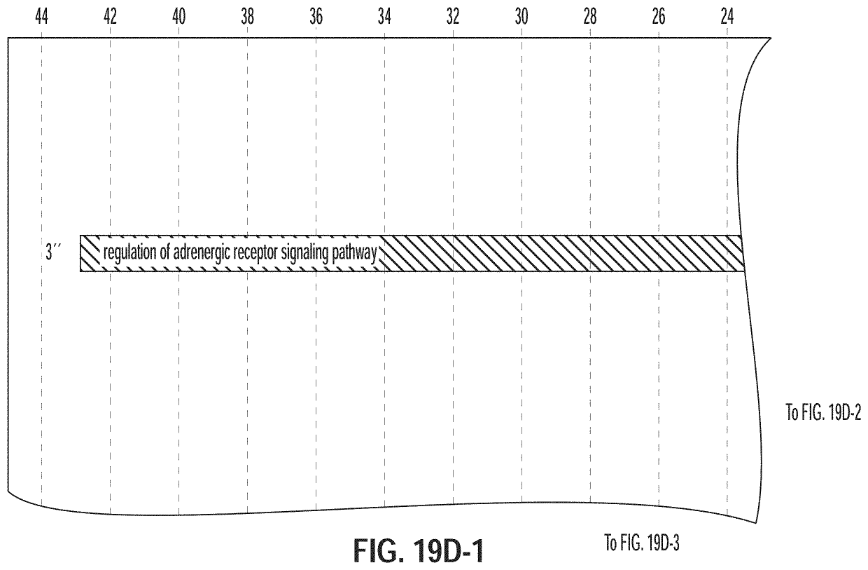

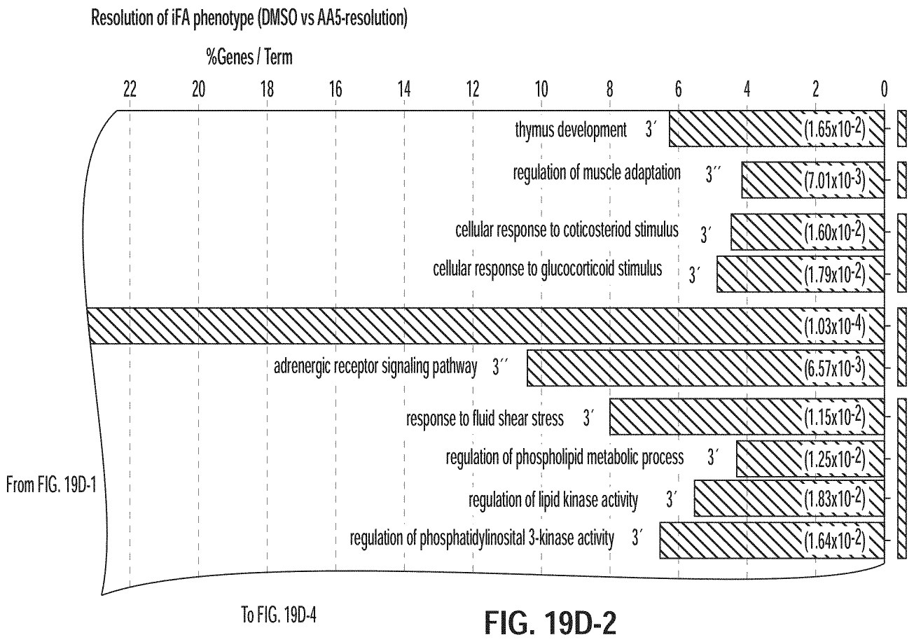

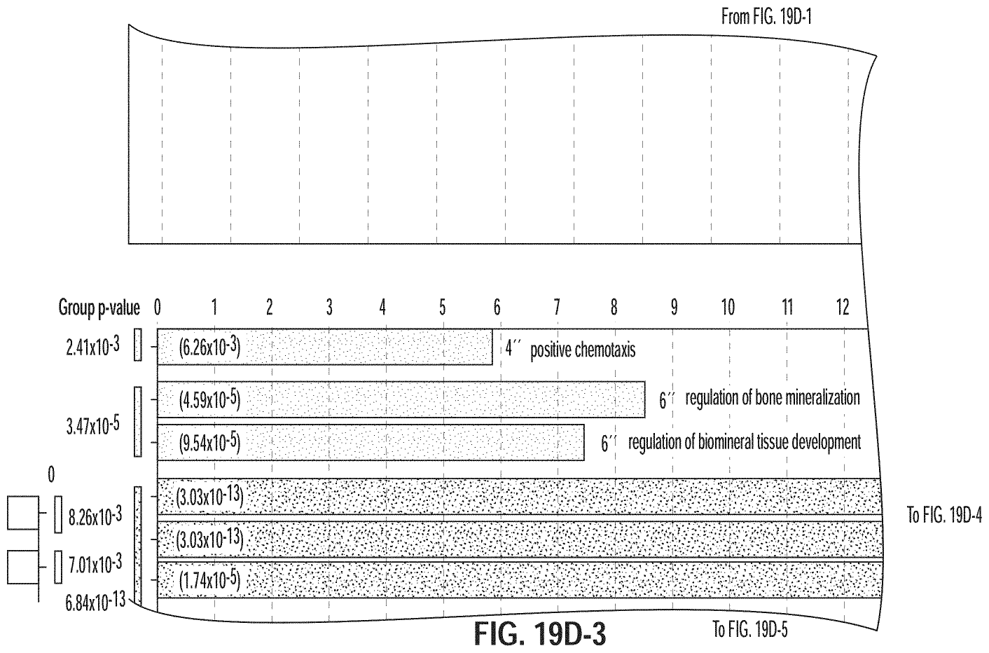

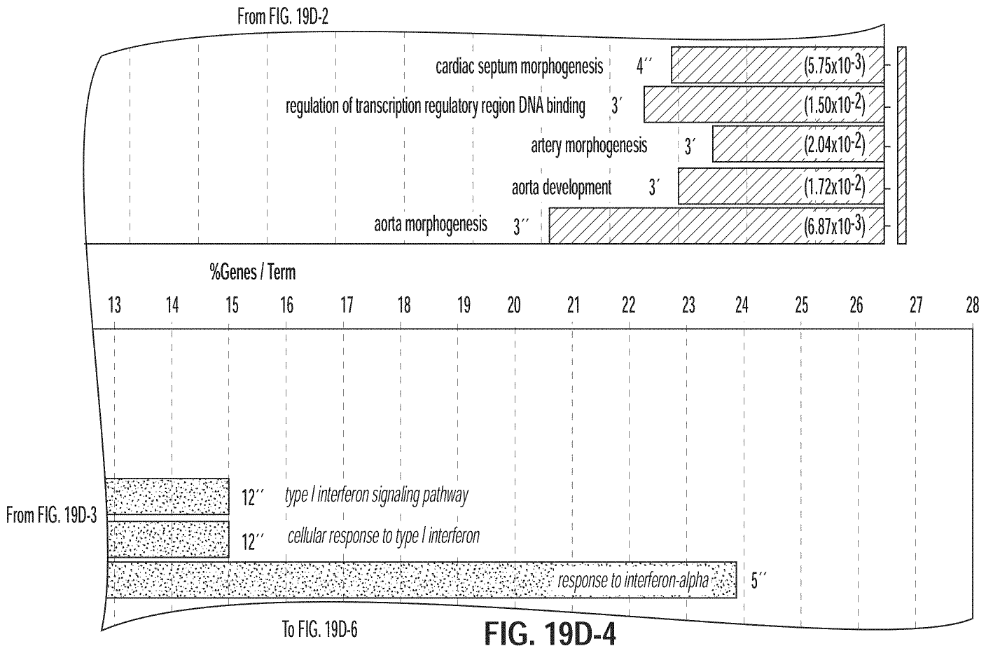

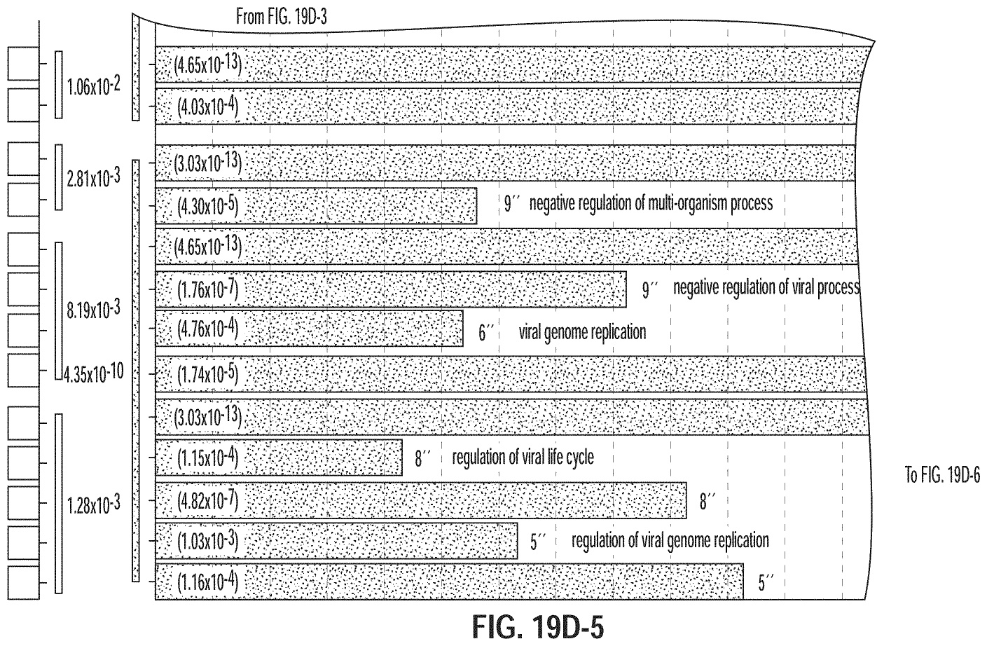

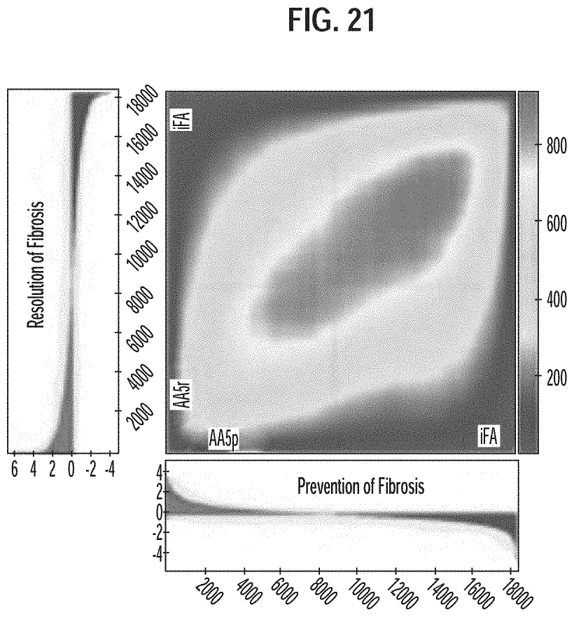

Transcriptomic analysis of the iFA model post-AA5-prevention and AA5-resolution treatments was performed using Rank Rank Hypergeometric Overlap (RRHO) analysis, which suggested overlapping mechanisms by which AA5 prevents and resolves the iFA phenotype (FIG. 21). RRHO analysis between the differentially expressed genes in healthy and IPF lung datasets compared to datasets of differentially expressed AA5-prevention (FIG. 19A) and AA5-resolution treated (FIG. 19B) genes. Medium to strong hypergeometric overlap was observed between treated (AA5-prevention or AA5-resolution) and untreated (fibrotic surrogate) iFA cultures as compared to the whole lung healthy and IPF tissues, suggesting AA5 treatment drives cells towards a healthier lung phenotype. These findings further indicate that the iFA model is a tractable model to study progressive fibrosis.

Differential gene expression analysis using RNA sequencing experiments in the iFA model treated with AA5 in both preventative (AA5-prevention) and resolutive (AA5-resolution) was performed. Functional cluster analysis using the ClueGo tool showed upregulation of gene ontology pathways such as response to acute-phase reactants, positive regulation of the acute inflammatory response and regulation of NF-κB import to the nucleus. Notably, positive regulation of chemotaxis and activation of acute-phase proteins such as IL-6, type I Interferon (IFN), and IL-1 were observed after both AA5-resolution and AA5-prevention treatments (FIGS. 18B,C and FIGS. 19C,D).

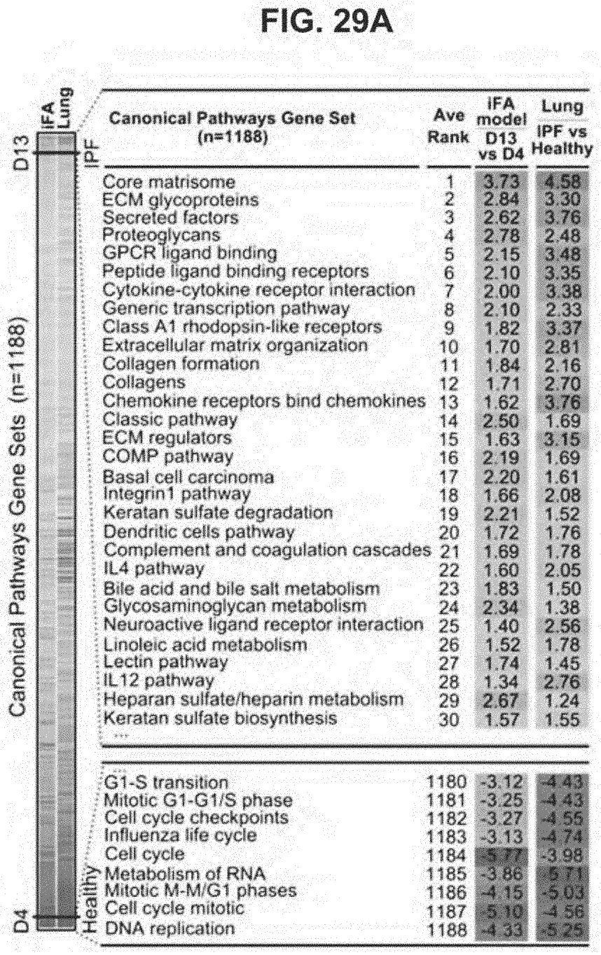

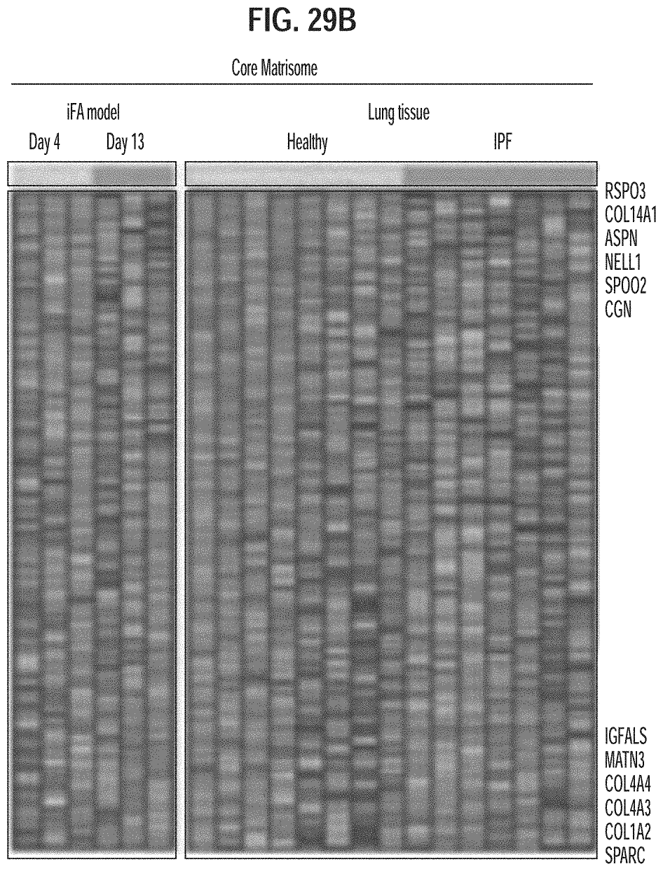

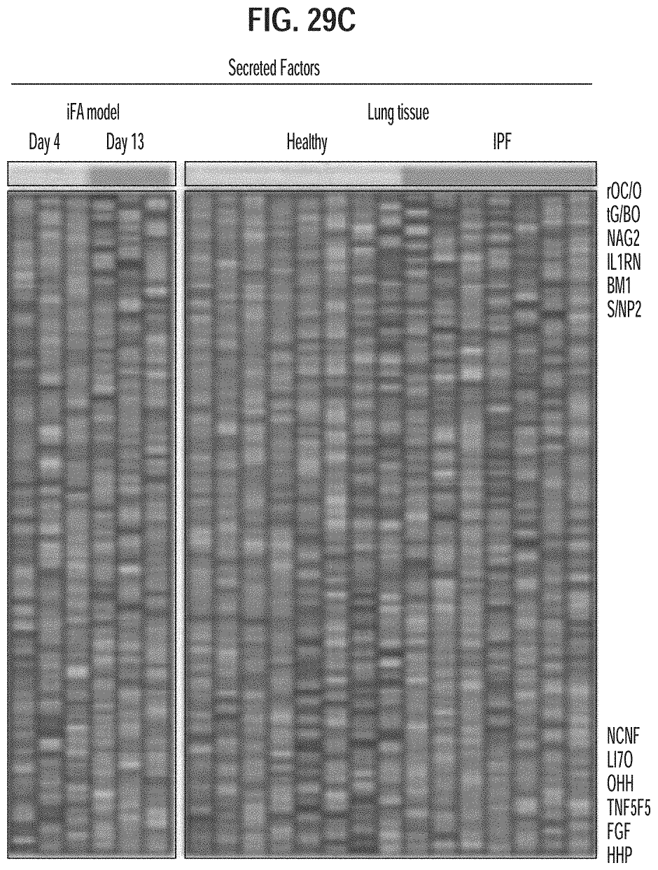

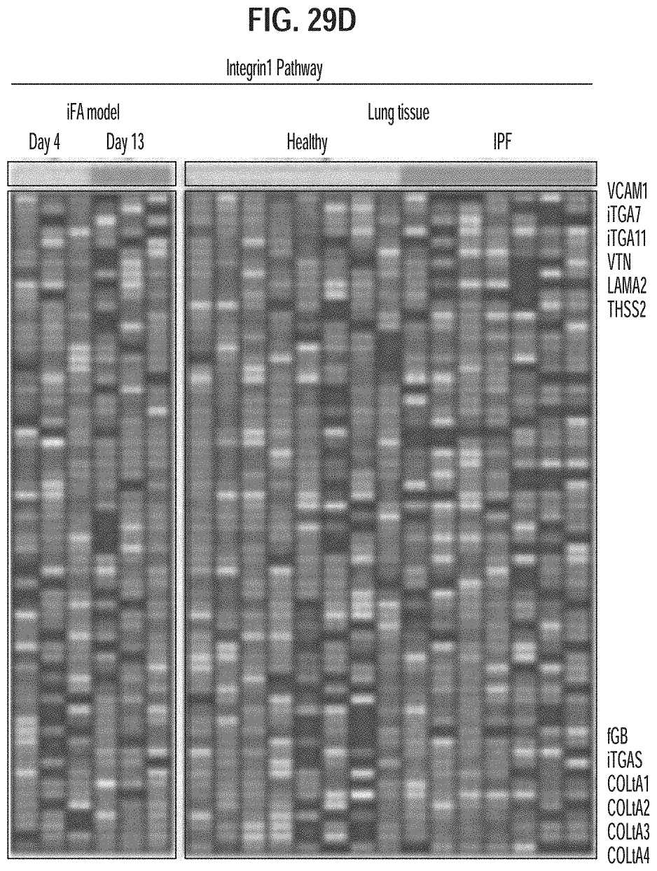

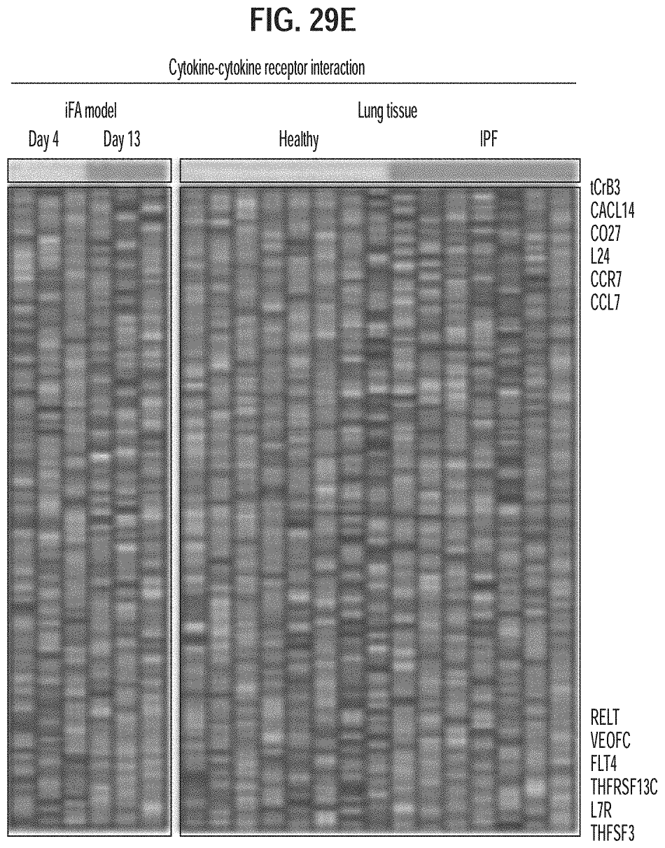

Gene Set Enrichment Analysis (GSEA) was performed to integrate the differential gene expression from RNA collected at day 4 (pre-iFA phenotype) and day 13 (established iFA phenotype) of the iFA model and to identify the top canonical pathways enriched during the progression of the iFA phenotype compared to a fibrotic lung using published dataset from healthy and IPF whole lung tissue (Nance et al., 2014) (FIG. 29A). The iFA model showed enrichment of genes encoding ECM including ECM glycoproteins, collagens, proteoglycans, and matrisome-associated secreted factors in a similar pattern to that seen in IPF lung tissue (FIG. 29B). Additionally, genes responsible for cell surface interactions (e.g. integrin 1 pathway) and regulation of inflammatory host defenses, cell growth and differentiation (e.g. cytokine-cytokine receptor interactions) were also enriched in both the iFA model and IPF lung tissues. On the other hand, healthy lung tissues and pre-iFA phenotype cultures shared similar expression of genes associated with normal cell function, including DNA replication and cell cycle pathways (FIGS. 29A-E). Taken together, this characterization of the iFA model indicated that it displays several features characteristic of inflammation-associated progressive fibrosis.

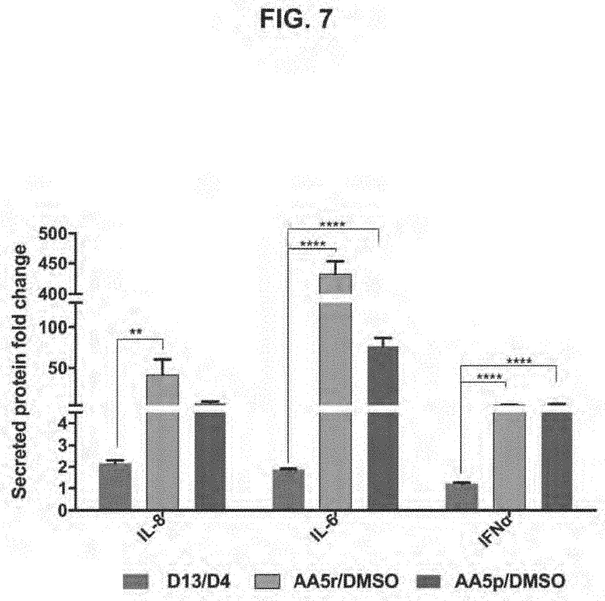

Because of the inflammatory signature induced by AA5-prevention and AA5-resolution, the acute-phase response in the iFA model treated with and without AA5 was examined. An increase in the secreted levels of the acute-phase response cytokines, IL-6, IL-8 and IFNα with both AA5-prevention and AA5-resolution treatments using luminex assays was observed (FIG. 7). Acute-phase response cytokines play a central role in regulating the innate immune response to infection, tissue injury, and DAMPs. Additionally, an increase was observed in the acute-phase signaling secondary cytokines, MCP-3, VEGF, CSF3 and GRO (FIG. 18F). Taken together, AA5 induced an acute-phase response and inhibition of HMGB1-mediated chronic cytokine signaling that is associated with scar resolution.

As a consequence of the acute-phase response, pentraxins (PTX) are released, and scavenging receptors such as CD163L1 on phagocytic cells are activated. Scavenger receptors such as CD163L1 play important roles in regulating tissue repair. The model showed upregulation of modulators of tissue repair, PTX3 and CD163L1, within 24 hours of treatment with AA5-resolution (FIG. 18D). The acute-phase signaling seen in the iFA model treated with AA5-prevention and AA5-resolution was consistently associated with progressively increasing amounts of PTX3 protein (FIG. 18E), which also correlated with the iFA prevention and resolution phenotypes (FIGS. 9,14,16,18). Taken together, AA5 appears to induce an acute-phase response that is associated with scar resolution.

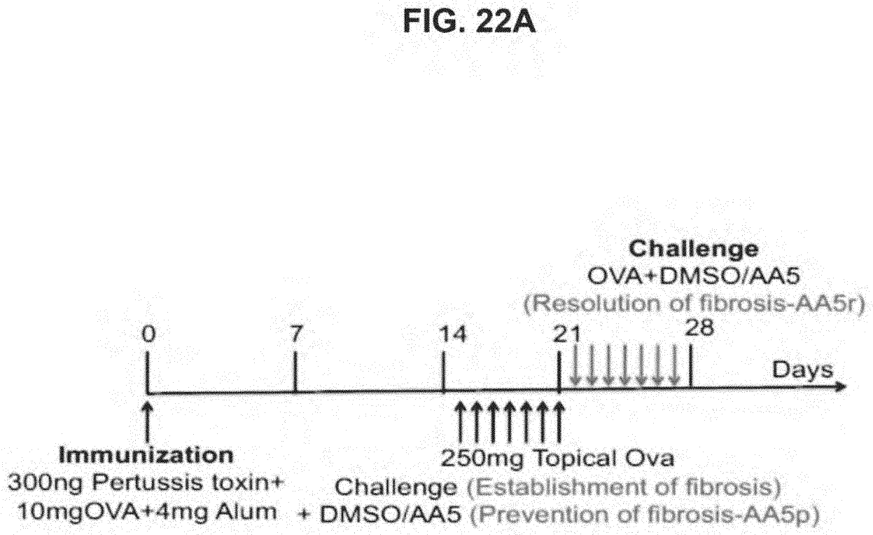

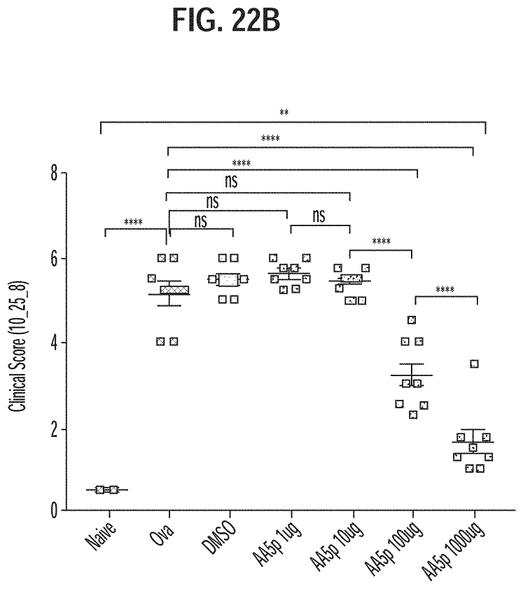

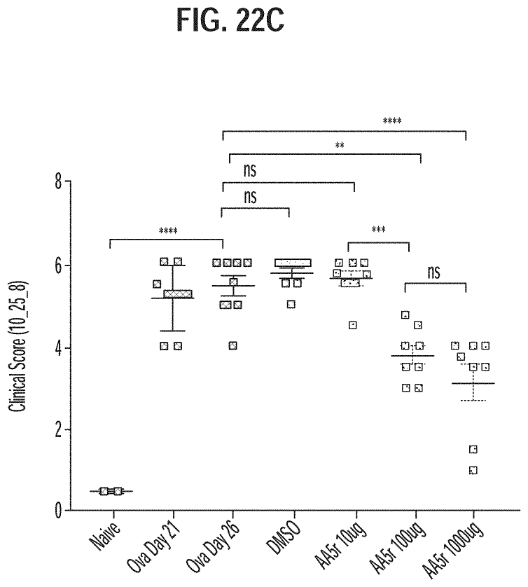

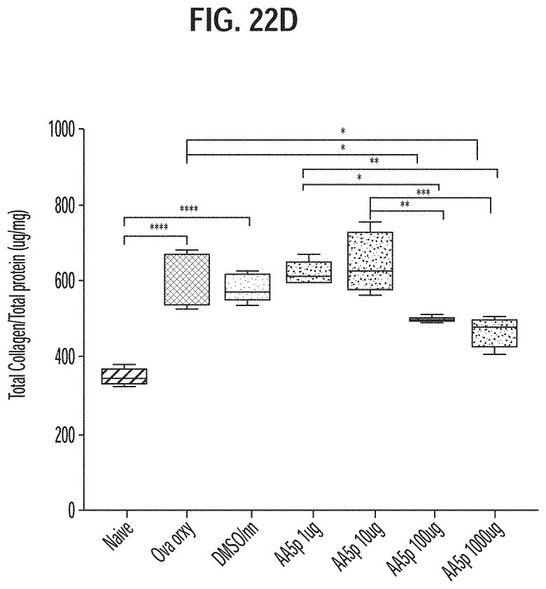

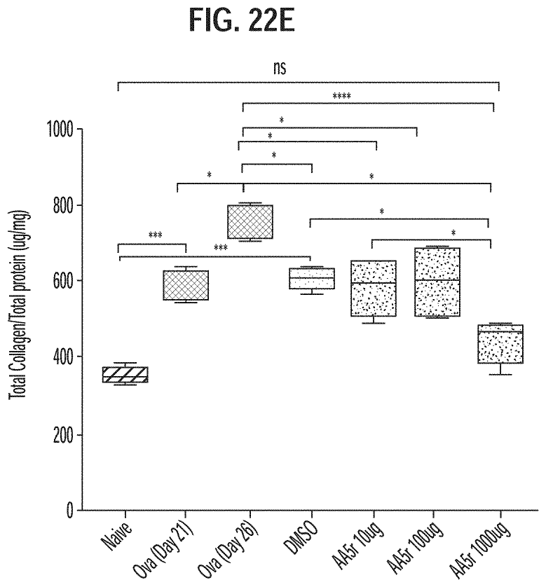

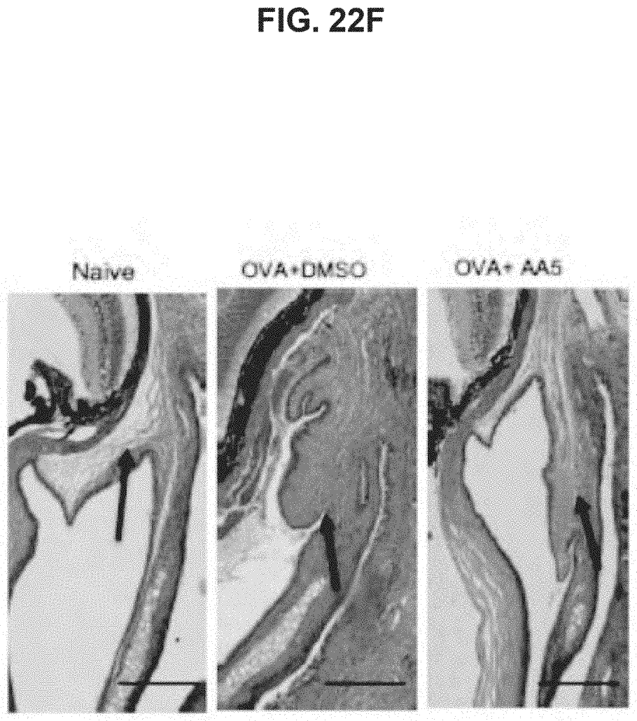

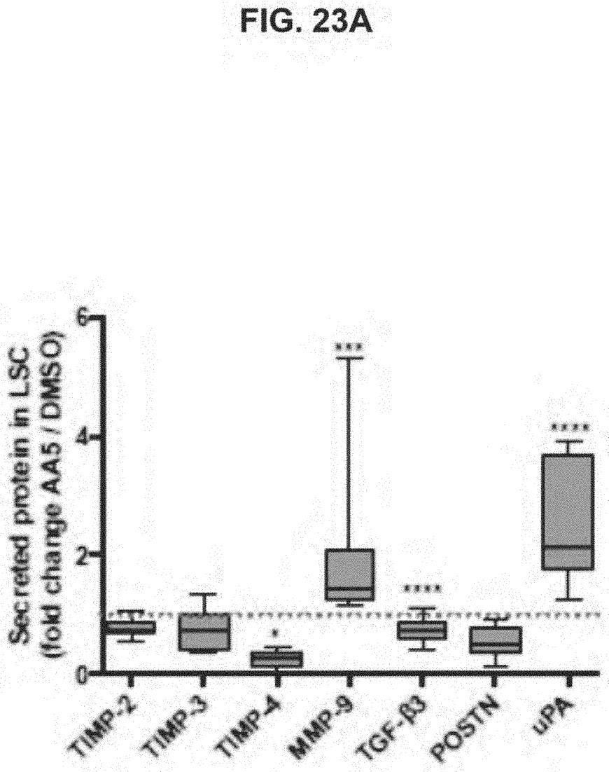

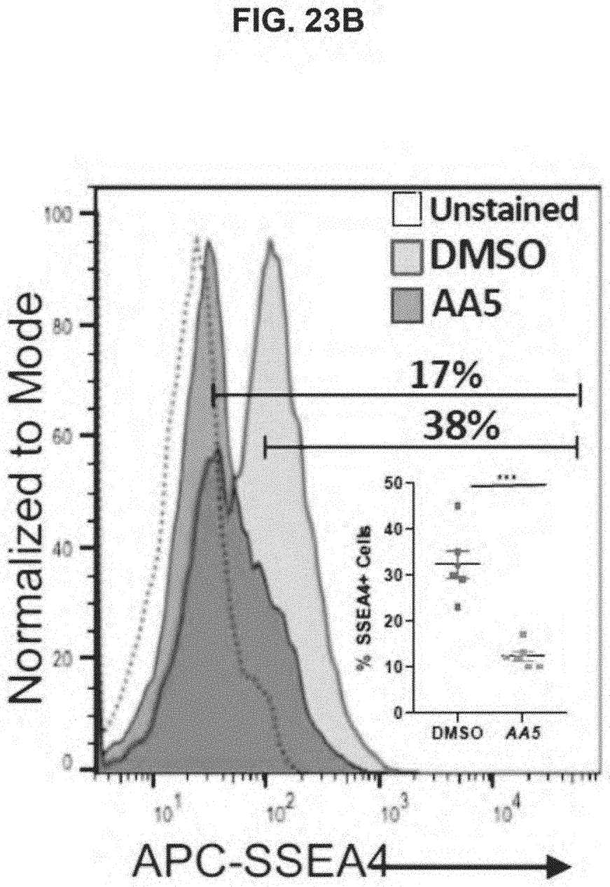

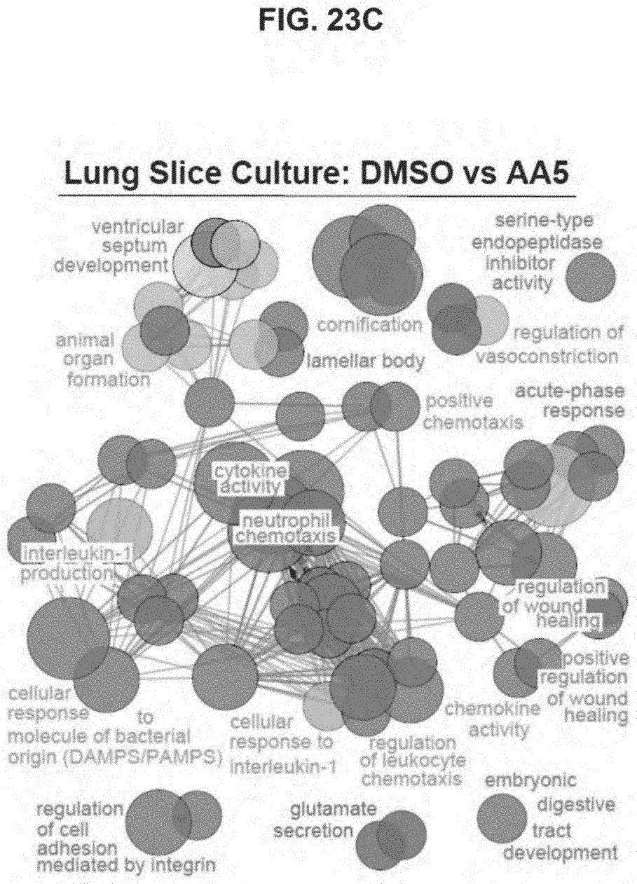

AA5 was further explored using an in vivo model of ocular mucosal fibrosis (FIG. 22) and an ex vivo model of lung fibrosis (FIG. 23). AA5 was evaluated in both prevention and resolution of fibrosis. As shown in (FIGS. 22A-C), AA5 treated eyes showed less clinical swelling, tearing and inflammation when compared to the DMSO-treated eyes in both the preventive and resolutive treatments. A significant reduction in the collagen content of the conjunctivae after AA5-prevention and AA5-resolution administration at 0.1 and 1 mg doses that correlated with the eye histology (FIGS. 22D-F) suggesting that AA5 was able to ameliorate OVA induced fibrosis in a dose dependent manner.

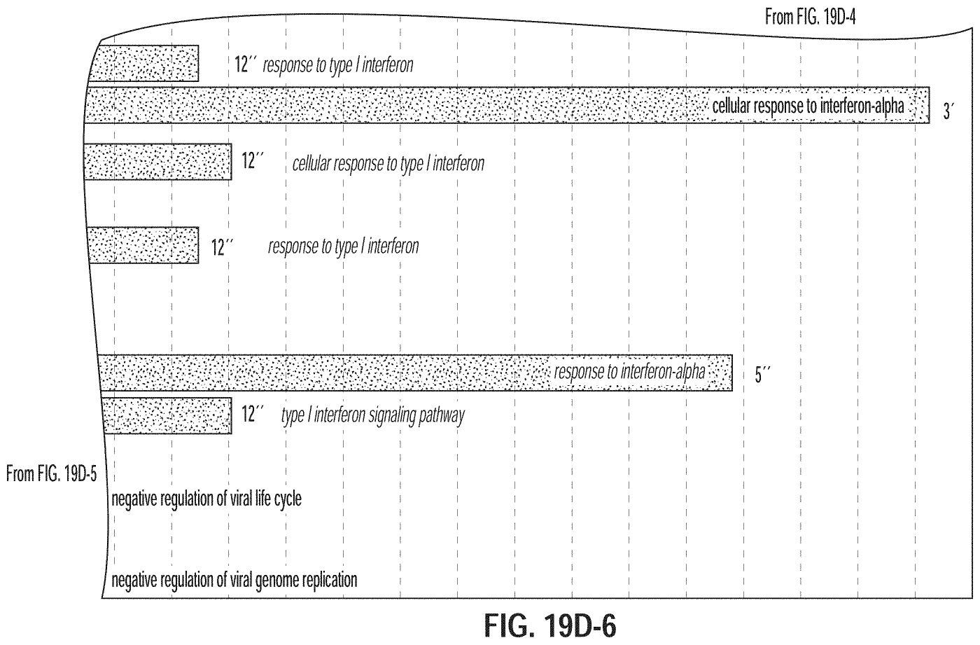

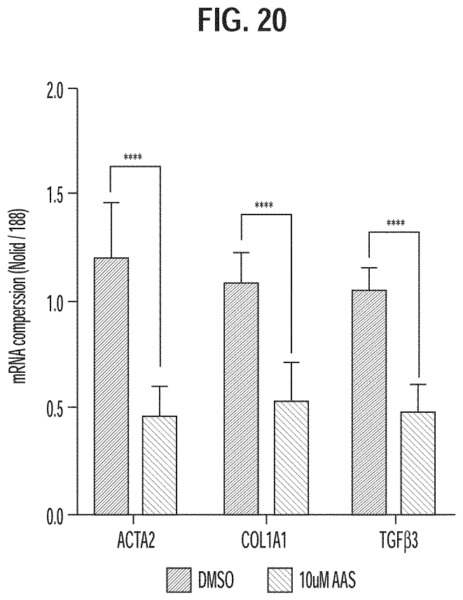

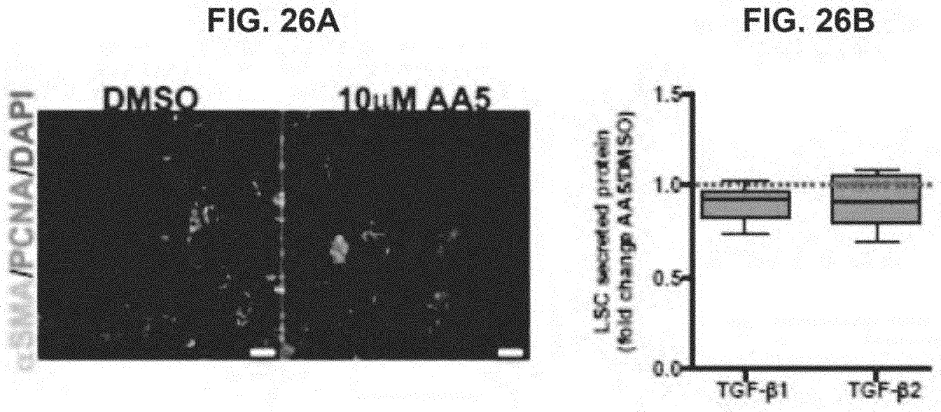

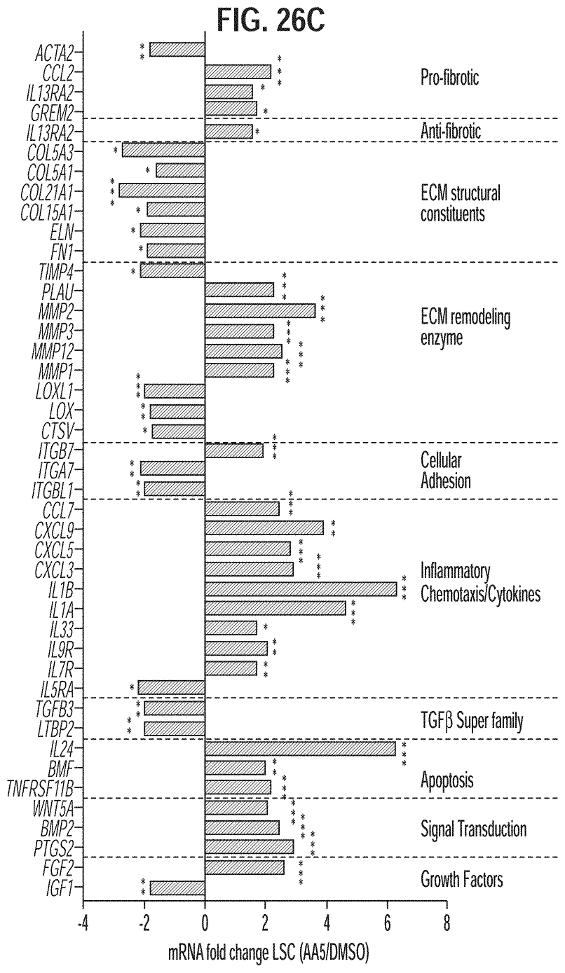

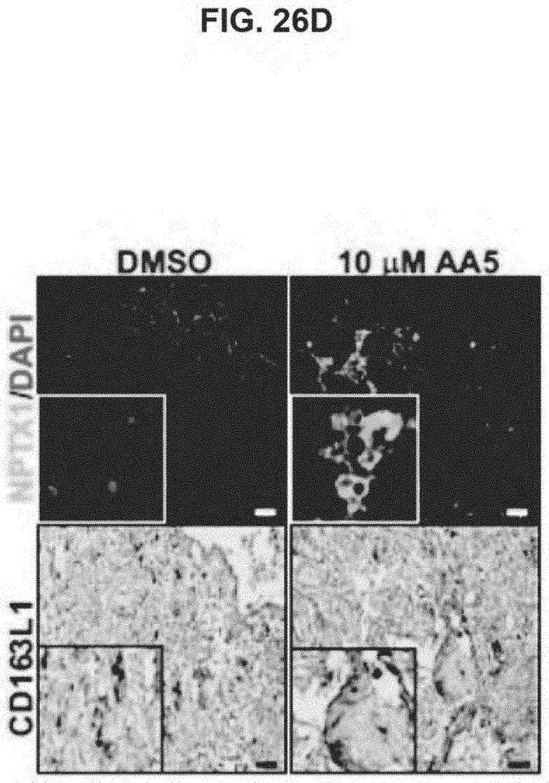

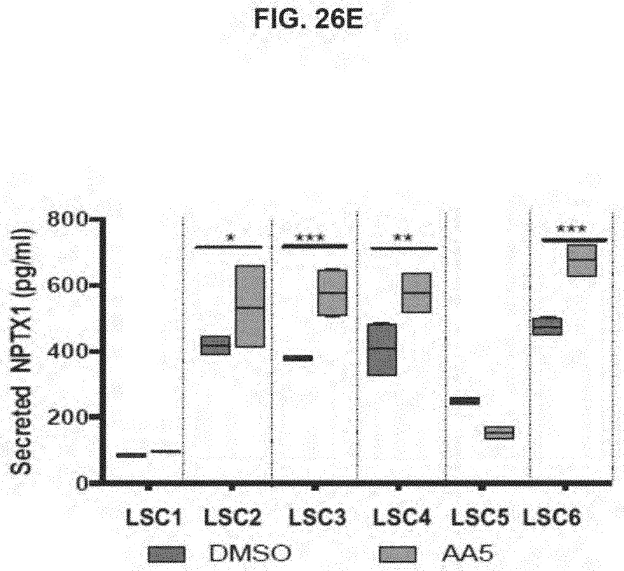

The efficacy of AA5 in a human ex vivo model using fibrotic lung samples was evaluated. A lung slice culture (LSC) system was established from end-stage Idiopathic Pulmonary Fibrosis patient lung tissue obtained at the time of lung transplantation. Thin fibrotic lung slices were incubated with AA5 or DMSO. The LSCs were viable at 48 hours as determined by immunostaining with proliferation marker, PCNA (FIG. 26A). Following culture, RNA was isolated and quantitative real-time PCR was used to determine expression of fibrosis markers. AA5 treatment significantly reduced ACTA-2, COL1A2, and TGF-β3 (but not TGF-β1/2) mRNA expression within 48 hours relative to DMSO-treated LSCs (FIG. 20). Further, decreased levels of secreted TGF-β3, (but not TGF-β1/2), TIMP-4, and POSTN protein expression and increased secretion of matrix degrading proteases such as MMP-9 and uPA with AA5 treatment were observed, consistent with a tissue repair response (FIG. 23A and FIGS. 26B,C). Immunostaining for HMGB1 revealed exclusively nuclear staining in the AA5-treated LSCs compared to the DMSO-treated LSC controls. This was further confirmed by the luminex assay that detected markedly decreased secreted HMGB1 with AA5 treatment (FIGS. 23B,C). Additionally, the fibrosis-initiating SSEA4+ cells were significantly reduced (>50%; p value <0.001) upon AA5 treatment (FIG. 27D).

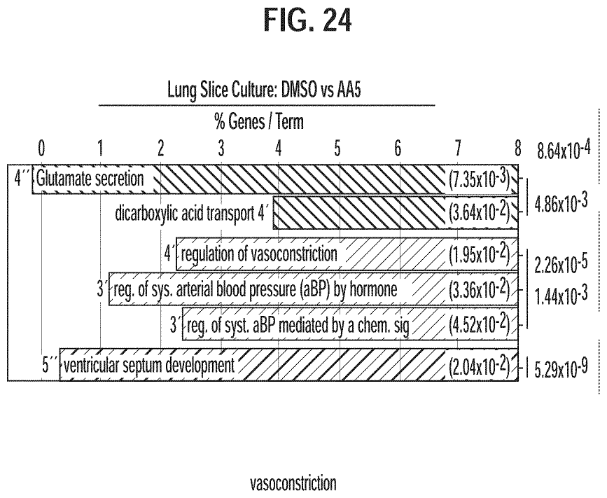

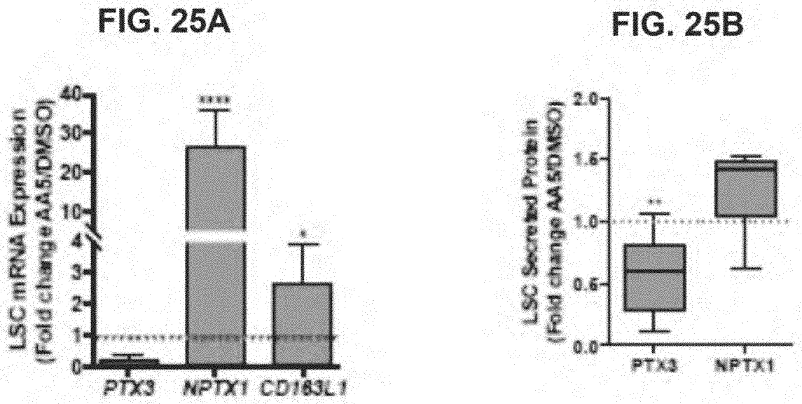

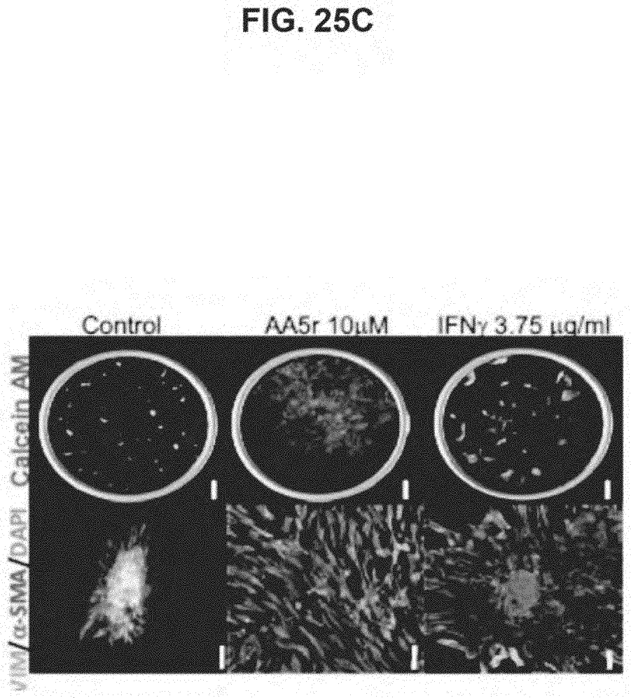

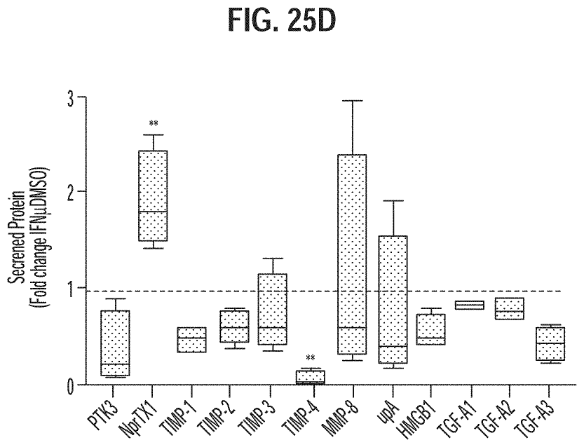

The cellular responses modulated by AA5 were evaluated using RNA sequencing on LSCs from 6 different IPF patients treated with AA5 or DMSO. Pathway enrichment analysis using ClueGo showed a significant enrichment of acute-phase signaling with nodes including neutrophil chemotaxis, IL-1 signaling, and cellular response to DAMPs (FIG. 24). Therefore, as with the iFA model, activation of acute-phase response genes and fibrosis resolution genes with AA5 treatment were consistently observed in the LSCs. Instead of increasing levels of secreted protein PTX3 in LSCs, the paralog of PTX3, neuronal pentraxin 1 (NPTX1) was observed to be upregulated in the LSCs at the mRNA and protein level (FIGS. 25A,B and FIGS. 26D,E). Also, the scavenger receptor CD163L1 was upregulated and secreted in the AA5-treated LSCs (FIGS. 25A and 26D).

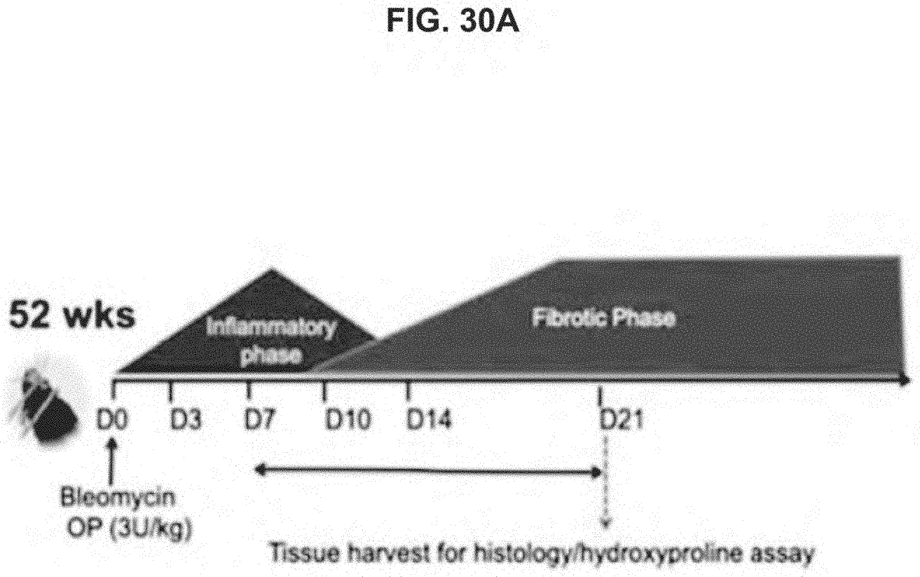

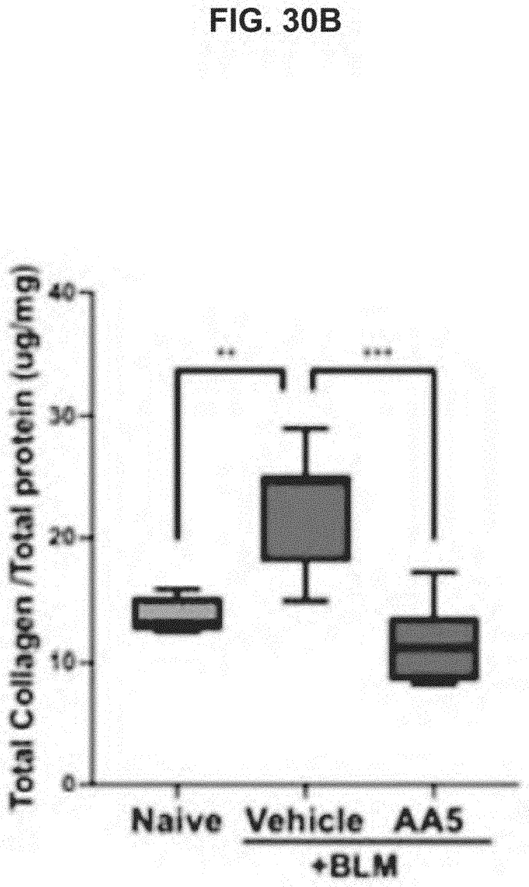

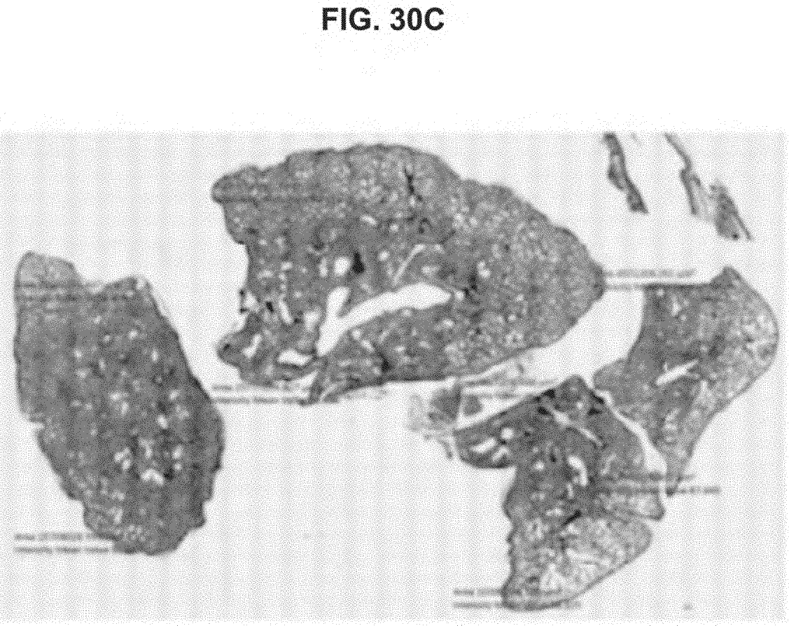

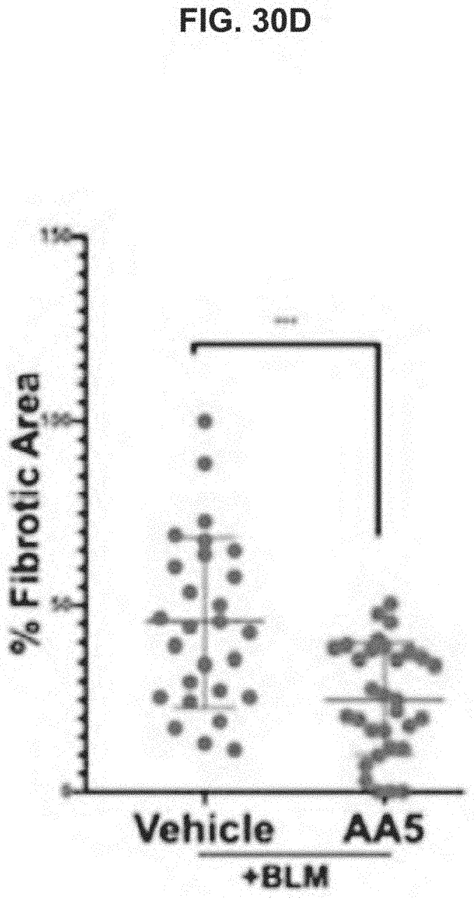

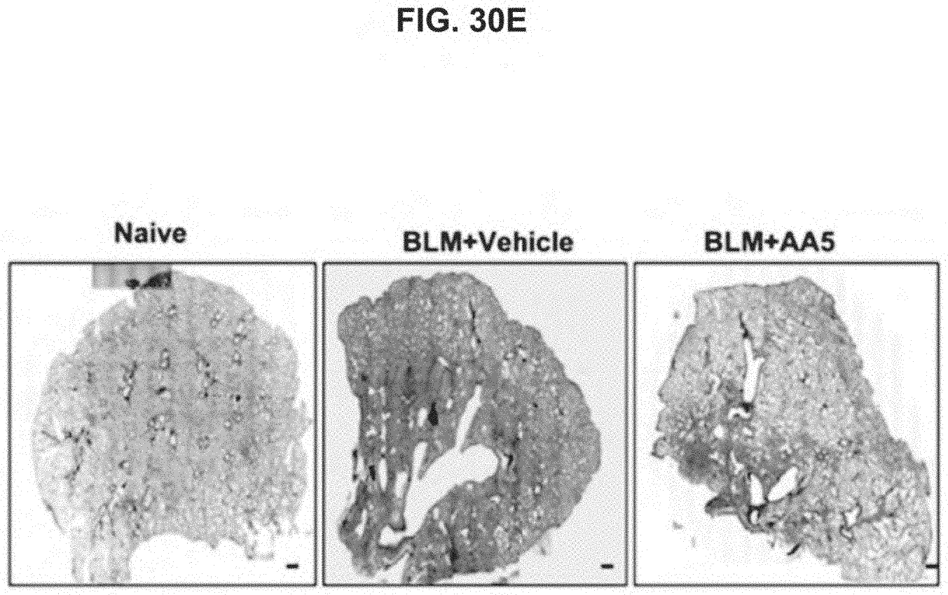

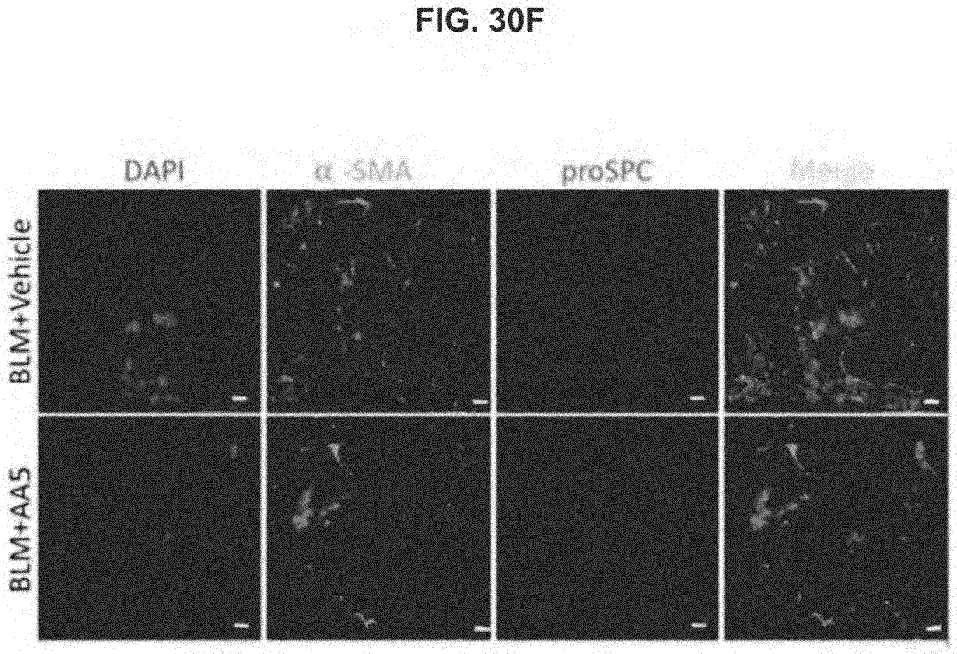

To define the anti-fibrotic effect of AA5, a bleomycin model of lung injury in aged mice was used as a model of lung fibrosis (FIG. 30A). 52-week-old mice were challenged with bleomycin (3 U/kg) by the oropharyngeal route to induce lung injury, followed by systemic administration of AA5 (20 mg/kg) or DMSO, from days 7-21 post bleomycin injury. Naïve mice were maintained as controls. The mice were sacrificed on day 21 and the total collagen content of the lungs was quantified using the hydroxyproline assay. Bleomycin challenged mice had elevated amounts of total collagen compared to their naïve controls In contrast, mice treated with AA5 demonstrated significantly reduced total collagen compared to the vehicle treated controls (FIG. 30B). The percentage of fibrotic areas from each section was quantified using hematoxylin and eosin staining, tiled and imaged on a Zeiss Axioscope microscope at 2.5× and quantified using the spline contour tool using the ZEN 2011 software. This demonstrated that the vehicle treated mice displayed significantly more fibrotic areas compared to the AA5 treated animals following challenge with bleomycin (FIGS. 30C-E). A qualitatively larger number of the type II marker pro-Surfactant Protein C (proSPC) was observed in bleomycin challenged mice treated with AA5 suggesting epithelial regeneration compared to the vehicle treated controls (FIG. 30F). Taken together, AA5 displays significant anti-fibrotic activity as seen from in vivo models of ocular mucosal fibrosis and pulmonary fibrosis.

IFNs have immune-regulatory effects and are known anti-fibrotic cytokines. To further confirm the activity of AA5, the iFA model was treated with recombinant IFNγ and this partially rescued the iFA phenotype (FIG. 25C). Consistent with this, treatment of the LSCs with recombinant IFNγ also revealed a similar expression profile of fibrosis related resolution proteins when treated with AA5 (FIG. 25D). This was accompanied by regulation of complex fibro-suppressive inflammatory processes such as activation of MMPs and uPA, downregulation of TIMPs, elastin, fibronectin, collagens, LOX, and secreted DAMPs. Thus, AA5 exhibits its anti-fibrotic effect by activating an acute-phase response with cytokines such as IL-6 and IFNs. The data suggest that AA5 exhibits its anti-fibrotic activity by activating an acute-phase response and playing an immune regulatory role.

The disclosed model phenocopies all of the fibrogenic response phases that include initiation of the injury, activation of effector cells followed by production of ECM and then failure to resorb the ECM with continued deposition of ECM. The model mimics all four fibrotic phases with the secretion of the DAMP, HMGB1, the activation of fibroblasts to myofibroblasts, activation of TGFβ, the upregulation of inflammatory cytokines and chemokines and the progressive deposition of collagen, thereby creating a phenotypic surrogate for progressive fibrosis.

Known models for fibrosis utilize the exogenous addition of pro-fibrotic modulators such as TGF-β to their cultures to drive a fibrotic response for 48 or 72 hours. At the end of the treatment period, gene and protein expression for markers of myofibroblast activation and ECM production are investigated. This approach uses a time course that is may be too short to model disease progression and is also subject to modulator-driven bias. In contrast, the disclosed model does not utilize the addition of any external fibrotic modulators, so it is unbiased and target-agnostic for progressive fibrosis disease modeling and drug discovery. Further, other current in vitro models only evaluate fibrosis inhibition, so unlike the present model, these models cannot be used to assess a reversal of fibrosis.

The present model using iPSCs is able to generate its phenotype because these cells are capable of differentiating to multiple different cell types and each cell type is plastic and can give rise to the other cell types by epithelial-mesenchymal-transition or mesenchymal-epithelial-transition. Fibrosis is a very plastic process with a dynamic interplay between ECM deposition and regression as well as plasticity in effector cell populations. In addition, the model is able to alter the inflammatory milieu and switch the fibrotic process to a resolutive one which enables studies of this phase of fibrosis, which some known models lack. For example, the model recapitulates the activation of NF-κB and TGF-β which induces fibroblast/myofibroblast activation and ECM deposition and AA5 appears to restore the balance by activation of pentraxins and macrophage scavenger receptor upregulation. The model represents both the chronic and acute inflammation of injury and repair in vitro and enabled determination that AA5 inhibits and reverses fibrosis by activating acute inflammatory signals. These data suggest that approaching inflammation-driven fibrosis by targeting endogenous agonists of resolution may offer an attractive strategy to treat idiopathic pulmonary fibrosis.

### Pharmaceutical Compositions

In certain embodiments, the present invention provides a pharmaceutical preparation suitable for use in a human patient, comprising any of the agents shown above, and one or more pharmaceutically acceptable excipients. In certain embodiments, the pharmaceutical preparations may be for use in treating or preventing a condition or disease as described herein. Any of the disclosed agents may be used in the manufacture of medicaments for the treatment of any diseases or conditions disclosed herein.

The compositions and methods of the present invention may be utilized to treat a subject in need thereof. In certain embodiments, the subject is a mammal such as a human, or a non-human mammal. When administered to subject, such as a human, the composition or the agent is preferably administered as a pharmaceutical composition comprising, for example, an agent of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of an agent such as agent of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, an agent of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those agents, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The agent may also be formulated for inhalation. In certain embodiments, an agent may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the agent which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active agent, such as an agent of the invention, with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent of the present invention as an active ingredient. Compositions or agents may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered agent moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active agent may be mixed under sterile conditions with a pharmaceutically acceptable carrier, and with any preservatives, buffers, or propellants that may be required.

The ointments, pastes, creams and gels may contain, in addition to an active agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active agent, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a agent of the present invention to the body. Such dosage forms can be made by dissolving or dispersing the active agent in the proper medium. Absorption enhancers can also be used to increase the flux of the agent across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active agents can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of an agent at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular agent, or the ester, salt or amide thereof, the route of administration, the time of administration, the rate of excretion of the particular agent being employed, the duration of the treatment, other drugs, compounds and/or materials used in combination with the particular agent employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or agent at levels lower than that required in order to achieve the desired therapeutic effect and gradually increase the dosage until the desired effect is achieved. By “therapeutically effective amount” is meant the concentration of an agent that is sufficient to elicit the desired therapeutic effect. It is generally understood that the effective amount of the agent will vary according to the weight, sex, age, and medical history of the subject. Other factors which influence the effective amount may include, but are not limited to, the severity of the subject's condition, the disorder being treated, the stability of the agent, and, if desired, another type of therapeutic agent being administered with the agent of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active agent used in the compositions and methods of the invention will be that amount of the agent that is the lowest dose effective to produce a therapeutic effect. Such an effective dose will generally depend upon the factors described above.

If desired, the effective daily dose of the active agent may be administered as one, two, three, four, five, six or more sub-doses administered separately at appropriate intervals throughout the day, optionally, in unit dosage forms. In certain embodiments of the present invention, the active agent may be administered two or three times daily. In preferred embodiments, the active agent will be administered once daily.

In certain embodiments, agents of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of agents of the invention with one or more additional therapeutic agent(s) (e.g., one or more additional therapeutic agent(s)) provides improved efficacy relative to each individual administration of the agent of the invention or the one or more additional therapeutic agent(s). In certain such embodiments, the conjoint administration provides an additive effect, wherein an additive effect refers to the sum of each of the effects of individual administration of the agent of the invention and the one or more additional therapeutic agent(s).

This invention includes the use of pharmaceutically acceptable salts of agents of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Exemplification

The invention now being generally described, it will be more readily understood by reference to the following examples, which are included merely for purposes of illustration of certain aspects and embodiments of the present invention, and are not intended to limit the invention.

### Generation of Induced Pluripotent Stem Cells

For the generation of iPSCs, skin and lung biopsies and PBMCs were procured with appropriate patient consent and institute IRB approval. The iPSCs were generated according to established protocols (Karumbayaram et al., 2012 Stem cells translational medicine 1, 36-43). Briefly, the punch biopsy samples were chopped and incubated in 2% animal origin free collagenase solution for 90 min at 37° C. The dissociated cells were plated in MSCGM-CD (Lonza) medium to generate primary fibroblasts. For the generation of iPSC's, 1×10⁵ primary fibroblasts were transduced with STEMCAA (gift from Dr. Darrell Kotton, Boston University, MA) vector concentrate (7×10⁶ TU/ml) in MSCGM-CD medium. Five days post-infection, cells were re-plated in 50:50 TeSR2/Nutristem containing 10 ng/ml bFGF until iPSC-like colonies appeared. The colonies were picked mechanically and cultured in CELLstart-coated dishes. Three independent iPSC lines per tissue sample were generated.

FIG. 1A shows the first steps in the generation of mesenchymal-like cells such as Primary cultures of lung fibroblasts that expressed the fibroblast marker FSP1. FIG. 2B shows the primary fibroblasts were reprogrammed into iPSC and expressed pluripotency marker SOX2.

Differentiation of iPSC into Mesenchymal-Like Cells to Model Progressive Fibrosis.

Differentiation of iPSC for modeling progressive fibrosis was generated according to published protocols (Wilkinson et al., 2016, Stem cells translational medicine). Briefly, iPSCs were dissociated using 1 mg/ml of dispase, rinsed twice and then cultured in non-adherent dishes in DMEM/F12 medium supplemented with 10% FBS, 1× Glutamax, 10 nM Non-essential amino acids and 0.1 mM monothioglycerol (MTG) for the generation of embryoid bodies. After 4 days, the embryoid bodies were collected gently and plated on gelatinized dishes to allow to adhere and cultured in media containing DMEM/F12 medium supplemented with 10% FBS, 1× Glutamax and 10 nM non-essential amino acids and allowed to differentiate for an additional two weeks. The outgrowths from the embryoid bodies were collected by trypsinization, and passaged for expansion and cryopreservation.

FIG. 1C shows the iPSCs were differentiated into cells that expressed markers that were largely mesenchymal-like cells (Vimentin, VIM). FIG. 1D shows an overlaid histogram plot that depicts the relative SSEA4 fluorescence intensity (blue) of the mesenchymal-like cells compared to the unstained controls (grey). Inset depicts % of positive cells (n=17). p≤0.0001 by Wilcoxon signed rank test. FIG. 1E shows an overlaid histogram plot that depicts the relative CD326 (epithelial marker) fluorescence intensity (blue) of the mesenchymal-like cells compared to the unstained controls (grey).

FIG. 1F shows representative FACS plots revealing expression of monocyte/macrophage markers in the mesenchymal-like cells. The cells were co-stained for CD68 and CD32, CD11b or CD14, revealing 0.2-0.7% cells positive for monocyte/macrophage markers. Unstained controls are shown in top panel (n=7). p=0.0156 by Wilcoxon signed rank test. FIG. 1G shows a representative image of mesenchymal-like cell of a macrophage-like cell displaying heterochromatin (+), vacuolated cytoplasm (*), several microvilli (arrows) and whorls of phagocytosed matter (star). Scale bar, 1 μm. FIG. 1H shows characterization of primary fibroblasts (middle panel) and mesenchymal-like cells (right panel) using multi-color FACS. Unstained control plots are shown on the left panel. Representative FACS data with gating are shown for each marker.

Generation of an Induced Fibroblast Activation (iFA) Phenotypic Surrogate of Progressive Fibrosis

Mesenchymal-like cells were cultured on 13 kPa functionalized polyacrylamide gels that were prepared as follows. 187.5 μl of 40% acrylamide, 60 μl of 2% bis-acrylamide and 8.55 μl of sodium bisulfate in a final volume of 990 μl of water was incubated for 20 min at room temperature to degas the mixture. To this mixture, 0.10% ammonium persulfate and 0.15% TEMED were added, mixed and 100 μl of the solution was added onto a 0.4% 3-(Trimethoxysily) propyl methacrylate pH 3.5 treated coverslip. A 2% dimethyl dichlorosilane in chloroform treated round glass coverslip was then inverted on the acrylamide solution and allowed to polymerize for 15 min between the two surfaces. The top coverslip was gently removed, and the bottom coverslips with the hydrogel were transferred to appropriate multi-well low adherent tissue culture plate containing coupling buffer (0.1 M sodium phosphate dibasic, 0.1 M sodium phosphate monobasic) pH 8. To functionalize the hydrogel, 1 part of 0.1 M p-benzoquinone in dioxane was added to the hydrogels containing 4 parts of coupling buffer and incubated at 37° C. for 30 min. The gel was then successively washed with 20% dioxane in water, water, 0.1 M sodium acetate buffer at pH 4.0 containing 1.0 M sodium chloride, 0.1 M sodium bicarbonate solution at pH 8.5 containing 1.0 M sodium chloride, and finally with coupling buffer at pH 7.5. The hydrogels were then coated with 0.1% gelatin for 2 hrs prior to seeding the cells. Cells were seeded at a density of 3000 cells/cm².

Comparative IF images of primary fibroblasts and mesenchymal-like cell cultures stained for fibroblast marker (FSP-1) and mesenchymal marker (VIM) are shown in FIG. 2A. The mesenchymal-like cells and primary fibroblasts were cultured on 13 kPa hydrogels. Phase contrast images show monolayer cultures of the primary fibroblasts grown on hydrogels while the mesenchymal-like cells heaped up as aggregates (also called induced fibroblast activation (iFA) cultures) as shown on day (D) 13. FIG. 2B provides comparative IF images of primary fibroblasts and mesenchymal-like cell cultures on days 1, 3, 6 and 9 stained with α-SMA, Vimentin (VIM) and EdU after a 6-hour EdU treatment.

FIG. 2C shows that representative IF images of decellularized primary fibroblast (control) and iFA cultures stained for Collagen I revealed disorganized ECM in the iFA cultures (top right panel) compared to the organized ECM in primary fibroblast cultures (top left panel). Bottom right panel shows iFA cultures expressed α-SMA, qualitatively more than primary fibroblasts (bottom left panel). FIG. 2D shows single positive cells for SSEA4, CD105 and CD326 (negative for CD45) were sorted from the iFA model and re-cultured on 13 kPa hydrogels. Within 13 days, the single positive populations were all able to reproducibly generate the iFA phenotype (n=3).

FIG. 2E shows the iFA model created from single positive cells of CD105, SSEA4 and CD326 in FIG. 2A were individually reanalyzed using FACS, revealing that each of the single positive populations were able to give rise to the other populations suggesting that the mesenchymal-like cells are plastic. Unstained controls plots are shown on the left panel. Representative FACS data with gating are shown for each marker.

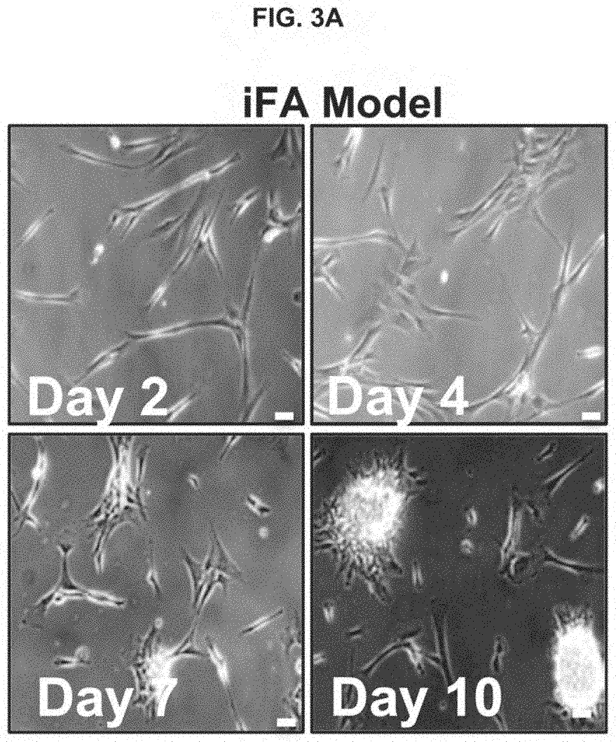

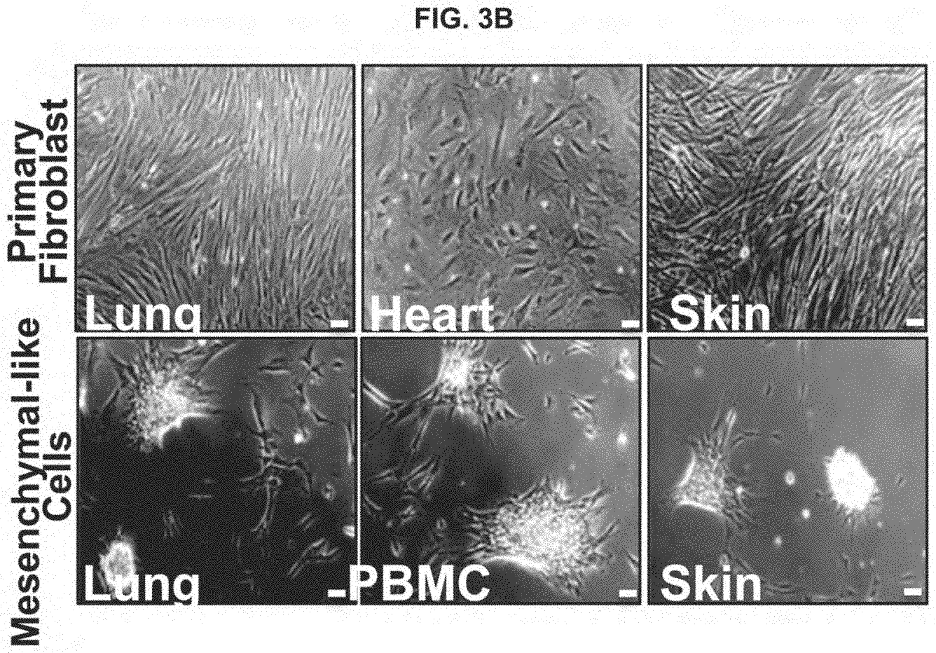

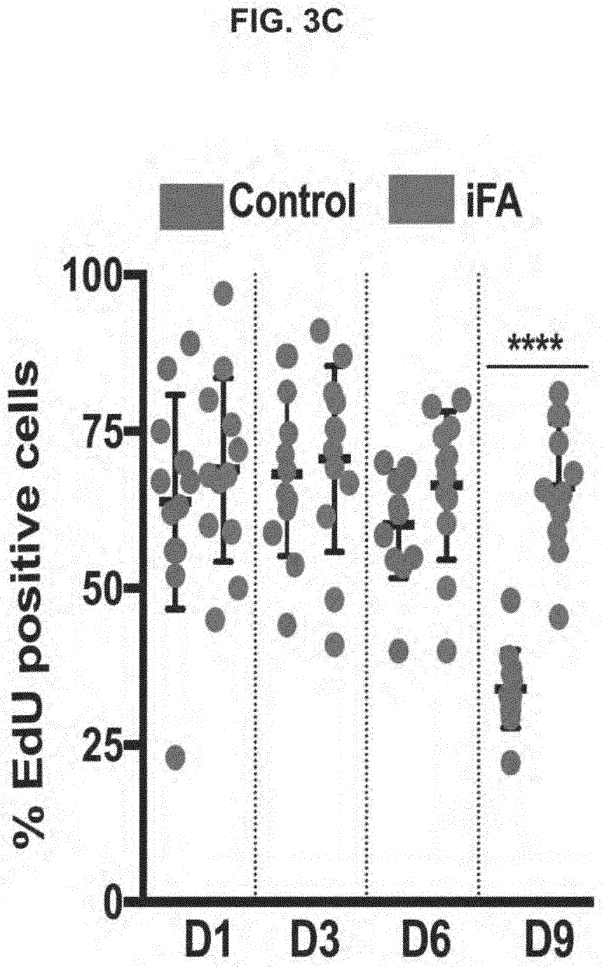

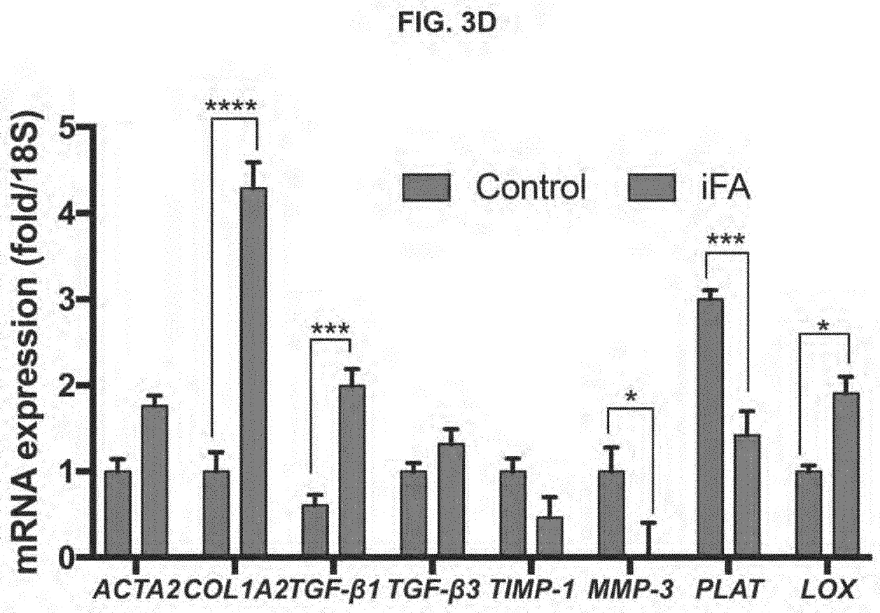

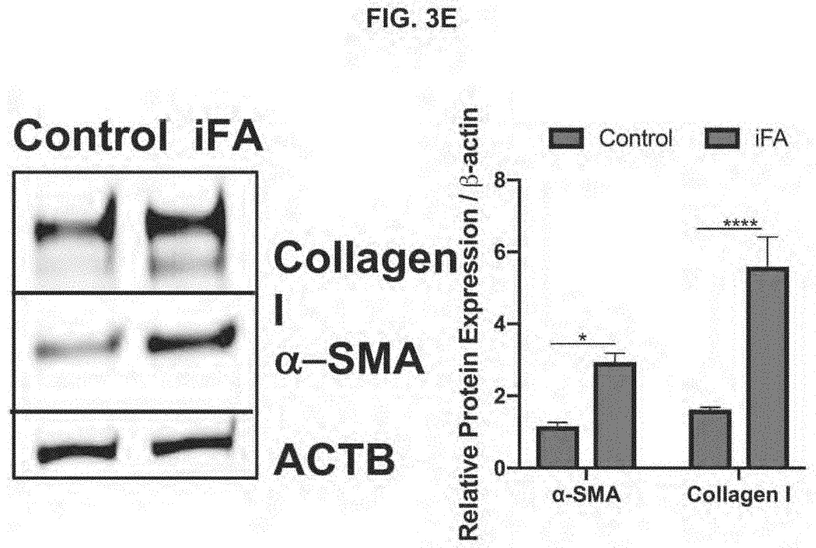

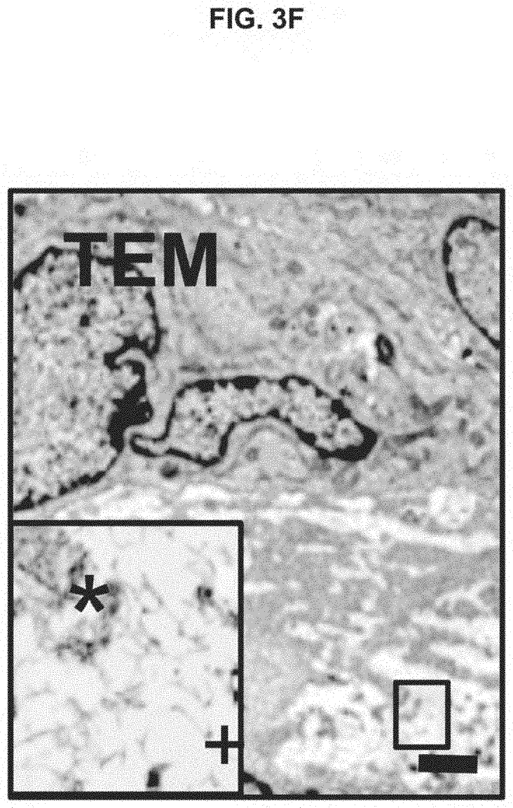

FIG. 3A shows phase-contrast images demonstrating propagation of mesenchymal-like cells on 13 kPa hydrogels (iFA) over time that reveal progressively increasing aggregate size with progression from D2 to 10; Scale bar, 50 μm. FIG. 3B shows phase-contrast images demonstrating the development of the iFA phenotype only in cultures from mesenchymal-like cells. Primary fibroblasts failed to generate the phenotype irrespective of the parent source. Scale bar, 50 μm.

FIG. 3C shows quantification of EdU positive cells in primary fibroblasts and mesenchymal-like cells grown on 13 kPa hydrogels related to FIG. 2B. All data are presented as mean±s.e.m; ****P<0.0001 using 2-way ANOVA followed by Tukey's multiple comparisons test. FIG. 3D shows fibrosis-related genes expression by qPCR in primary fibroblasts (control) and mesenchymal-like cells (iFA) cultured on 13 kPa hydrogels (n=5).

FIG. 3E shows representative immunoblot analysis of the expression of Collagen I and α-SMA in primary fibroblasts (control) and mesenchymal-like cells (iFA) showing increased expression of the fibrosis-related proteins in the iFA cultures collected at day 13 (left panel). Beta actin was used as a loading control. Right panel shows quantitative data presented as mean±s.e.m; ****P<0.0001, *P<0.05 using 2-way ANOVA followed by Sidak's multiple comparisons test. FIG. 3F shows representative transmission electron microscopic (TEM) images showing ultrastructure of cells in the iFA model amidst copious amounts of matricellular proteins (asterisk) and fibrillar proteins (plus). Inset is a higher magnification of the TEM image; Scale bar, 1 μm.

### Activated PAI-1 Activity

Quantitation of biologically active TGF-β levels was performed using mink lung epithelial cells stably transfected with plasminogen activator inhibitor-1 promoter/luciferase construct, in which luciferase activity represents bioactive TGF-β levels according to established protocols (Mazzieri et al., 2000 Methods in molecular biology 142, 13-27). Briefly, cells in the iFA model were serum starved for 30 hrs and conditioned media was collected at different time points. 2.5×10⁴ mink lung epithelial cells were seeded per well in a 96-well plate and allowed to adhere to the plate for 3 hrs. The medium was then replaced with conditioned media from the iFA model. Separately, control medium containing increasing concentrations (long/ml-30 pg/ml range) of rTGF-β1 was used to generate a standard curve. After 24 hrs of incubation at 37° C., cells were lysed with equal quantity of Bright-Glo™ Luciferase Assay System (Promega) and luminescence was measured using a DTX 880 multimode detector. The luciferase activity that was recorded as relative light units (RLU) was interpolated to TGF-β1 activity (pg/mL) using the TGF-β standard curve.

FIG. 4A shows active TGF-β secreted during the progression of the iFA phenotype (D2 to 14) was quantified using conditioned medium from the cultures in a TGF-β bioassay with mLEC-PAI-1-Luc reporter cells. The graph depicts increasing levels of active TGF-β in the iFA model (n=3). mLEC, Mink Lung Epithelial Cell; PAI-1, Plasminogen Activator Inhibitor-1. FIG. 4B shows time-dependent levels of secreted TGF-β1 during the development of the iFA phenotype (D4 to 13). A significant increase in secreted TGF-β1 protein in the iFA model was observed over time similar to that reported in fibrotic organs (n=6). Data represent mean±s.e.m; **P<0.01 using 2-way ANOVA and Sidak's multiple comparisons test.

FIG. 5A shows PAI-1 promoter/luciferase construct-transfected mink lung epithelial cells that were incubated with various concentrations of human recombinant(r) TGF-β1 at 37° C. for 20 h. The standard curve showed a dose-dependent increase in luciferase activity (relative light units, RLU) by rTGF-β1 between 0 and 10 ng/ml. The standard curve was used to determine the bioactivity of TGF-β in the iFA model. FIG. 5B shows time-dependent levels of secreted TGF-β1 during the development of the iFA phenotype (D4 to 13). A significant increase in secreted TGF-β1 protein in the iFA model was observed over time similar to that reported in fibrotic organs (n=6). Data represent mean±s.e.m; **P<0.01 using 2-way ANOVA and Sidak's multiple comparisons test.

### Assessing Proliferation by EdU Labeling

Cells were incubated with 10 μM EdU (Invitrogen) at specified time points of culture for 6 hrs, fixed with 4% PFA, and detected by staining with Alexa594-azide according to the manufacturer's instructions. The cells were additionally counterstained with Vimentin and/or α-SMA and DAPI.

### Decellularization of Primary Fibroblasts and iFA Model

Coverslips were gently washed with PBS and subjected to repetitive 30-minute freeze-thaw cycles in water, six times in total. The water was then replaced with 25 mM ammonium hydroxide containing 0.5% Triton X-100 and incubated at room temperature for 15 minutes. The coverslip was then washed twice with PBS and fixed with 4% PFA for 10 minutes and processed for immunostaining.

### Multiplex Analysis of Cytokines

The Milliplex human cytokine/chemokine Panel IV, human TIMP Panel 2, and human TGFβ 1,2,3 magnetic bead kits (EMD Millipore) were used per manufacturer's instructions. Prior to plating, all samples tested for human TGFβ only were activated and neutralized with 1.0 N HCl and 1.0 N NaOH, respectively. Briefly, 25 μl of undiluted or treated cell culture supernatant samples were mixed with 25 μl of magnetic beads and allowed to incubate overnight at 4° C. while shaking. After washing the plates with wash buffer in a Biotek ELx405 washer, 25 μl of biotinylated detection antibody was added and incubated for 1 hour at room temperature while shaking. 25 μl of streptavidin-phycoerythrin conjugate was then added to the reaction mixture and incubated for another 30 minutes at room temperature while shaking. Following additional washes, beads were resuspended in sheath fluid, and fluorescence was quantified using a Luminex 200 instrument. Similarly, a custom magnetic Luminex assay kit of select analytes (R&D Systems) was used per manufacturer's instructions. Cell culture supernatants were prepared with a 2-fold dilution. 50 μl of diluted sample were added to 50 μl of provided micro-particle cocktail and incubated for 2 hours at room temperature while shaking. After washing with wash buffer, 50 μl of biotin antibody cocktail was added and incubated for 1 hour at room temperature while shaking. Following additional washes, 50 μl of diluted Streptavidin-PE was then added for incubation for 30 minutes at room temperature while shaking. The plate was then washed again and the micro-particles resuspended in 100 μl wash buffer. After an additional 2-minute incubation while shaking, the data was acquired using a Luminex 200 instrument.

For all multiplex assays, data was analyzed using MILLIPLEX Analyst 5.1 software. Kits used were TGFβ 1,2,3 (MILLIPORE: TGFBMAG-64K-03), TIMP Panel 2 (MILLIPORE: HTMP2MAG-54K), Cytokine/Chemokine Panel IV (MILLIPORE: HCY4MG-64K-PX21); Custom Luminex (R&D: CUST01704 13). Multiplexing LASER Bead Technology service from Eve Technologies Corp (Calgary, Canada) was used to simultaneous analyze several cytokines, chemokines and growth factors in a single assay from the iFA model at specified time points using the Human Cytokine Array/Chemokine Array 64-Plex Discovery Assay based on a MILLIPLEX® MAP assay kit from Millipore according to their protocol. The assay sensitivities of these markers range from 0.1-55.8 pg/ml.

FIG. 6A shows a representative image of the iFA model at D13 of culture, demonstrating senescent cells with SA-β-Gal staining. FIG. 6B shows cytokine levels determined from conditioned media of the iFA model at D13 relative to D4 (n=5 in duplicate). Scale bars, 50 μm. Data in figures represents the mean±s.e.m; ****P<0.0001 ***P<0.001 **P<0.01 *P<0.05 using 2-way ANOVA and Sidak's multiple comparisons test. FIG. 6C shows HMGB1 levels determined from conditioned media of the iFA model at D13 relative to D4 (n=5 in duplicate). Scale bars, 50 μm. Data in figures represents the mean±s.e.m; ****P<0.0001 *** P<0.001 **P<0.01 *P<0.05 using 2-way ANOVA and Sidak's multiple comparisons test.

FIG. 6D shows a representative image of the iFA model at day 13 stained for NF-κB revealing its nuclear localization that is upstream of the inflammatory cytokine expression seen in the model. HMGB1 was translocated from the nucleus to the cytoplasm. Scale bars, 50 μm.

FIG. 7 shows cytokine profiles of proteins secondary to acute-phase response in supernatants from the iFA model at D13 relative to D4 cytokines in AA5r versus DMSO, and AA5p versus DMSO-treated cells (n=4 in duplicate). Data represents the mean±s.e.m ****P<0.0001, **P<0.01, *P<0.05 using one-way ANOVA and Dunnett's multiple comparison test.

### Quantification of Secreted HMGB1 and NPTX1

Conditioned media at different time points of progression of the phenotype, and on prevention and resolution drug treatments were collected and quantification of protein was performed using the IBL man HMGB1 ELISA kit or LSBio NPTX1 ELISA kits per manufacturer's instructions. 10 μl of each supernatant sample was added to anti-HMGB1 polyclonal antibody-coated plate and incubated for 20 hours at 37° C. All liquid was then removed and the plate manually washed 5 times with wash buffer. 100 μl of enzyme conjugate was then added and incubated for 2 hours at room temperature. After washing again, color solution was added at 100 μl per well and incubated for 30 minutes at room temperature. Following an additional five washes, the plate was incubated with 90 μl of TMB substrate for another 15 minutes at 37° C. in the dark. The reaction was then stopped by addition 100 μl of stop solution. Absorbance was measured at 450 nm using a SpectraMAX Plate Reader. Data was analyzed using SoftMax Pro software.

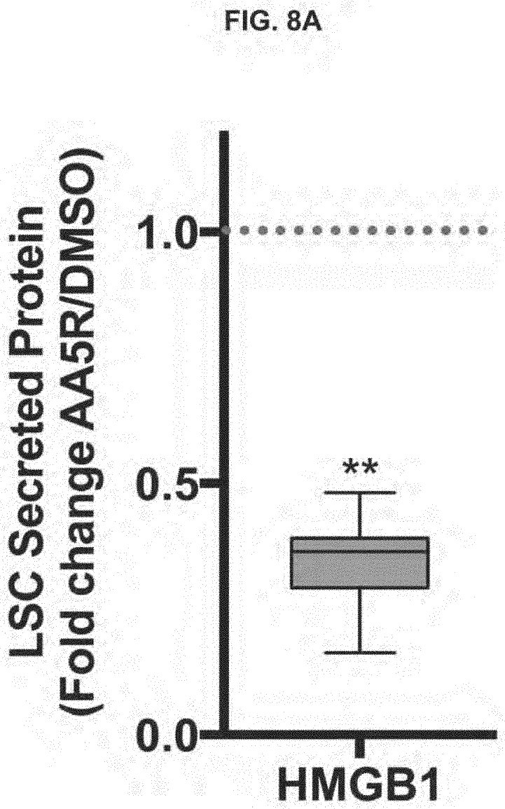

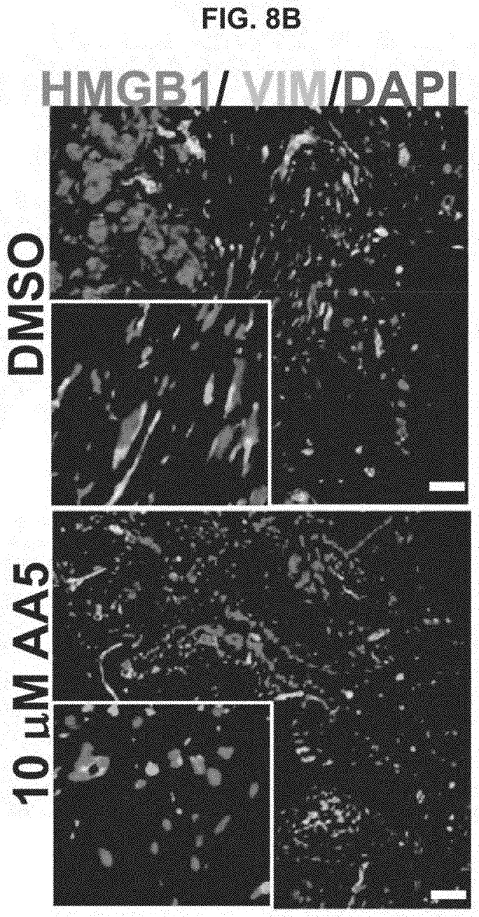

FIG. 8A shows relative secreted levels of HMGB1 in supernatants from the conditioned media of AA5-treated LSC cultures compared to DMSO-treated controls 48 hours after treatment (n=9) were quantified by Luminex assay revealing a significant decrease in secreted HMGB1. Data represent min-max and median protein abundance in AA5 treated LSCs relative to DMSO treated controls (red line). ** P<0.01 using two-way ANOVA and Sidak's multiple comparison test.

FIG. 8B shows a representative image of DMSO- and AA5-treated LSC stained for HMGB1 revealed almost no secreted HMGB1 in the AA5-treated samples within 48 hours of treatment in comparison to the DMSO controls. The samples were counter-stained for VIM and DAPI. Insets are higher magnified images; Scale bar, 50 μm.

FIG. 9 shows representative DMSO-treated, AA5-prevention (AA5p) and AA5-resolution (AA5r) iFA cultures immunostained for VIM and α-SMA (top panels) and Collagen I and α-SMA (bottom panels). Remnants of the scar were still visible among the monolayer of fibroblast-like cells; Scale bar, 50 μm.

### In Situ Cell Elasticity Measurements Using Atomic Force Microscopy (AFM)

Tapping mode AFM using the Bruker BioScope Catalyst Atomic Force coupled with Zeiss LSMS Confocal Fluorescence Microscope was used on the cells at 37° C. in cell culture media. AFM deflection images of cells were used in the imaging experiment. In the force measurement, sharp silicon nitride AFM probes (tip radius, 20 nm) were employed (Bruker Corp., USA). The spring constants of AFM tips were calibrated to be 0.10-0.11 N m⁻¹ and deflection sensitivities were 45-50 nm V⁻¹, using Thermo K Calibration (Agilent Technologies, USA). The approaching/retracting speed of the AFM tip during the force curve measurement was 6 μm s⁻¹. Force-distance curves were recorded to obtain cell elasticity (Young's modulus, E) of individual cells. For each time point, at least 20 single cells, 20 cells at the base of the iFA and 20 cells at the tip of the scar were measured with over 15 force-distance curves per cell to obtain significant results. The Young's modulus was calculated via the Scanning Probe Image Processor (SPIP) software (Image Metrology, Denmark) by converting the force-distance curves to force-separation curves and fitting the Sneddon variation of the Hertz model, which describes conical tips indenting elastic samples.

FIG. 10A shows a representative phase contrast image (top left) of the iFA model at D13 that was used to measure the elastic modulus. Arrows point to representative regions of the culture where the measurements were made. 1 refers to single cells in the dish. 2 refers to the cells on the periphery of the iFA phenotype. 3 refers to the center of the iFA phenotype. 3D rendering of the AFM amplitude channel that shows representative areas of 1,2 and 3 from panel (top right and bottom panels).

FIG. 10B shows force versus distance curves measured on cells from FIG. 6A. The black line depicts the curve obtained on the stiff petri dish. The magenta line represents curve obtained from cells in the iFA and blue line represents single cells. The measured indentation was fitted to the Sneddon model. Elastic moduli of iFA cells and single cells were calculated as 30 kPa and 15 kPa, respectively.

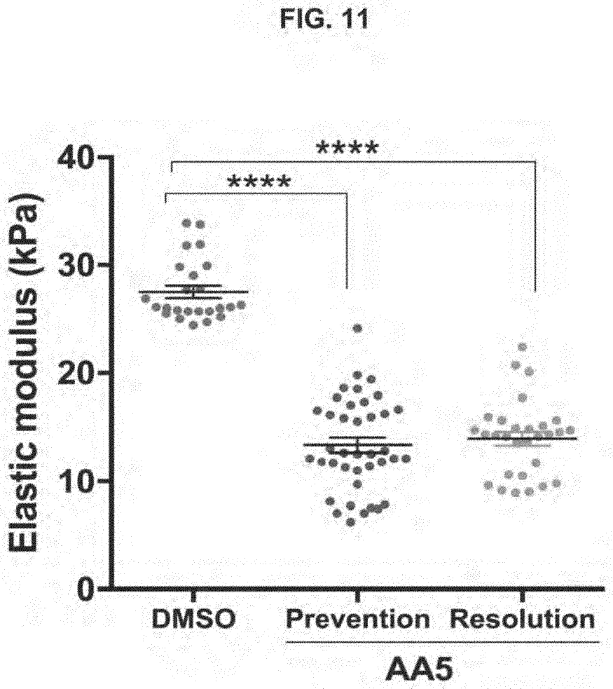

FIG. 11 shows quantification of stiffness of the cells in the DMSO-treated, AA5p and AA5r iFA model.

Fibrosis Models with the Exogenous Addition of TGF-β

Hepatic Stellate Cell (LX-2) was purchased from Millipore. Primary skin, healthy lung and IPF lung fibroblasts were prepared from punch biopsies collected from patient samples according to the Institution's IRB approvals. Three patient lines were used for each experiment. 100,000 cells were seeded in a 35 mm dish and allowed to grow to confluency of 48 hours. After a 24 hour serum starvation, the media was replaced with serum-free media containing either 2 ng/ml rhTGF-β for the LX-2 cell line or 10 ng/ml rhTGF-β for all other primary cultures. Untreated cultures in serum free were maintained as controls. After 48 hours with daily media changes, samples were collected for either RNA or protein analysis.

FIG. 12A shows a heat map summarizing fold change for 84 fibrosis-related genes exhibiting differential expression across the iFA model (D13 vs D4) and TGF-β-induced fibrosis models (TGF-β-treated versus untreated); SF, skin fibroblasts; LF, normal lung fibroblasts; IPF-LF, IPF lung fibroblasts; LX-2, Hepatic stellate cell line. Heat maps show log base 2-transformed data for each experiment mean expression (n=3), of genes with p value <0.05 in at least one of the samples per gene.

FIG. 12B shows a densitometric analysis depicting fold change of α-SMA and Collagen I in fibrosis models (TGF-β-treated versus untreated) and iFA model (D13 vs D4) analyzed by immunoblotting. The intensity of individual bands was normalized to total protein (for α-SMA) or β-actin (for Collagen I). Data represented as the mean±s.e.m ****P<0.0001, ***P<0.001, **P<0.01 using two-way ANOVA and Sidaks's multiple comparison test.

FIG. 12C shows a heat map summarizing fold change of differential expression of cytokines and growth factors in supernatants of fibrosis models (TGF-β-treated versus untreated) and iFA model (D13 vs D4) (n=3 in duplicate). Heat maps show log base 2-transformed data for each experiment mean expression (n=3 in duplicate), of genes with p-value <0.05 in at least one of the samples per protein.

FIG. 12D shows HMGB1 levels determined from conditioned media in the fibrosis models (with and without TGF-β treatment) and the iFA model at D13 relative to D4 (n=6). Data represents the mean±s.e.m; ****P<0.0001 using 2-way ANOVA and Sidak's multiple comparisons test.

FIG. 13A shows a representative immunoblot analysis of the expression of Collagen I and α-SMA in exogenously TGF-β-treated fibrosis models of skin (SF), lung (LF, IPF_LF) and liver (LX-2) compared to the iFA model at Day (D) 2 and 13 with no addition of TGF-β. Total protein (α-SMA) and ACTB (Collagen I) were used as a loading controls.

FIG. 13B shows representative IF images of Collagen I and α-SMA in exogenously TGF-β-treated fibrosis models of skin (SF), lung (LF, IPF_LF) and liver (LX-2) compared to the iFA model at Day (D) 2 and 13 with no addition of TGF-β. Scale bars, 50 μm. FIG. 13C shows representative images of exogenously TGF-β-treated fibrosis models of skin, lung and liver compared to the iFA model during the progression of the fibrotic phenotype the iFA model at day (D) 2 to 13 of culture, demonstrating senescent cells with SA-β-Gal staining; Scale bars, 50 μm.

### Phenotypic High-Content Drug Screen

A phenotypic high-content drug screen was prepared to identify compounds capable of preventing the iFA phenotype in a 96-well format. An ImageXpress XL high-throughput imager with a 4× Plan objective (N/A 0.20) was used with image based focusing. Briefly, 100 μL media were plated in each well of 96 well plate using a Thermo Multidrop non-contact dispenser. Using a custom V&P pin tool mounted to a Beckman FX liquid handler, 1.5 μL compounds or DMSO were added. Mesenchymal-like cells were added as a suspension using the Multidrop at a density of 3.5×10³ cells/well. The resulting compound concentration was 10 μM. Cells were imaged after 7 days of incubation at 37° C. and 5% CO₂: A final of 0.5 μg/ml of Calcein AM and/or 10 μg/ml propidium iodide (viability dye) with 1 μg/ml Hoechst 33342 were added and imaged using the ImageXpress XL system. Data mining was performed using a custom module consisting of the following steps: The image background was removed using a top-hat filter with 25 μm round shape polisher. Next, cellular aggregates and individual spindle-shaped cells were identified. Aggregates of cells were defined by size with a cut-off of 60 to 200 micron and an intensity of at least 200 gray scales over background. Individual spindle-shaped cells were identified as smaller objects with cut-offs from 15 to 100 micron and a lower intensity of at least 70 gray scales over background.

Additional morphometric measurements were made by counting labeled cell nuclei and measuring total area covered with cells per well. The data resulting from the screen were batch exported through MetaXpress and uploaded into a Collaborative Drug Discovery (CDD) cloud based database (Ekins and Bunin, 2013, Methods in molecular biology 993, 139-154.). Hits were selected on a cut-off of more than 3 standard deviations from mean using data normalized to reference wells in each plate. IC50 values (the half maximal inhibitory concentration) from confirmation experiments were calculated from dose-response signal curves using the Prism software (GraphPad Software).

Signal-to-background (S/B) ratio, standard variation and variation coefficients were calculated as the signal of DMSO treated wells divided by the signal of the hit compound (AA5) treated wells. Z′ values were used to measure assay quality which was calculated by the formula of Z′=1−3(SDTotal+SDBackground)/(MeanTotal−MeanBackground)(Inglese et al., 2006) where SDTotal and MeanTotal are the standard deviation and mean of signal for DMSO treated wells, and SDBackground and MeanBackground are the standard deviation and mean of signal for AA5 treated wells. Data normalization and curve fitting were performed as previously reported (Kariv et al., 1999, J Biomol Screen 4, 27-32.). Z′ values were obtained for the number of cellular aggregates and also calculated a phenotypic cell index as the product of cell number x area/Log(number of iFA aggregates+5). Secondary screening using α-SMA and collagen deposition was employed to further ensure efficacy and resolution of the iFA phenotype of the compounds. For the resolution screen, cells were seeded in a 96-well plate as mentioned above and compounds were added at day 13 to analyze their capacity to resolve the iFA phenotype using the same readouts as above. Dose response experiments were carried out as in the secondary screen but with at least 12 steps of a 1:1 dilution resulting in final compound concentrations from 500 μM to high nanomolar ranges.

FIG. 14 shows a superimposed representative scatter plot showing the results from a single 96-well plate of the iFA-prevention assay. Green dots represent the total number of live cells per well analyzed according to the parameters listed in FIG. 15. Red dots represent the total number of cellular aggregates in each well analyzed according to the parameters listed in the table in FIG. 15. All plates contained DMSO controls in wells A1 through H1 and A12 through H12. The black line represents the statistical cut-off of the DMSO vehicle control used for selecting primary hits (≥80% viability). A hit molecule would be identified as one that has no cellular aggregates (red dot is near/at 0) and cell viability greater than 80% (green dot is above the cut-off line). FIG. 15 shows high content staining discrimination of cellular aggregates vs individual spindle-shaped live cells. Cells are stained with Calcein-AM vital dye. Table shows the parameters that were used to identify and count cellular aggregates and total number of individual spindle-shaped live cells. Scale bar, 750 μm.

FIG. 16A shows prevention of the iFA phenotype in the well containing AA5 compared to DMSO control. Bottom panels are higher magnifications of boxed areas in the top panel. Scale bar, 750 μm. FIG. 16B shows the dose response of AA5 showing IC₅₀ of FIG. 17A shows representative images from wells of the iFA model treated with 0.6 μM-10 μM AA5. Cells were stained with viability dye Calcein AM and the nuclei were counterstained with Hoechst 33342. Full prevention of phenotype was seen at low micromolar concentrations; Scale bar, 750 μm. FIG. 17B shows comparative IF images of DMSO (top panel) and AA5-prevention (bottom panel) treated iFA model from days (D) 3, 5, 7, and 9 in culture labeled with EdU for 6 hours. The cells were counterstained with VIM and DAPI. Scale bar, 50 μm.

FIG. 17C shows EdU positive DAPI cells from FIG. 17B quantified for each time point. Treatment with AA5 did not display any significant difference in the proliferation rate when compared to the DMSO treated cells. All data are presented as the mean±s.e.m; non-significant data using one-way ANOVA and Tukey's multiple comparison test. FIG. 17D shows a representative immunoblot analysis of the expression of Collagen I and α-SMA in the DMSO and AA5-prevention samples at D8. ACTB was used as a loading control. FIG. 17E shows the time-dependent fold change in secreted POSTN and TIMP-3 in response to AA5-prevention treatment versus DMSO in the development of the iFA phenotype (D3 to D13).

Table 1 shows compounds identified in the library screen that exhibited efficacy in both primary and secondary testing. Several compounds showed a dose response and reversal of fibrosis. The primary assay involved plating together the iPSCs and Table 1 compounds sourced from the library on day 1 on the hydrogel and examining the phenotype on day 7 of the culture as described herein. The secondary screen repeated the assay using compound sourced commercially or from additional library materials. The dose response refers to a 20 point dose course that ranges from picomolar to millimolar concentrations. Reversal of fibrosis refers to allowing the phenotype to form over 10 days in the dish and then compounds were added to evaluate whether a change in phenotype occurred with cells moving away from the scars and spreading out on the dish instead of the scars becoming larger. As described herein, the cellular phenotype parameters evaluated included size, fluorescence intensity of the iFA phenotype, the shape factor of the cells/aggregates, and the cells' viability in the presence of viability dyes (Calcein AM).

TABLE 1

| | Efficacy in | Efficacy in | | Reversal |
|---|---|---|---|---|
| | primary | secondary | Dose | of |
| Compound name | screen | screen | response | fibrosis |
| 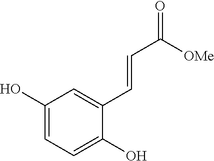 [SMILES: COC(=O)/C=C/c1cc(O)ccc1O] | +++ | ++ | ++ | ND |
| Methiopropamine | +++ | +++ | +++ | Yes |
| AA5 | +++ | +++ | +++ | Yes |
| D-methionine | +++ | ++ | ND | ND |
| (−)Eseroline fumarate salt. | +++ | ++ | ND | ND |
| Ciclopirox ethanolamine. | +++ | ++ | ND | ND |
| Fenbendazole. | +++ | ++ | ND | ND |
| 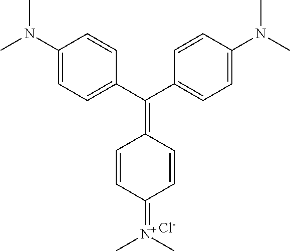 | +++ | ++ | ND | ND |
| CEP-33779 JAK2 Inhibitor | +++ | ++ | ND | ND |
| Y-27632 Rock Inhibitor | +++ | ++ | ND | ND |
| Recombinant Human IFN-gamma Protein | +++ | ++ | + | ND |
| ±)-2-Amino-3-phosphonopropionic acid | +++ | + | ND | ND |
| γ-Aminobutyric acid | +++ | + | ND | ND |
| Bupropion hydrochloride | +++ | + | ND | ND |
| Arvanil | +++ | + | ND | ND |
| CGP-7930 | +++ | + | ND | ND |
| CGP-13501 | +++ | + | ND | ND |
| Chlorpropamide | +++ | + | ND | ND |
| Cyclophosphamide | +++ | + | ND | ND |
| 1-(4-Chlorobenzyl)-5-methoxy-2- | +++ | + | ND | ND |
| methylindole-3-acetic acid | | | | |
| 1-Pheny1-3-(2-thiazolyl)-2-thiourea | +++ | + | ND | ND |
| Tyrphostin 51 | +++ | + | ND | ND |

+ = weak effect
++ = moderate effect
+++ = strong effect
ND = not determined

Weak effectors displayed at least a noticeable effect on surface area resolved fibrosis; modest effectors displayed at least approximately a 33% reduction in surface area resolved fibrosis; and strong effectors displayed at least approximately a 66% reduction in surface area resolved fibrosis.

### RNA Preparation and Expression Analysis

In-vitro cultures of PF and cells in the iFA model were washed once with PBS and total RNA from the samples were extracted using the RNeasy Mini Kit (Qiagen) according to the manufacturer's instructions. For the lung slice cultures (LSC), tissues were snap-frozen after treatment and stored at −80° C. until RNA isolation. Lung slices were homogenized using a handheld homogenizer and passing the homogenate through a Qiashredder (Qiagen). Total RNA from the LSC and cells from the disease model were extracted using the RNeasy Mini Kit (Qiagen) according to the manufacturer's instructions. The RNA concentrations were measured on a NanoDrop ND-1000 spectrophotometer. Single-stranded cDNA was synthesized from 200 ng of total RNA using Superscript IV and random hexamer primers (Invitrogen) in a volume of 20 μl. cDNA was then used for qRT-PCR analysis. PCR reactions were performed using Taqman Gene Expression Assay mix (Applied Biosystems) according to the manufacturer's instructions. Taqman probes are listed in Table 2.

TABLE 2

| Gene Name | TaqMan Assay ID |
|---|---|
| ACTA2 | Hs00426835_g1 |
| COL1A2 | Hs01028956_m1 |
| TGFB1 | Hs00998133_m1 |
| TGFB3 | Hs01086000_m1 |
| TIMP1 | Hs01092512_m1 |
| MMP3 | Hs00968305_m1 |
| PLAT | Hs00263492_m1 |
| LOX | Hs00942480_m1 |
| PTX3 | Hs00173615_ml |
| NPTX1 | Hs00982601_m1 |
| CD163L1 | Hs00264549_m1 |

qRT-PCR reactions were performed using the StepOnePlus (Applied Biosystems). Relative gene expression was calculated using the 2^{-ΔΔCt} method, with 18S Cat. #4331182 (Invitrogen) as housekeeping gene. For the RT²qPCR arrays, cDNA from DMSO and AA5 treated iFA and was added to the RT² qPCR iTaq Universal SYBR green Master Mix (Biorad). 20 μl of the experimental cocktail was added to each well of the Fibrosis PCR (Qiagen). Real-Time PCR was performed on the StepOnePlus qPCR system (Applied Biosystems) using SYBR green detection according to the manufacturer's recommendations. All data from the PCR was collected and analyzed by SA Bioscience's PCR Array Data Analysis Web Portal.

FIG. 18A shows secreted levels of HMGB1 in the iFA model during AA5p and AA5r treatments in comparison to DMSO-treated controls measured in cell culture supernatants. Significantly decreasing cell stiffness in (b) and HMGB1 levels in (c) are represented as the mean±s.e.m ****P<0.0001, **P<0.01 using one-way ANOVA and Dunnett's multiple comparison test.

FIG. 18B shows ClueGO clustering analysis results of up- (red) and down-regulated (blue) genes in a pairwise comparison of AA5p treatment in a DMSO-treated iFA model. p-value of ≤0.05 are shown. FIG. 18C shows ClueGO clustering analysis results of up- (red) and down-regulated (blue) genes in a pairwise comparison of AA5r treatment in a DMSO-treated iFA model. p-value of ≤0.05 are shown.

FIG. 18D shows a comparison of gene expression fold change levels of long-pentraxin family members PTX3 and NPTX1 and scavenger receptor CD163L1 in the AA5r and AA5p treated iFA model when compared to the DMSO treated iFA cultures (n=5); data represent the mean±s.e.m; ****P<0.0001 using two-way ANOVA and Sidak's multiple comparison test.

FIG. 18E shows the time-dependent fold increase in secreted PTX3 and NPTX1 on AA5p treatment measured in supernatants collected on D4, 7 and 13 of treatment when compared to DMSO controls in the iFA model (n=4). Data represent the mean±s.e.m; ****P<0.0001 using two-way ANOVA and Tukey's multiple comparison method.

FIG. 19A shows the effect of AA5 on preventing fibrosis in the production of a shift in the gene expression levels that is comparable to the differential expression of genes in the lung IPF tissue (n=7) in contrast to healthy lung controls (n=8). FIG. 19B shows the effect of AA5 on resolving fibrosis in the production of a shift in the gene expression levels that is comparable to the differential expression of genes in the lung IPF tissue (n=7) in contrast to healthy lung controls (n=8).

FIG. 19C shows a list of significantly enriched terms using ClueGO analysis of the AA5-mediated prevention iFA phenotype in the iFA model. FIG. 19D shows a list of significantly enriched terms using ClueGO analysis of the AA5-mediated resolution iFA phenotype in the iFA model. Terms are grouped according to the functional group that they belong to. Terms that are part of more than one functional group are shown in purple. The group p-values (corrected with Bonferoni step down) are indicated in between the bar charts. The bars indicate the percentage of the up- (red) or down-regulated (blue) genes per each term. The numbers outside the bars show the actual number of genes associated with the specific terms, while the numbers in the parenthesis show the individual term p-value.

### RNA Sequencing Analysis of Step-Wise Progression, iFA (AA5) and LSC

Libraries for RNA-Seq were prepared with the Nugen human FFPE Kit (LSC). The workflow consisted of cDNA generation, end repair to generate blunt ends, adaptor ligation, adaptor cleavage and PCR amplification. Different adaptors were used for multiplexing samples in one lane. Sequencing was performed on the Illumina Nextseq500 with a single read 75 run.

Aggregate cultures of iSDCs (iFA model, day4 and day13), IPF whole lung tissue from the previously published data (Koyama et al., 2013, Stem cells and development 22, 102-113.) and iFA model post-AA5-prevention and AA5-resolution treatment were analyzed using the following method. The reads were aligned to the NCBI build 37.2 transcript set using Bowtie2 version 2.1.0 and TopHat version 2.0.9. TopHat's read alignments were assembled by Cufflinks version 2.2.1. Technical replicates were combined and aligned together in all cases except for the AA5-treated model. The gene expression estimates were then log 2-transformed and genes with no reads across all samples were removed from further analysis.

LSC from IPF patients treated with AA5 or DMSO for 48 hours were analyzed using the following method. Data quality check was performed on Illumina SAV. Demultiplexing was performed with Illumina Bcl2fastq2 v 2.17 program. The reads were first mapped to the latest UCSC transcript set using Bowtie2 version 2.1.0 and the gene expression level was estimated using RSEM v1.2.15. TMM (trimmed mean of M-values) was used to normalize the gene expression. The gene expression estimates were then log 2-transformed and genes with no reads across all samples were removed from further analysis.

FIG. 20 shows the fold change of gene expression of ACTA2, COL1A2 and TGF-β3 in AA5-treated lung slice cultures (LSC) relative to DMSO-treated controls 48 hours after treatment (n=4). Data represent mean±s.e.m ***P<0.001 **P<0.01 *P<0.05 using two-way ANOVA and Sidak's multiple comparison test.

### Gene Set Enrichment Analysis (GSEA)

Canonical pathways GSEA (Mazzieri et al., 2000, Methods in molecular biology 142, 13-27) was performed using 1,188 canonical pathways (CP) defined by the Broad Institute's Molecular Signatures Database (MSigDB). Gene sets with less than 10 genes were excluded from the analysis. For calculating the normalized enrichment scores (NES), genes were ranked based on the signal-to-noise ratio. After enrichment results from the IPF whole lung tissue and iFA model were obtained, gene sets were ordered based on the average NES rank between the two analyses.

### The Rank Rank Hypergeometric Overlap (RRHO) Analysis

The rank rank hypergeometric overlap (RRHO) (Plaisier et al., 2010) was calculated using the web application (http://systems.crump.ucla.edu/rankrank/rankranksimple.php). The step size of 100 was used to bin the ranked items to improve the run time of calculating the hypergeometric distribution. Genes were ranked based on their differential expression in comparison groups using a log 10-transformed t-test P-value with the sign denoting the direction of change.

FIG. 21 shows the Rank Rank Hypergeometric Overlap (RRHO) analysis that indicates statistically significant hypergeometric overlap between differentially expressed genes in the iFA model post AA5-prevention (n=2) and post AA5-resolution (n=2) treatments, as compared to the untreated control, iFA (n=2). See also FIGS. 19A-D.

### Functional Network Analysis

The gene list of interest in each analysis was compiled by pairwise generating log 2-transformed gene expression fold changes between AA5-treated and untreated cases. Only log 2-transformed fold changes greater than 1.3 and less than −1.3 were considered significant for further analysis. From the resulting matrix, a co-expression correlation matrix was calculated and genes were ranked based on how often they had correlation greater than 0.7. For AA5-prevention-treated (AA5-resolution-treated) iFA model 54 (82) genes up-regulated and 58 (75) genes down-regulated after AA5 treatment were included in the network analysis. For AA5 treated lung slice cultures 68 genes up-regulated and 57 genes down-regulated after AA5 treatment were included in the network analysis.

A Cytoscape plug-in ClueGo (Bindea et al., 2009, Bioinformatics 25, 1091-1093) was used to visualize functionally grouped terms that contained genes from the list of interest in the form of network. The terms in Biological Processes, Cellular Component, Immune System Process and Molecular Function Gene Ontologies from the February, 2017 version were analyzed. Terms sharing genes were linked together into functional groups based on kappa score level ≥0.4. Only terms with two-sided hypergeometric p-value ≤0.05 were kept.

### In Vivo Efficacy of AA5 Using Murine Model of Ocular Mucosal Fibrosis

Female C56/B17 mice 8-10 week of age were used for all experiments. The mice were housed in the institute's vivarium in compliance with the Animal Research Committee. Immune-mediated conjunctivitis was induced in by i.p. injection of 200 μl of immunization mix containing: 10 μg Ovalbumin (OVA) (Sigma-Aldrich), 4 mg aluminium hydroxide (Thermo Scientific), and 300 ng of pertussis toxin (Sigma-Aldrich). After 2 weeks, mice received topical OVA challenge (250 mg) once a day for 7 days for the development of fibrosis. To evaluate the efficacy of AA5 in preventing fibrosis, topical OVA challenge was accompanied by addition of 1, 10, 100 or 1000 ug/mouse (n=12) AA5 administered in both eyes in a volume of 5 ul for a total of 7 days.

To evaluate the efficacy of AA5 in resolving fibrosis, topical OVA challenge was continued for an additional 7 days (days 14-21) after the initial OVA sensitization for the development of fibrosis. From day 21 to 28, OVA challenge was accompanied by addition of 1, 10, 100 or 1000 ug/mouse (n=12) AA5. 1% DMSO administered in both eyes in a 5 □l volume of PBS was used as control. Both eyes were scored in the same manner. For the scoring, a cumulative score of eyelid swelling (out of 3) and tearing (out of 3) was taken. At the end of the experiment at day 21 (prevention studies) or day 2 (resolution studies), mice were sacrificed and whole eyes were collected, fixed in 10% (v/v) formalin and processed by the standard methods for paraffin embedding. Sections (5 μm) were stained with Gomori Trichrome and H&E and imaged. Tissue sections were reviewed by 4 independent observers, including two observers who were blinded to the groups—a pathologist and another researcher. Conjunctivae were dissected from whole eyes and either processed for RNA or hydroxyproline.

FIG. 22A shows schema illustrating the experiment to induce and treat ocular fibrosis in mice in a dose-dependent manner. Naive mice (n=12) without OVA challenge were also monitored in the same manner throughout the challenge period. FIG. 22B and FIG. 22C show the ocular surface inflammatory score in OVA-treated mice (n=12) treated daily with topical eye drops containing DMSO or (0.1-1000 μg) AA5 in fibrosis prevention (FIG. 22B) and resolution (FIG. 22C) studies. Tearing, eyelid swelling, and inflammation were used for the clinical score. AA5 ameliorated the fibrotic response in both the prevention (AA5p) and resolution (AA5r) studies in a dose dependent manner. Data represent mean±s.e.m of inflammatory score. **** P<0.0001 ***P<0.001 **P<0.01 *P<0.05 using one-way ANOVA and Sidak's multiple comparison test.

FIG. 22D and FIG. 22E show the total collagen content in conjunctival tissue in naïve, OVA-, DMSO- and AA5-prevention (FIG. 22D) or AA5-resolution (FIG. 22E) treated animals following ocular scarring, showing a significant decrease in collagen content post AA5 treatment in a dose dependent manner (n=12). Data represent min-max and median of collagen content relative to total protein content. **** P<0.0001 ***P<0.001 **P<0.01 *P<0.05 using one-way ANOVA and Sidak's multiple comparison test.

FIG. 22F shows representative Gomori Trichrome stained sections of whole eyes (n=12 per group) that revealed qualitatively decreased thickness of conjunctiva and collagen deposition in the AA5 (1000 ug) treated eyes (arrows), comparable to the naïve group, thereby validating the potential of AA5 to reverse ocular mucosal fibrosis. Scale bar: 50 μm.

### Ex Vivo Efficacy of AA5 Using Fibrotic Lung Slice Culture System

All human samples were obtained in accordance with institute IRB. Tissue for lung slice experiments was obtained from patients with Idiopathic Pulmonary Fibrosis (IPF) at the time of lung transplant. IPF lungs were cored using an 8 mm diameter core, and manually sliced to produce relatively identical slices. Lung slices were cultured for 48 hrs in DMEM/F12 supplemented with 10% FCS in a rocker culture system at 37° C. and 5% CO₂ in the presence of 1% DMSO (control) or 10 μM AA5 (efficacy treatment). 24 LSC samples were prepared for each treatment per patient sample. Media was collected every 24 hrs for luminex assays and replaced with fresh control and treatment medium for the specified incubation time. Samples were collected for both RNA isolation and paraffin embedding at 48 hrs.

FIG. 23A shows relative secreted levels of fibrosis-related proteins in supernatants of AA5-treated LSC cultures compared to DMSO-treated controls 48 hours after treatment (n=9). FIG. 8A shows relative secreted levels of HMGB1 in supernatants from the conditioned media of AA5-treated LSC cultures compared to DMSO-treated controls 48 hours after treatment (n=9) that were quantified by Luminex assay showing a significant decrease in secreted HMGB1. Data represent min-max and median protein abundance in AA5 treated LSCs relative to DMSO treated controls (red line). ** P<0.01 using two-way ANOVA and Sidak's multiple comparison test.

FIG. 23B shows representative FACS plots revealing expression of SSEA4+ population in LSC treated with DMSO or AA5. Inset shows quantitative data of SSEA4+ cells. Data represents mean±s.e.m ***P<0.001 using two-tailed paired t-test.

FIG. 23C shows ClueGO clustering analysis results of up- (red) and down-regulated (blue) genes in a pairwise comparison of AA5- versus DMSO-treated LSCs. Nodes represent specific terms from GO ontologies with node's size reflecting the level of the term's enrichment significance. Terms sharing genes are linked together into functional groups based on kappa score level (≥0.4, shown as edges or overlapping nodes). The groups that have functional similarities can partially overlap. The red to blue color gradient represents the proportion of genes from up- versus down-regulated genes within each term. Equal proportions of the two clusters are shown in gray. Only terms with a two-sided hypergeometric p-value ≤0.05 are shown. Selected terms from the functional group are annotated. FIG. 26E shows a list of terms in the ClueGo functional network.

FIG. 25A and FIG. 25B show the fold change of expression of PTX3, NPTX1 and CD163L1 in AA5-treated LSC relative to DMSO-treated controls 48 hours after treatment at mRNA (FIG. 25A; n=6) and secreted protein (FIG. 25B; n=6) levels (for secreted NPTX1 levels from individual samples, see FIG. 26E). Data represent min-max and median protein abundance in AA5-treated LSCs relative to DMSO-treated controls (red line); ****P<0.0001 ** P<0.01 using two-way ANOVA and Sidak's multiple comparison test.

FIG. 25C shows representative images from wells of the iFA model treated with DMSO, 10 μM AA5 or 3.75 μgm/ml IFNγ stained with Calcein AM and DAPI (left panel). Partial prevention of the iFA phenotype was observed with the IFNγ treatment. Scale bar, 750 μm. Right panel shows representative immunostained images for VIM and α-SMA from the corresponding cultures in the left panel. IF revealed a change in the fibrotic (iFA) phenotype and reduced α-SMA and Collagen I on treatment with AA5r and IFNγ when compared to DMSO-treated controls. Remnants of the scar were visible among the monolayer of fibroblast-like cells with both treatments.

FIG. 25D shows the relative fold change of secreted proteins in LSCs treated with 3.75 μg/ml rIFNγ for 48 hours in comparison to DMSO-treated controls (n=4). The pattern of secreted protein fold changes of the was similar to that observed on AA5 treatment. Data represent min-max and median protein abundance in IFNγ treated LSCs relative to DMSO treated controls (maroon line); ** P<0.01 using two-way ANOVA and Sidak's multiple comparison test.

FIG. 26A shows a representative image of DMSO- and AA5-treated lung slice cultures (LSCs) stained for the proliferation marker PCNA 72 hours after treatment depicting viability of tissue. Samples were counterstained for α-SMA and DAPI. FIG. 26B shows relative secreted levels of TGF-β proteins in supernatants of AA5-treated LSCs compared to DMSO-treated controls, 48 hours after treatment (n=9). Data represent min-max and median protein abundance in AA5-treated LSCs relative to DMSO treated controls (red line).

FIG. 26C shows gene expression analysis showing relative expression levels of fibrosis-related genes in LSCs treated with AA5 compared with DMSO treatment depicting the fibro-protective effect of AA5 (n=6). TMM (trimmed mean of M-values) was used to normalize the gene expression; ****P<0.0001 *** P<0.001 ** P<0.01 * P<0.05.

FIG. 24 shows the list of significantly enriched terms in the ClueGO analysis from the pairwise comparison between AA5 and DMSO treated LSCs. Terms are grouped according to the functional group that they belong to. Terms that are part of more than one functional group are shown in purple. The group p-values (corrected with Bonferoni step down) are indicated in between the bar charts. The bars indicate the percentage of the up- (red) or down-regulated (blue) genes per each term. The numbers outside the bars show the actual number of genes associated with the specific terms, while the numbers in the parenthesis show the individual term p-value.

FIG. 26D upper panel shows representative images of DMSO- and AA5 (10 μM)-treated LSCs stained for NPTX1 revealing excessive NPTX1 staining in the honey comb cyst areas of the IPF lung in the AA5-treated samples within 48 hours of treatment in comparison to the DMSO controls. The samples were counterstained for DAPI. Insets are higher magnified images. Scale bar, 50 μm. Lower panel shows representative images of DMSO- and AA5 (10 μM)-treated LSCs stained for scavenger receptor protein CD163L1 by immunohistochemistry and revealed prominent staining on macrophages in both treatments, but a higher expression of shed receptor staining was seen on AA5 treatment suggesting activation of the phagocytic cells. Insets are higher magnified images. Scale bar, 100 μm.

FIG. 26E shows secreted levels of NPTX1 in the LSCs treated for 48 hours with DMSO and AA5 from 5 patient samples in quadruplicate. While increasing amounts of NPTX1 secretion on AA5 treatment can be seen, these data from individual patient samples show an outlier that skewed the data towards non-significance. Data represent min-max and median protein abundance *** P<0.001 ** P<0.01 * P<0.05 using two-way ANOVA and Sidak's multiple comparison test.

FIG. 27A shows a representative still frames from a time-lapse series showing an invasive and progressive phenotype in the iFA model (upper panel), which resolved on addition of AA5 (lower panel); Scale bar, 100 μm. FIG. 27B shows quantitative data presented as mean±s.e.m depicting the expression of Collagen I and α-SMA in the iFA model and AA5-presolution cultures collected at day 16. Beta actin was used as a normalization control. n=3, **P<0.01, *P<0.05 using 2-way ANOVA followed by Sidak's multiple comparisons test.

FIG. 27C shows representative IF staining for HMGB1 in DMSO-treated, AA5-prevention- and AA5-resolution-treated iFA model revealing the absence or significantly reduced cytoplasmic HMGB1 in the AA5-treated cultures; Scale bar, 50 μm. Bottom panels are higher magnification of insets; Scale bar, 25 μm.

FIG. 27D shows an overlaid histogram plot that depict the relative SSEA4 fluorescence intensity in the iFA model with (red) and without (blue) AA5 treatment. Unstained control is depicted in grey. The percentage of SSEA4+ cells was reduced in the iFA model on AA5-resolution treatment.

### Comparison of the iFA Model to Fibrotic Lung

FIG. 29A shows enrichment analysis of canonical pathways (1,188 MSigDB canonical pathways) in transcriptome data of RNA sequencing in the iFA model compared to the previously published IPF lung tissue data. The heatmap indicates a general agreement (hypergeometric P-value 5.87×10-40) between pathways that were enriched in healthy lung tissue (n=8) and day cultures (pre-iFA phenotype, n=3), bottom, as compared to the IPF lung tissue (n=7) and day 13 cultures displaying iFA phenotypes (n=3), top. In FIGS. 29B-E, the most enriched pathways in either healthy or IPF phenotype are indicated. The average rank of the normalized enrichment scores (NES) and NES values are indicated.

FIG. 29B shows a heatmap showing expression of genes (red/blue are up/down-regulated), encoding for pathways in core matrisome. FIG. 29C shows a heatmap showing expression of genes (red/blue are up/down-regulated), encoding for pathways in the indicated factors. FIG. 29D shows a heatmap showing expression of genes (red/blue are up/down-regulated), encoding for pathways in integrin 1. FIG. 29E shows a heatmap showing expression of genes (red/blue are up/down-regulated), encoding for pathways in cytokine-cytokine receptor interaction. These heatmaps compare the iFA model and previously published IPF lung tissue. On the right of each heatmap, genes at the extremes of the lists are indicated. At the bottom of each panel, a plot showing running enrichment score (ES) across the ranked list in each pathway is indicated, where the score at the peak of the plot is the final enrichment of the given gene set.

### AA5 Suppresses Bleomycin-Induced Pulmonary Fibrosis in Aged Mice

FIG. 30A shows schema illustrating the experiment to induce and treat IPF in mice. Naive mice (n=4) without bleomycin were also monitored in the same manner throughout treatment period. FIG. 30B shows hydroxyproline content in lung tissue collected on day 21 post bleomycin injury with and without AA5, showing a significant decrease in collagen content post AA5 treatment (n=8). Data represent min-max and median of collagen content relative to total protein content. ***P<0.001 **P<0.01 using one-way ANOVA and Sidak's multiple comparison test. FIGS. 30C and 30D show a representative H&E section of bleomycin treated lungs with the borders of the lung and fibrotic areas marked using the spline contour tool (FIG. 30C). Percent fibrotic area was calculated for each lobe and plotted as shown in FIG. 30D. FIG. 30E shows H&E stained sections of bleomycin-treated lungs with and without AA5 collected at day 21. The leftmost panel shows sections of naïve mice. Scale bar, 200 μm. FIG. 30F shows representative IF images of naïve, vehicle and AA5 treated mice lungs following bleomycin injury stained for the fibrosis marker α-SMA, and alveolar type II marker proSPC. Scale bars, 100 μm.

### Immunofluorescence

Cells grown on coverslips were fixed in 4% paraformaldehyde (PFA) for 10 min at room temperature and washed three times with TBS buffer supplemented with 0.1% Tween 20 (TBST). Lung samples were fixed overnight in 4% PFA at room temperature, washed 3 times in PBS, dehydrated and then infiltrated and embedded with wax. Five-micron sections were collected on glass slides, deparaffinized and rehydrated. Heat-induced epitope retrieval was performed using 1 mM EDTA in a microwave. Prior to staining, the samples were permeabilized with 0.25% Triton-X 100 in TBS for 5 min, washed twice with TBST and blocked with protein block (Dako) for 30 min. The samples were then incubated with the primary antibodies listed in Table 3 for 1 hr at room temperature, washed thrice with TBST and incubated with secondary antibodies listed in Table 3 for 45 min at room temperature in the dark. The coverslips or slides were washed thrice with TBST, once with TBS, mounted using Vectashield containing DAPI. Images were captured on a LSM 780 confocal microscope (Zeiss) using ZEN 2011 software.

TABLE 3

| Antibody | Raised in | Dilution | Application | Catalog Number | Clone | Company |
|---|---|---|---|---|---|---|
| Fibroblast Surface | Mouse | 1:000 | IF | ab11333 | 1B10 | Abcam, Cambridge, MA |
| Protein-1 (FSP1) | | | | | | |
| Vimentin (VIM) | Rabbit | 1:200 | IF | bs-23063R | | Bioss, Wobum, MA |
| α-SMA | Mouse | 1:200, 1:1000 | IF, WB | A5228 | 1A4 | Sigma, St. Louis, MO |
| Collagen I | Rabbit | 1:200, 1:5000 | IF, WB | AF6220 | | R and D systems, MN |
| SOX2 | Rabbit | 1:200 | IF | 3579 | D6O8 | Cell Signaling |
| | | | | | | Technology. MA |
| Keratin (K) 5 | Rabbit | 1:500 | IF | AF109 | | Covance |
| NF-kB p65 | Rabbit | 1:100 | IF | bs-0465R | | Bioss, Wobum, MA |
| HMGB1 | Mouse | 1:100 | IF | WH0003146M8 | 2F6 | Sigma, St. Louis, MO |
| ACTIN | Rabbit | 1:1000 | WB | sc-7210 | H-196 | Santa Cruz Biotech, CA |
| PCNA | Rabbit | 1:1000 | IF | ab92552 | EPR3821 | Abcam, Cambridge, MA |
| CD163L1 | Mouse | 1:500 | IHC | ab126756 | EPR6539 | Abcam, Cambridge, MA |
| NPTX1 | Mouse | 1:100 | IF | 7707-NP-050 | TU-20 | Millipore |
| CD14-405 | Mouse | 5 ul/test | FACS | FAB3832V | 134620 | R and D systems, MN |
| CD68-PE | Mouse | 5 ul/test | FACS | 2-0681-82 | FA-11 | eBiosciences |
| CD32-FITC | Mouse | 5 ul/test | FACS | 11-0329-42 | 6C4 | eBiosciences |
| CD11b-APC | Rat | 5 ul/test | FACS | FAB1124A | M1/70 | R and D systems, MN |
| CD44-405 | Rat | 1:100 | FACS | FAB6127V | IM7.8.1R | R and D systems, MN |
| CD326-PE | Mouse | 1:100 | FACS | 324205 | 9C4 | Biolegend, San Diego |
| CD45-AlexaFluor-488 | Mouse | 1:100 | FACS | FAB1430G | 2D1 | R and D systems, MN |
| SSEA4-APC | Mouse | 1:200 | IF, FACS | FAB1435A | MC-813-70 | R and D systems, MN |
| CD105-Alexa 488 | Rat | 1:100 | FACS | 120405 | MJ7/18 | San Diego, CA |
| Anti-rabbit 488 | Donkey | 1:200 | IF | A-21206 | | Thermofisher, scientific |
| Anti-rabbit 594 | Donkey | 1:200 | IF | A-21207 | | Thermofisher, scientific |
| Anti-mouse 488 | Donkey | 1:200 | IF | A-21202 | | Thermofisher, scientific |
| Anti-mouse 594 | Donkey | 1:200 | IF | A-21203 | | Thermofisher, scientific |

### Detection of Cellular Senescence

iFA cultures were fixed in 1% formaldehyde/0.2% glutaraldehyde in PBS for 15 minutes at RT, rinsed in PBS and processed for β-galactosidase staining using, the senescence detection kit (BioVision) according to the manufacturer's instruction. The cultures were counter-stained with eosin and imaged.

### Transmission Electron Microscopy (TEM)

Cells in the iFA model were fixed with 2.5% glutaraldehyde in 0.1 M sodium cacodylate buffer, pH 7.4 for 30 min at RT, followed by overnight incubation at 4° C. Cells were then treated with 0.5% of tannic acid for an hr at RT, followed by wash with PBS buffer (5 times) and post fixed in a solution of 1% 0504 in PBS, pH 7.2-7.4. The samples were then washed with Na acetate buffer, pH 5.5 (4 times), block-stained in 0.5% uranyl acetate for 12 hr at 4° C. The samples were dehydrated in graded ethanol 10 minutes each, passed through propylene oxide, and infiltrated in mixtures of Epon 812 and propylene oxide 1:1 and then 2:1 for 2 hrs each and then infiltrated in pure Epon 812 overnight. Upon embedding and curing, sections of 60 nm thickness were cut on an ultramicrotome (RMC MTX). The sections were deposited carefully on single-hole grids coated with Formvar and carbon and double-stained in aqueous solutions of 8% uranyl acetate for 25 min at 60° C. and lead citrate for 3 minutes at RT. Thin sections subsequently were examined with a 100CX JEOL electron microscope.

### Immunoblot Analyses

Cells were lysed in RIPA lysis buffer with added protease inhibitor cocktail (Roche, USA). Protein concentrations were estimated using qubit fluorometer. Samples were prepared by adding an equal volume of 2×SDS sample buffer to the samples and denatured by boiling for 5 minutes. Samples were applied and separated through Mini-PROTEAN® TGX Stain-Free™ Precast Gels (Biorad) and transferred to an Immobilon PVDF membrane (Millipore, USA). The membranes were blocked with Tris-buffered saline with 0.05% Tween 20 and 5% skimmed milk for 30 minutes and then treated with primary antibodies (listed in Table 3). The preparative membranes were then incubated with appropriate secondary antibodies conjugated to horseradish peroxidase (Invitrogen). The immune-complexes were visualized with the ECL kit (GE-Healthcare, USA). Bands were quantified using Image Lab software/Gel Doc XR+ system, and values were normalized to either total protein lanes or actin levels.

### RNA Preparation and Expression Analysis

In-vitro cultures of primary fibroblasts and cells in the iFA model were washed once with PBS and total RNA from the samples were extracted using the RNeasy Mini Kit (Qiagen) according to the manufacturer's instructions. For the lung slice cultures (LSC), tissues were snap-frozen after treatment and stored at −80° C. until RNA isolation. Lung slices were homogenized using a handheld homogenizer and passing the homogenate through a Qiashredder (Qiagen). Total RNA from the LSC and cells from the disease model were extracted using the RNeasy Mini Kit (Qiagen) according to the manufacturer's instructions. The RNA concentrations were measured on a NanoDrop ND-1000 spectrophotometer. Single-stranded cDNA was synthesized from 200 ng of total RNA using Superscript IV and random hexamer primers (Invitrogen) in a volume of 20 μl. cDNA was then used for qRT-PCR analysis. PCR reactions were performed using Taqman Gene Expression Assay mix (Applied Biosystems) according to the manufactures instructions. Taqman probes are listed in Table 2. qRT-PCR reactions were performed using the StepOnePlus (Applied Biosystems). Relative gene expression was calculated using the 2^{-ΔΔCt} method, with 18S Cat. #4331182 (Invitrogen) as housekeeping gene. For the RT²qPCR arrays, cDNA from DMSO and AA5 treated iFA and was added to the RT² qPCR iTaq Universal SYBR green Master Mix (Biorad). 20 μl of the experimental cocktail was added to each well of the Fibrosis PCR (Qiagen). Real-Time PCR was performed on the StepOnePlus qPCR system (Applied Biosystems) using SYBR green detection according to the manufacturer's recommendations. All data from the PCR was collected and analyzed by SA Bioscience's PCR Array Data Analysis Web Portal.

### Fluorescence-Activated Cell Sorting (FACS)

For determination of various cell populations in the iFA model, the cells were dissociated using Accumax (Stem Cell Technologies) for 5 minutes, pelleted and resuspended in FACS buffer (3% Fetal Bovine Serum/PBS). 1×10⁶ cells were incubated with a the appropriate conjugated antibody listed in Table 3 for 20 minutes at 4° C. under shaking conditions. The cells were washed with the FACS buffer and acquired using a flow cytometer (BD LSRII) and analysed using FACS Diva and FlowJo softwares.

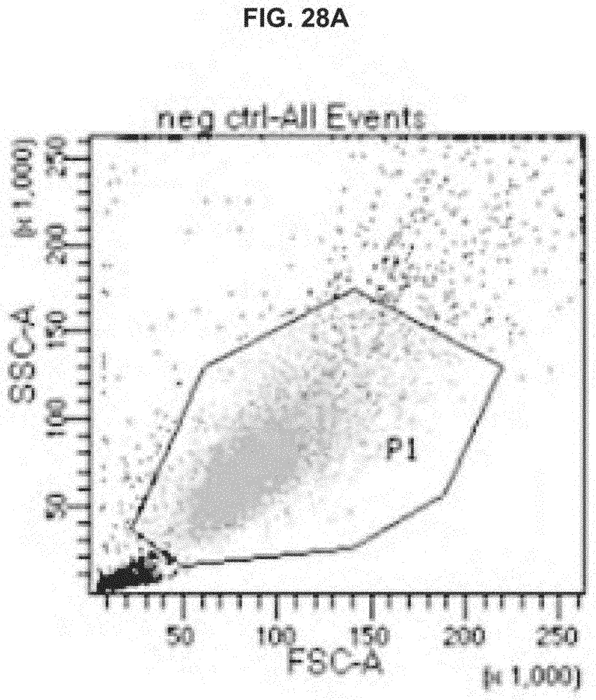

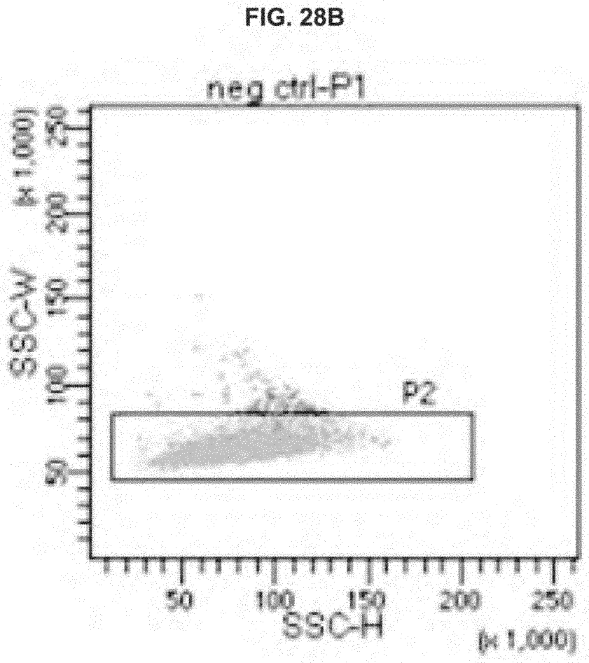

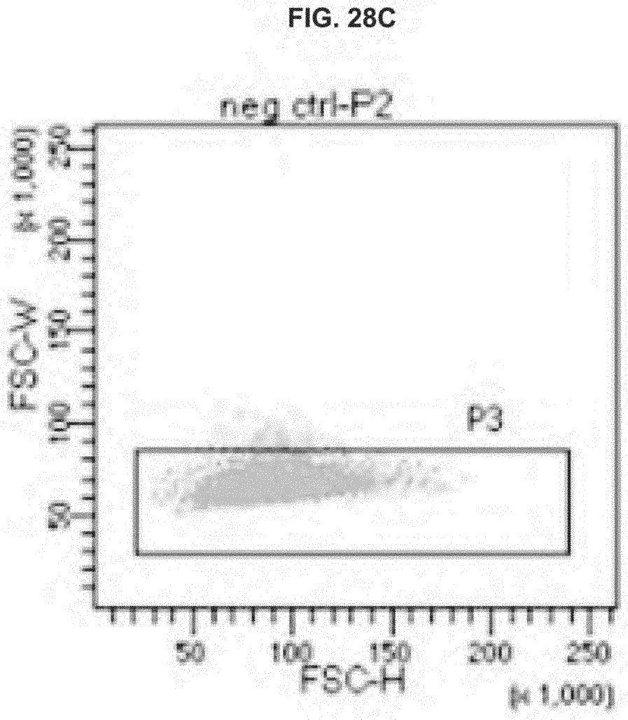

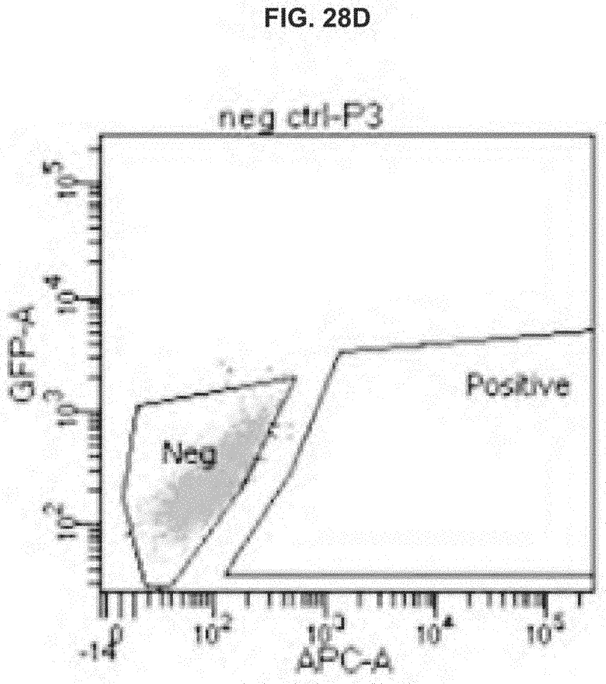

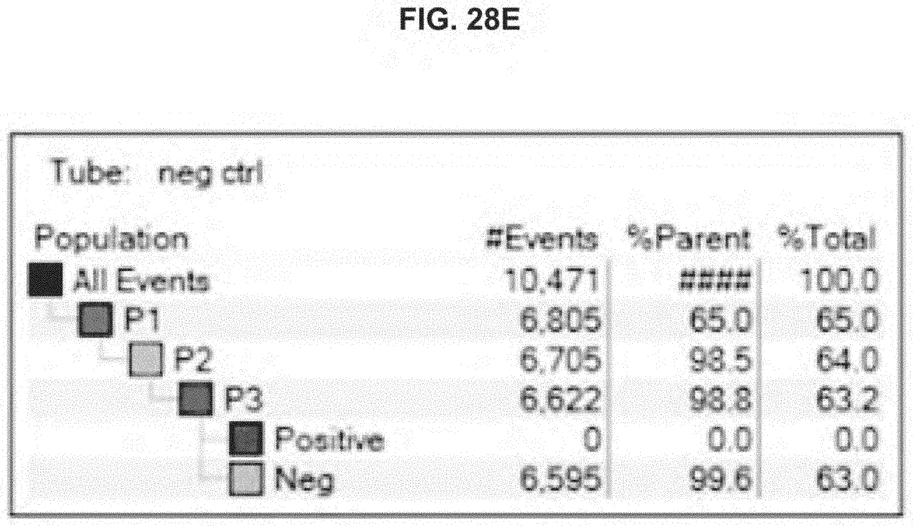

For dissociation of human lung slice cultures, 48 hours after DMSO or AA5 treatment, the samples were dissociated using the Multi Tissue Dissociation Kit (Milteny) (2.35 mL of DMEM, 100 μL of Enzyme D, 50 μL of Enzyme R, and 12.5 μL of Enzyme A) using gentleMACS Octo Dissociator at 37° C. for 40 minutes. The cell suspension was passed though 40 micron filter and pelleted. Erythrocytes were lysed using the Red Blood Lysis Solution (Milteny) for 2 minutes, pelleted and re-suspended in FACS buffer. Total cells were stained using the antibodies listed in Table 3 for 30 minutes followed by washing. The cells were resuspended in FACS buffer and analyzed by Flow Cytometry using the FACS Diva (BD Biosciences) and FlowJo softwares. Sample of the gating strategy is shown in FIGS. 28A-E.

FIG. 28 shows the gating strategy. Unstained samples were gated using forward and side scatter (FSC-A and SSC-A) (FIG. 28A) followed by SSC-W/SSC-H (FIG. 28B) and FSC-W/FSC-H (FIG. 28C) gating to select single cells. FIG. 28D shows negative cells were then gated using the unstained controls. FIG. 28E shows positive gating was then drawn using single stained controls.

### Quantification of Hydroxyproline Content in Conjunctival and LSCs

Conjunctiva or LSCs were boiled in 50 μl or 400 μl of 6 M HCl, respectively at 100° C. overnight. Hydroxyproline levels were measured in the acid hydrolysis method using a kit from Biovision Inc. (Milpitas, Calif.) using the manufacturer's instructions. The collagen content was estimated by either normalizing to total protein content (Conjunctiva) (Cedarlane) or by wet weight (LSC)

### Statistical Methods

Statistical methods for relevant figures are outlined above. Statistical analysis was performed using GraphPad Prism software Ver.7 (GraphPad, San Diego, Calif., USA) by one-way or two-way analysis of variance (ANOVA), with Tukey, Sidak or Dunnett tests for post-hoc analysis. Sample groups of n=5 or more, where each replicate (“n”) represents entirely separate iFA cultures from different patient lines (biological replicates) or lung slice cultures from multiple patients sliced from a different areas of the lung. Different areas were used to increase then value since the extent of fibrosis is heterogeneous even within a single patient sample. Biologically relevant differences between treatment groups would have a large effect size due to heterogeneity expected in patient samples. Thus, the sample size threshold was set to at least 5 replicates to have a large enough to perform analysis of variance. The p value threshold to determine significance was set at p=0.05. Data for quantitative experiments was represented as the mean with error bars representing standard error of the mean.

### REFERENCES

Bindea, G., Mlecnik, B., Hackl, H., Charoentong, P., Tosolini, M., Kirilovsky, A., Fridman, W. H., Pages, F., Trajanoski, Z., and Galon, J. (2009). ClueGO: a Cytoscape plug-in to decipher functionally grouped gene ontology and pathway annotation networks. Bioinformatics 25, 1091-1093.
Ekins, S., and Bunin, B. A. (2013). The Collaborative Drug Discovery (CDD) database. Methods in molecular biology 993, 139-154.
Inglese, J., Auld, D. S., Jadhav, A., Johnson, R. L., Simeonov, A., Yasgar, A., Zheng, W., and Austin, C. P. (2006). Quantitative high-throughput screening: a titration-based approach that efficiently identifies biological activities in large chemical libraries. Proc Natl Acad Sci USA 103,11473-11478.
Kariv, I. I., Stevens, M. E., Behrens, D. L., and Oldenburg, K. R. (1999). High Throughput Quantitation of cAMP Production Mediated by Activation of Seven Transmembrane Domain Receptors. J Biomol Screen 4, 27-32
Karumbayaram, S., Lee, P., Azghadi, S. F., Cooper, A. R., Patterson, M., Kohn, D. B., Pyle, A., Clark, A., Byrne, J., Zack, J. A., et al. (2012). From skin biopsy to neurons through a pluripotent intermediate under Good Manufacturing Practice protocols. Stem cells translational medicine 1, 36-43.
Koyama, N., Miura, M., Nakao, K., Kondo, E., Fujii, T., Taura, D., Kanamoto, N., Sone, M., Yasoda, A., Arai, H., et al. (2013). Human induced pluripotent stem cells differentiated into chondrogenic lineage via generation of mesenchymal progenitor cells. Stem cells and development 22, 102-113.
Mazzieri, R., Munger, J. S., and Rifkin, D. B. (2000). Measurement of active TGF-beta generated by cultured cells. Methods in molecular biology 142, 13-27.
Plaisier, S. B., Taschereau, R., Wong, J. A., and Graeber, T. G. (2010). Rank-rank hypergeometric overlap: identification of statistically significant overlap between gene-expression signatures. Nucleic Acids Res 38, e169.
Wilkinson, D. C., Alva-Ornelas, J. A., Sucre, J. M., Vijayaraj, P., Durra, A., Richardson, W., Jonas, S. J., Paul, M. K., Karumbayaram, S., Dunn, B., et al. (2016). Development of a Three-Dimensional Bioengineering Technology to Generate Lung Tissue for Personalized Disease Modeling. Stem cells translational medicine.
Wilkinson, D. C., Alva-Ornelas, J. A., Sucre, J. M., Vijayaraj, P., Durra, A., Richardson, W., Jonas, S. J., Paul, M. K., Karumbayaram, S., Dunn, B., et al. (2017). Development of a Three-Dimensional Bioengineering Technology to Generate Lung Tissue for Personalized Disease Modeling. Stem Cells Transl Med 6, 622-633.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing a fibrotic disease or disorder in a subject in need thereof, comprising administering to the subject an agent selected from 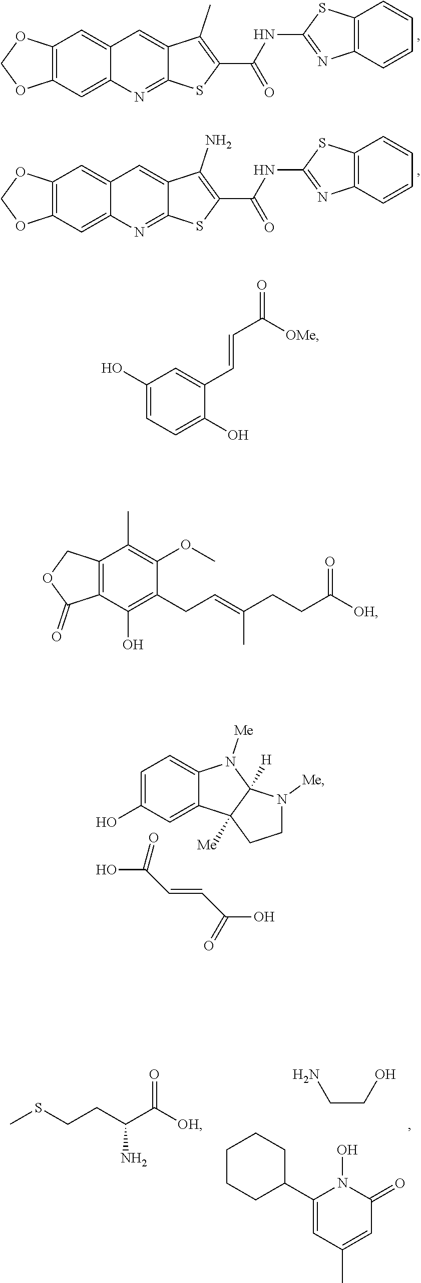 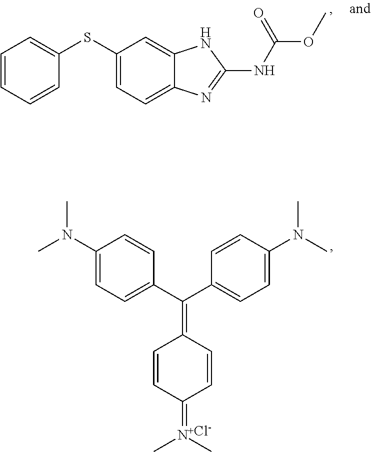
or a pharmaceutically acceptable salt thereof.

2. The method of claim 1, wherein the fibrotic disease or disorder is selected from liver disease, kidney disease, idiopathic pulmonary fibrosis (IPF), heart failure, scleroderma, rheumatoid arthritis, Crohn's disease, ulcerative colitis, myelofibrosis, systemic lupus erythematosus, tumor invasion and metastasis, and chronic graft rejection.

3. The method of claim 1, wherein the fibrotic disease or disorder is selected from systemic or local scleroderma, keloids, hypertrophic scars, atherosclerosis, restenosis, pulmonary inflammation and fibrosis, idiopathic pulmonary fibrosis, liver cirrhosis, fibrosis as a result of SARS-CoV-2 or chronic hepatitis B or C infection, kidney disease, heart disease resulting from scar tissue, macular degeneration, and retinal and vitreal retinopathy.

4. The method of claim 1, wherein the method inhibits excessive fibrosis formation occurring in the liver, kidney, lung, heart or pericardium, eye, skin, mouth, pancreas, gastrointestinal tract, brain, breast, bone marrow, bone, genitourinary, a tumor, or a wound.

5. A method of inhibiting fibrosis in a subject in need thereof, comprising administering to the subject an agent selected from: 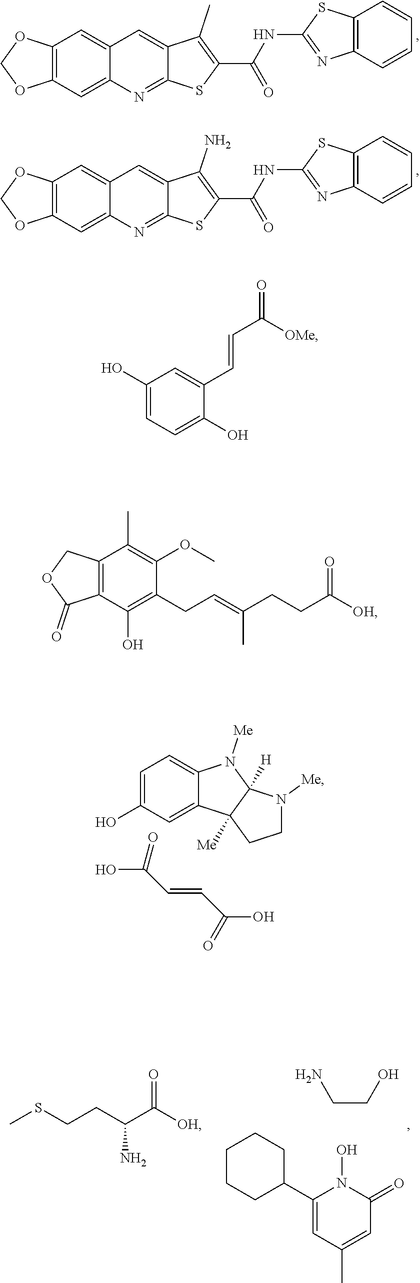 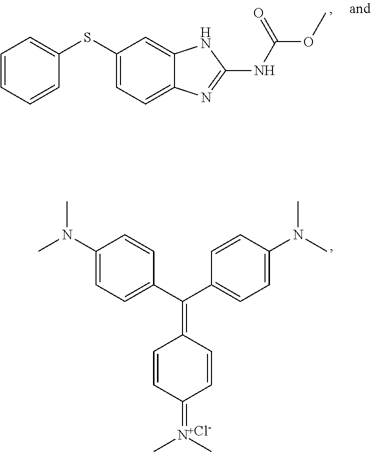
or a pharmaceutically acceptable salt thereof.

6. The method of claim 5, wherein the fibrosis comprises progressive fibrosis.

7. The method of claim 5, wherein the fibrosis is fibroproliferative.

8. The method of claim 5, wherein the fibrosis is viral-infection-induced fibrosis, e.g., incident to COVID-19.

9. The method of claim 1, wherein the agent is

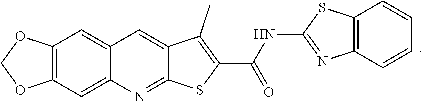 [SMILES: Cc1c(C(=O)Nc2nc3ccccc3s2)sc2nc3cc4c(cc3cc12)OCO4]

10. The method of claim 1, wherein the agent is

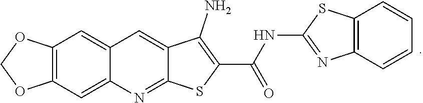 [SMILES: Nc1c(C(=O)Nc2nc3ccccc3s2)sc2nc3cc4c(cc3cc12)OCO4]

11. The method of claim 1, wherein the agent comprises

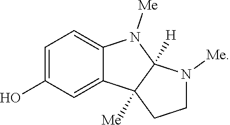 [SMILES: CN1CC[C@@]2(C)c3cc(O)ccc3N(C)[C@@H]12]

12. The method of claim 1, wherein the agent comprises

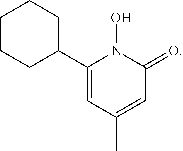

13. The method of claim 1, wherein the agent is present in a pharmaceutical composition comprising a pharmaceutically acceptable carrier.

14. The method of claim 1, wherein the agent is

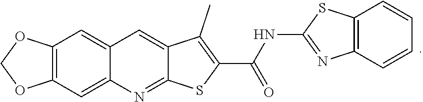 [SMILES: Cc1c(C(=O)Nc2nc3ccccc3s2)sc2nc3cc4c(cc3cc12)OCO4]

15. The method of claim 1, wherein the agent is

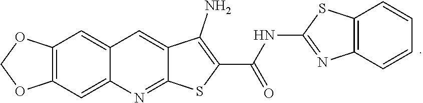 [SMILES: Nc1c(C(=O)Nc2nc3ccccc3s2)sc2nc3cc4c(cc3cc12)OCO4]

16. The method of claim 1, wherein the agent comprises

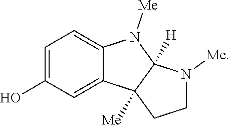 [SMILES: CN1CC[C@@]2(C)c3cc(O)ccc3N(C)[C@@H]12]

17. The method of claim 1, wherein the agent comprises

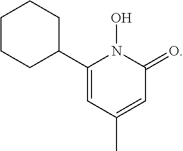

18. The method of claim 1, wherein the agent is present in a pharmaceutical composition comprising a pharmaceutically acceptable carrier.
